(19) United States
(12) Patent Application Publication
(10) Pub. No.: US 2026/0308686 A1
(43) Pub. Date: Oct. 8, 2026

(54) GENOMIC ALTERATIONS IN THE TUMOR AND CIRCULATION OF PANCREATIC CANCER PATIENTS

(71) Applicant: The Johns Hopkins University, Baltimore, MD (US)

(72) Inventors: Victor Velculescu, Dayton, MD (US); Mark Sausen, Baltimore, MD (US); Vilmos Adleff, Baltimore, MD (US); Jillian Phallen, Baltimore, MD (US)

(21) Appl. No.: 19/456,431

(22) Filed: Jan. 22, 2026

Related U.S. Application Data
(63) Continuation of application No. 18/394,161, filed on Dec. 22, 2023, now Pat. No. 12,559,802.
Application No. 18/394,161 is a continuation of application No. 18/135,534, filed on Apr. 17, 2023, now abandoned.
Application No. 18/135,534 is a continuation of application No. 17/080,201, filed on Oct. 26, 2020, now abandoned.
Application No. 17/080,201 is a division of application No. 15/552,076, filed on Feb. 18, 2016, now Pat. No. 10,815,522.
(60) Provisional application No. 62/118,604, filed on Feb. 20, 2015.

(51) Int. Cl. C12Q 1/6886; C12Q 1/6816; C12Q 1/6827

(52) U.S. Cl. CPC C12Q 1/6886; C12Q 1/6816; C12Q 1/6827; C12Q 2600/106; C12Q 2600/112; C12Q 2600/118; C12Q 2600/156

(57) ABSTRACT

Pancreatic adenocarcinoma has the worst overall mortality of any solid tumor, with only 7% of patients surviving after 5 years. To evaluate the clinical implications of genomic alterations in this low cellularity tumor type, we deeply sequenced the genomes of 101 enriched pancreatic adenocarcinomas from patients who underwent potentially curative resections and used non-invasive approaches to examine tumor specific mutations in the circulation of these patients. These analyses revealed somatic mutations in chromatin regulating genes including MLL and ARID1A in 20% of patients that were associated with improved survival. Liquid biopsy analyses of cell free plasma DNA revealed that 43% of patients with localized disease had detectable circulating tumor DNA (ctDNA) in their blood at the time of diagnosis. Detection of ctDNA after resection predicted clinical relapse and poor outcome, and disease recurrence by ctDNA was detected 6.5 months earlier than with standard CT imaging.

### CROSS-REFERENCE TO RELATED APPLICATIONS

This application is a continuation of and claims priority to U.S. application Ser. No. 18/135,534, filed Apr. 17, 2023, which is a continuation of and claims priority to U.S. application Ser. No. 17/080,201, filed Oct. 26, 2020, which is a divisional of and claims priority to U.S. application Ser. No. 15/552,076, filed Aug. 18, 2017, now U.S. Pat. No. 10,815,522, which is a National Stage application under 35 U.S.C. § 371 of International Application No. PCT/US2016/018450, filed Feb. 18, 2016, which claims the benefit of U.S. Provisional Application No. 62/118,604, filed Feb. 20, 2015. The disclosures of the prior applications are hereby incorporated by reference in their entirety.

### FEDERALLY SPONSORED RESEARCH OR DEVELOPMENT

This invention was made with Government support under grant CA121113, awarded by the National Institutes of Health. The Government has certain rights in the invention.

### TECHNICAL FIELD OF THE INVENTION

This invention is related to the area of cancer. In particular, it relates to pancreatic cancer.

### BACKGROUND OF THE INVENTION

Worldwide, over 250,000 patients develop pancreatic ductal adenocarcinoma every year and the vast majority die of their disease1. Several genetic alterations have been identified in pancreatic cancers, including those in the CDKN2A, SMAD4 and TP53 tumor suppressor genes, and in the KRAS oncogene2′3. Although the discoveries of these genes and their pathways have provided important insights into the natural history of pancreatic cancer and have spurred efforts to develop improved diagnostic and therapeutic agents, few genetic alterations discovered to date in pancreatic cancer have been used to directly affect clinical care4,5.

There is a continuing need in the art to develop tools for detecting disease, assessing disease, and effectively treating disease, particularly highly lethal disease such as pancreatic cancers.

### SUMMARY OF THE INVENTION

According to one aspect of the invention a method is provided for characterizing a pancreatic adenocarcinoma. A sample of nucleic acids from a pancreatic adenocarcinoma of a human is tested to identify a somatic mutation in a chromatin regulating gene selected from the group consisting of MLL, MLL2, MLL3, and ARID1A.

Another aspect of the invention is a method for testing a human. Plasma of a human collected at the time of diagnosis of a pancreatic adenocarcinoma is tested for the presence of cell-free tumor DNA. The cell-free tumor DNA contains a somatic mutation present in the pancreatic adenocarcinoma but not present in tissues that are not pancreatic adenocarcinoma.

An additional aspect of the invention is a method for testing a human. Plasma of a human collected after surgical resection of a pancreatic adenocarcinoma is tested for the presence of cell-free tumor DNA. The cell-free tumor DNA contains a somatic mutation present in the pancreatic adenocarcinoma but not present in tissues that are not pancreatic adenocarcinoma.

One aspect of the invention is a probe or primer specific for a mutant chromatin regulating MLL (chromosome 11), MLL2 (chromosome 12), MLL 3 (chromosome 7) or ARID1A gene, said probe or primer comprising a mutation selected from the group consisting of:

ARID1A mutations:

| | | | |
|---|---|---|---|
| chr1_26928798- | 403Q > X | Substitution | Nonsense; |
| 26928798_C_T | | | |
| chr1_26979465- | NA | Insertion | Frameshift; |
| 26979465_C | | | |
| chr1_26978312- | 1779E > G | Substitution | Nonsynonymous |
| 26978312_A_G | | | coding; |
| chr1_26978589- | 1871H > Q | Substitution | Nonsynonymous |
| 26978589_C_G | | | coding; |
| chr1_26895595- | 38E > EA | Insertion | In-frame |
| 26895595_GGC | | | insertion; |
| chr1_26960135- | 708Q > X | Substitution | Nonsense; |
| 26960135_C_T | | | |
| chr1_26973560- | 1419A > T | Substitution | Nonsynonymous |
| 26973560_G_A | | | coding; |
| chr1_26974706- | 1682A > E | Substitution | Nonsynonymous |
| 26974706_C_A | | | coding; |
| chr1_26972534- | 1276R > X | Substitution | Nonsense; |
| 26972534_C_T | | | |
| chr1_26978518- | NA | Deletion | Frameshift; |
| 26978518_G_ | | | |
| chr1_26979254- | NA | Insertion | Frameshift; |
| 26979254_TT | | | |

and MLL (chromosome 11), MLL2 (chromosome 12), or MLL 3 (chromosome 7) mutations:

| | | | |
|---|---|---|---|
| chr7_151490485- | 3704V > L | Substitution | Nonsynonymous |
| 151490485_C_G | | | coding; |
| chr11_117854039- | 1161C > S | Substitution | Nonsynonymous |
| 117854039_G_C | | | coding; |
| chr11_117847769- | 229P > T | Substitution | Nonsynonymous |
| 117847769_C_A | | | coding; |
| chr12_47718147- | 3087R > W | Substitution | Nonsynonymous |
| 47718147_G_A | | | coding; |
| chr7_151515732- | NA | Substitution | Splice |
| 151515732_C_T | | | site donor; |
| chr12_47729834- | NA | Insertion | Frameshift; |
| 47729834_A | | | |
| chr7_151576224- | 743P > L | Substitution | Nonsynonymous |
| 151576224_G_A | | | coding; |
| chr7_151476195- | 4584R > W | Substitution | Nonsynonymous |
| 151476195_G_A | | | coding; |
| chr7_151686622- | NA | Insertion | Frameshift; |
| 151686622_TC | | | |
| chr12_47711653- | NA | Insertion | Frameshift; |
| 47711653_T | | | |
| chr12_47731362- | NA | Deletion | Frameshift; |
| 47731362_G_ | | | |
| chr7_151601824- | NA | Insertion | Frameshift; |
| 151601824_T | | | |
| chr12_47722327- | 1974T > M | Substitution | Nonsynonymous |
| 47722327_G_A | | | coding; and |
| chr7_151510210- | 1890R > X | Substitution | Nonsense. |
| 151510210_G_A | | | |

These and other embodiments which will be apparent to those of skill in the art upon reading the specification provide the art with

### BRIEF DESCRIPTION OF THE DRAWINGS

FIG. 1. Schematic of next-generation sequencing and circulating tumor DNA analyses.

FIG. 2A-2C. Recurrent genetic alterations in pancreatic cancer and their effect on disease outcome. FIG. 2A: Representation of the mutations identified in chromatin modifying and other recurrently mutated genes. Each patient sample is indicated as a grey box with mutations indicated in green or black. FIG. 2B: Analyses of overall survival revealed that patients wild type for MILL gene alterations (n=87) had a significantly lower median survival compared with those with mutated MLL genes (n=14; median survival 15.3 months vs not reached respectively, p=0.0063, log-rank test). FIG. 2C: Similar analyses revealed significantly improved survival in patients with mutations in either MLL or ARIDIA genes (n=20) compared with those that were wild-type for any of these genes (n=81).

FIG. 3A-3B. Detection of residual disease using CT imaging and ctDNA analyses. FIG. 3A: Patients with detectable circulating tumor DNA (ctDNA) after surgical resection were more likely to relapse and die from disease compared to those with undetectable ctDNA. The median time to recurrence was 9.9 months for individuals with detectable ctDNA and was not reached for those without detectable ctDNA. FIG. 3B: Comparison between the time to detection of recurrence using ctDNA and standard-of-care CT imaging revealed that the median time to recurrence was 71 days for individuals with detectable ctDNA and 289 days for those patients with positive imaging results (p=0.0004, paired t-test).

FIG. 4 (Table 1) Clinical Actionability of Genetic Alterations in Pancreatic Cancer

FIG. 5. (Supplementary FIG. 1.) Schematic of Mutations in MLL and ARID1A Proteins. Domains indicated in the schematic represent PHD, plant homeo domain finger, HMG, high mobility group box, FYRN, FY-rich N-terminal domain, FY-rich C-terminal domain, SET, Su (var)3-9 Enhancer-of-zeste Trithorax methyltransferase domain, BROM, bromodomain, ARID, ARID/BRIGHT DNA binding domain, DUF3518, and Domain of unknown function (DUF3518). Mutations were assessed for conservation by phyloP across 44 vetebrate species and those highlighted in green were predicted to be conserved. Black arrows indicate missense alterations and red arrows indicate truncating alterations.

FIG. 6A-6B. (Supplementary FIG. 2.) Kaplan Meier Progression-Free Survival Analysis of MLL and Chromatin Regulator Mutations. FIG. 6A: The hazard ratio for tumor recurrence among individuals with mutant MLL, MLL2, or MLL3 genes compared to those with wild-type MLL, MLL2, or MLL3 genes (WT) was 0.54 (95% CI=0.30-0.98). The median time to recurrence was 10.2 months for individuals wild-type for MLL gene alterations and 24.9 months for those with mutated MLL genes. FIG. 6B: The hazard ratio for tumor recurrence among individuals with mutant chromatin regulator genes compared to those with wild-type (WT) chromatin regulator genes was 0.47 (95% CI=0.27-0.82). The median time to recurrence was 8.2 months for individuals wild-type for chromatin regulator gene alterations and 24.9 months for those with mutated chromatin regulator genes.

FIG. 7. (Supplementary FIG. 3.) Prediction of Recurrence using ctDNA Detected at Baseline. The hazard ratio for tumor recurrence among individuals with detectable circulating tumor DNA (ctDNA) compared to those without detectable ctDNA was 2.39 (95% CI=1.18-4.81). The median time to recurrence was 15.2 months for individuals without detectable ctDNA and 7.9 months for those with detectable ctDNA.

FIG. 8 (Supplemental Table 1)

FIG. 9 (Supplemental Table 4 (ARID1A, MLL, MLL2, AND MHLL3)

FIG. 10 (Supplemental Table 5)

FIG. 11 (Supplemental Table 7)

FIG. 12 (Supplemental Table 8)

### DETAILED DESCRIPTION OF THE INVENTION

Through integrated genomic analyses we have identified MLL and ARIDIA genes as markers of improved prognosis, and highlighted clinically actionable alterations in genes not typically evaluated during clinical care of pancreatic cancer patients. We have also shown that cell-free DNA in the circulation of pancreatic cancer patients may provide a marker of early detection of sub-clinical, residual or recurrent disease. These analyses suggest more intensive therapies for pancreatic cancer patients without MLL or ARIDIA alterations or with detectable cell-free DNA at the time of diagnosis or after surgical resection.

Testing for mutations in nucleic acids from plasma, other body fluids, or from tumor tissue can be performed by any means known in the art. Hybridization methods using probes may be used in any assay format desired, including but not limited to Southern or Northern blots, and microarrays. The probes may be double or single stranded. They may hybridize to the sense strand of the gene or the anti-sense strand. Mutations may also be detected using an amplification method, whether linear or exponential. The amplification may utilize one or more primers. The primers maybe contain a mutant nucleotide or be adjacent to it. A primer pair may surround a mutant nucleotide and the detection of the mutant may rely on hybridization to a probe after amplification. Other means for identifying mutations include nucleic acid sequencing techniques. Probes and primers may be labeled with any detectable label known in the art, including but not limited to radionuclides, fluorescent labels, chromophores, enzymes. Probes or primers may contain non-naturally occurring components, for example, to limit degradation. Artificial nucleic acids may include peptide nucleic acid, morpholino and locked nucleic acid, glycol nucleic acid, and threose nucleic acid (TNA). Each of these is distinguished from naturally occurring DNA or RNA by changes to the backbone of the molecule.

Nucleic acids from pancreatic adenocarcinomas may be obtained from biopsies or from surgical samples. Nucleic acids found in the plasma of a patient with a tumor are believed to be shed into the circulation or other body fluids, perhaps by cells which are undergoing apoptosis or necrosis. The presence of a somatic mutation that is identical to one found in the tumor indicates that the nucleic acid ultimately derived from the tumor.

Control nucleic acids for determining a somatic mutation may be taken from any normal tissue, including normal portions of the patient's pancreas, or other organs. If a mutation is a germline mutation, it will be found in all of a patient's tissues. In the case of circulating tumor DNA found in the plasma, this DNA typically forms a minority of the DNA found in the plasma.

Mutations that affect a coding sequence include substitutions, insertions, and deletions. These may cause nonsense, frameshifts, splice site donor, or non-synonymous coding mutations. Other than the latter type, these mutation types will typically lead to truncation of an encoded message or product.

Plasma, other body fluid, and/or tissue samples can be collected for testing at the time of diagnosis, before surgical resections, or after surgical resections. The findings of mutations or no mutations can inform subsequent treatment options. In some cases, surgery may be averted if a good prognosis is determined. In other cases more intensive therapies may be used to counter a bad prognostic finding. In other cases less intensive therapies may be used when a favorable prognostic indication is found. Therapies may be more or less intensive in kind, dosage, or duration.

The finding of mutations in the chromatin regulation genes is a positive prognostic indicator. Finding of tumor-specific, cell-free DNA is a negative prognostic indicator. Conversely, finding of no tumor-specific, cell-free DNA can be considered a positive prognostic indicator and finding of no chromatin regulation gene mutations may be considered a negative prognostic indicator. These factors affect both disease-free progression time as well as overall survival time. Prognoses can be delivered to a patient in a written or electronic form. They can be delivered from a clinical laboratory to a practicing physician.

Cell-free tumor DNA can be tested for mutations in any genes that are known to be associated with cancers. Often a single patient will have many somatic mutations in his tumor. Any of these can be detected and followed in the cell-free DNA of the plasma or other body fluid. Frequently mutated genes in pancreatic adenocarcinomas include KRAS, BRAF, and PIK3CA. Other frequently mutated genes may be monitored.

Plasma or other body fluids may be tested for the presence of cell-free DNA. Suitable body fluids include serum, blood, pancreatic juice, pus, stool, etc. Drainage of pancreatic lesions into the stomach or jejunum may lead to increased cell-free pancreatic DNA in the stool.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### Example 1—Materials and Methods

### Samples Obtained for Sequencing Analyses

Pancreatic ductal adenocarcinoma tumor specimens and matched germline specimens (from peripheral blood) from 101 patients were used for genomic analyses. Plasma samples were obtained at the time of diagnosis from 44 of these patients as well as 7 additional patients. Informed consent for research use was obtained from all patients at the enrolling institution prior to tissue banking and study approval was obtained. Primary tumor samples for genomic analyses were selected from patients with resectable stage II disease, verified to have >10% viable tumor cell content by histopathological assessment, and demonstrated to be wild-type for the DAXX/ATRXloci, which have been shown to be associated with improved outcome in patients with pancreatic neuroendocrine tumorsPEVuZE5vdGU+PENpdGU+PEF1dGhvcj5KaWFvPC9BdXRob3I+PFllYXI+Mj AxMTwvWWVhcj48UmVj TnVtPjE2PC9SZWNOdW0+PERpc3BsYXIUZXhOPjxzdHlsZSBmYWNlPSJzdXBlcnNj cmlwdCI+MjA8 L3N0eWxlPjwvRGlzcGxheVRleHQ+PHJlY29yZD48cmVjLW51bWJlcj4xNjwvcmVjL W51bWJlcj48 Zm9yZWlnbi1rZXlzPjxrZXkgYXBwPSJFTiIgZGItaWQ9IndkdmQyeDJ3NDJkOXgzZX h4YW54NWRw ZXJkZWRwdHRhMnhmeCI+MTY8L2tleT48L2ZvcmVpZ24ta2V5cz48cmVmLXR5cG UgbmFtZT0iSm91 cm5hbCBBcnRpY2xlIj4xNzwvcmVmLXR5cGU+PGNvbnRyaWJ1dG9ycz48YXV0aG9 ycz48YXV0aG9y PkppYW8sIFkuPC9hdXRob3I+PGF1dGhvcj5TaGksIEMuPC9hdXRob3I+PGF1dGhvcj 5FZG1sLCBC LiBILjwvYXV0aG9yPjxhdXRob3I+ZGUgV21sZGUsIFIuIEYuPC9hdXRob3I+PGF1dG hvcj5LbGlt c3RyYSwgRC4gUy48L2F1dGhvcj48YXV0aG9yPk1haXRyYSwgQS48L2F1dGhvcj48 YXV0aG9yP1Nj aHVsaWNrLCBSLiBELjwvYXV0aG9yPjxhdXRob3I+VGFuZywgTC4gSC48L2F1dGh vcj48YXV0aG9y P1dvbGZnYW5nLCBDLiBMLjwvYXV0aG9yPjxhdXRob3I+Q2hvdGksIEOuIEEuPC9h dXRob3I+PGF1 dGhvcj5WZWxjdWxlc2N1LCBWLiBFLjwvYXV0aG9yPjxhdXRob3I+RGlheiwgTC4g QS4sIEpyLjwv YXV0aG9yPjxhdXRob3I+Vm9nZWxzdGVpbiwgQi48L2F1dGhvcj48YXV0aG9yPktpb npsZXIsIEsu IFcuPC9hdXRob3I+PGF1dGhvcj5IcnViYW4sIFIuIEguPC9hdXRob3I+PGF1dGhvcj5Q YXBhZG9w b3Vsb3MsIE4uPC9hdXRob3I+PC9hdXRob3JzPjwvY29udHJpYnVOb3JzPjxhdXRoLW FkZHJ1c3M+THVkd2lnIENlbnRlciBmb3IgQ2FuY2VyIEdlbmV0aWNzIGFuZCBIb3dhcmQgSHVna GVzIE11ZGlj YWwgSW5zdG1OdXRpb25zLCBKb2hucyBIb3BraW5zIEtpbW11bCBDYW5jZXIgQ2V udGVyLCBCYWxO aW1vemUsIE1EIDIxMjMxLCBVUOEuPC9hdXRoLWFkZHJlc3M+PHRpdGxlcz48dGl ObGU+REFYWC9B VFJYLCBNRU4xLCBhbmQgbVRPUiBwYXRod2F5IGdlbmVzIGFyZ SBmcmVxdWV udGx5IGFsdGVyZWQg aW4gcGFuY3J1YXRpYyBuZXVyb2VuZG9jcmluZSBOdW1vcnM8L3RpdGxlPjxzZWN vbmRhcnktdGl0 bGU+U2NpZW5jZTwvc2Vjb25kYXJ5LXRpdGxlPjxhbHQtdGl0bGU+U2NpZW5jZTw vYWx0LXRpdGxl PjwvdGl0bGVzPjxwZXJpb2RpY2FsPjxmdWxsLXRpdGxlPlNjaWVuY2U8L2Z1bGwtd Gl0bGU+PGFi YnItMT5TY211bmN1PC9hYmJyLTE+PC9wZXJpb2RpY2FsPjxhbHQtcGVyaW9kaWNh bD48ZnVsbC10 aXRsZT5TY211bmN1PC9mdWxsLXRpdGxlPjxhYmJyLTE+U2NpZW5jZTwvYWJiciOx PjwvYWx0LXBI cmlvZGljYWw+PHBhZ2VzPjExOTktMjAzPC9wYWdlcz48dm9sdWllPjMzMTwvdm9 sdWllPjxudWli ZXI+NjAyMTwvbnVtYmVyPjxrZX13b3Jkcz48a2V5d29yZD5BZGFwdG9yIFByb3Rla W5zLCBTaWdu YWwgVHJhbnNkdWNpbmcvKmdlbmV0aWNzPC9rZX13b3JkPjxrZX13b3JkPkNhcmN pbm9tYSwgUGFu Y3J1YXRpYyBEdWN0YWwvZ2VuZXRpY3M8L2tleXdvcmQ+PGtleXdvcmQ+Q2hyb 21hdGluIEFzc2Vt Ymx5IGFuZCBEaXNhc3NlbWJseS9nZW51dGljczwva2V5d29yZD48a2V5d29yZD5ET kEgSGVsaWNh c2VzLypnZW5ldGljczwva2V5d29yZD48a2V5d29yZD5HZW5lcywgVHVtb3IgU3Vwc HJlc3Nvcjwv a2V5d29yZD48a2V5d29yZD5IdW1hbnM8L2tleXdvcmQ+PGtleXdvcmQ+Kk11dGFOa W9uPC9rZX13 b3JkPjxrZX13b3JkPk51dXJvZW5kb2NyaW51IFR1bW9ycy8qZ2VuZXRpY3MvbWVOY WJvbGlzbTwv a2V5d29yZD48a2V5d29yZD50dWNsZWFyIFByb3R1aW5zLypnZW5ldGljczwva2V5d 29yZD48a2V5 d29yZD5QVEVOIFBob3NwaG9oeWRyb2xhc2UvZ2VuZXRpY3M8L2tleXdvcmQ+PG tleXdvcmQ+UGFu Y3J1YXRpYyBOZW9wbGFzbXMvKmdlbmV0aWNzL21ldGFib2xpc208L2tleXdvcmQ+PGtleXdvcmQ+UGhvc3BoYXRpZH1saW5vc210b2wgMy1LaW5hc2VzL2dlbmV0aWNzPC9rZX13b3Jk PjxrZX13b3Jk PlByb2dub3Npczwva2V5d29yZD48a2V5d29yZD5Qcm90by1PbmNvZ2VuZSBQcm90 ZWlucy8qZ2Vu ZXRpY3M8L2tleXdvcmQ+PGtleXdvcmQ+U2VxdWVuY2UgQW5hbHlzaXMsIEROQ Twva2V5d29yZD48 a2V5d29yZD5TaWduYWwgVHJhbnNkdWNOaW9uL2dlbmV0aWNzPC9rZX13b3JkPjx rZX13b3JkP1RP UiBTZXJpbmUtVGhyZW9uaW51IEtpbmFzZXMvZ2VuZXRpY3MvKm1ldGFib2xpc20 8L2tleXdvcmQ+PGtleXdvcmQ+VHVtb3IgU3VwcHJlc3NvciBQcm90ZWlucy9nZW5ldGljczwva2V5d29 yZD48L2tl eXdvcmRzPjxkYXRlcz48eWVhcj4yMDExPC95ZWFyPjxwdWItZGF0ZXM+PGRhdG U+TWFyIDQ8L2Rh dGU+PC9wdWItZGF0ZXM+PC9kYXRlcz48aXNibj4xMDk1 LTkyMDMgKEVsZWNO cm9uaWMpJiN4RDsw MDM2LTgwNzUgKExpbmtpbmcpPC9pc2JuPjxhY2Nlc3Npb24tbnVtPjIxMjUyMzE1P C9hY2N1c3Np b24tbnVtPjxl cmxzPjxyZWxhdGVkLXVybHM+PHVybD5odHRwOi8vd3d3Lm5jYmku bmxtLm5paC5n b3YvcHVibWVkLzIxMjUyMzE1PC91cmw+PC9yZWxhdGVkLXVybHM+PC91cmxzP jxjdXN0b20yPjMx NDQOOTY8L2N1c3RvbTI+PGVsZWN0cm9uaWMtcmVzb3VyY2UtbnVtPjEwLjExMj Yvc2NpZW5jZS4x MjAwNjA5PC91bGVjdHJvbmljLXJlc291cmN1LW51bT48L3J1Y29yZD48LONpdGU+P C9FbmROb3R1 PgB=PEVuZE5vdGU+PENpdGU+PEF1dGhvcj5KaWFvPC9BdXRob3I+PFllYXI+MjAxMT wvWWVhcj48UmVj TnVtPjE2PC9SZWNOdW0+PERpc3BsYX1UZXhOPjxzdHlsZSBmYWN1PSJzdXBlcnNj cmlwdCI+MjA8 L3NOeWx1PjwvRGlzcGxheVRleHQ+PHJ1Y29yZD48cmVjLW51bWJlcj4xNjwvcmVjL W5lbWJlcj48 Zm9yZWlnbi1rZXlzPjxrZXkgYXBwPSJFTiIgZGItaWQ9IndkdmQyeDJ3NDJkOXgzZX h4YW54NWRw ZXJkZWRwdHRhMnhmeCI+MTY8L2tleT48L2ZvcmVpZ24ta2V5cz48cmVmLXR5cG UgbmFtZT0iSm91 cm5hbCBBcnRpY2xlIj4xNzwvcmVmLXR5cGU+PGNvbnRyaWJ1dG9ycz48YXV0aG9 ycz48YXV0aG9y PkppYW8sIFkuPC9hdXRob3I+PGF1dGhvcj5TaGksIEMuPC9hdXRob3I+PGF1dGhvcj 5FZG1sLCBC LiBILjwvYXV0aG9yPjxhdXRob3I+ZGUgV21sZGUsIFIuIEYuPC9hdXRob3I+PGF1dG hvcj5LbGlt c3RyYSwgRC4gUy48L2F1dGhvcj48YXV0aG9yPk1haXRyYSwgQ S48L2F1dGhvcj48 YXV0aG9yP1Nj aHVsaWNrLCBSLiBELjwvYXV0aG9yPjxhdXRob3I+VGFuZywgTC4gSC48L2F1dGh vcj48YXV0aG9y P1dvbGZnYW5nLCBDLiBMLjwvYXV0aG9yPjxhdXRob3I+Q2hvdGksIEOuIEEuPC9h dXRob3I+PGF1 dGhvcj5WZWxjdWxlc2N1LCBWLiBFLjwvYXV0aG9yPjxhdXRob3I+RGlheiwgTC4g QS4sIEpyLjwv YXV0aG9yPjxhdXRob3I+Vm9nZWxzdGVpbiwgQi48L2F1dGhvcj48YXV0aG9yPktpb npsZXIsIEsu IFcuPC9hdXRob3I+PGF1dGhvcj5IcnViYW4sIFIuIEguPC9hdXRob3I+PGF1dGhvcj5Q YXBhZG9w b3Vsb3MsIE4uPC9hdXRob3I+PC9hdXRob3JzPjwvY29udHJpYnVOb3JzPjxhdXRoLW FkZHJ1c3M+THVkd2lnIENlbnRlciBmb3IgQ2FuY2VyIEdlbmV0aWNzIGFuZCBIb3dhcmQgSHVna GVzIE11ZGlj YWwgSW5zdG10dXRpb25zLCBKb2hucyBIb3BraW5zIEtpbW11bCBDYW5jZXIgQ2V udGVyLCBCYWxO aW1vemUslE1EIDIxMjMxLCBVUOEuPC9hdXRoLWFkZHJlc3M+PHRpdGxlcz48dGl ObGU+REFYWC9B VFJYLCBNRU4xLCBhbmQgbVRPUiBwYXRod2F5IGdlbmVzIGFyZSBmcmVxdWV udGx5IGFsdGVyZWQg aW4gcGFuY3J1YXRpYyBuZXVyb2VuZG9jcmluZSBOdW1vcnM8L3RpdGxlPjxzZWN vbmRhcnktdGl0 bGU+U2NpZW5jZTwvc2Vjb25kYXJ5LXRpdGxlPjxhbHQtdGl0bGU+U2NpZW5jZTw vYWx0LXRpdGxl PjwvdGl0bGVzPjxwZXJpb2RpY2FsPjxmdWxsLXRpdGxlPlNjaWVuY2U8L2Z1bGwtd Gl0bGU+PGFi YnItMT5TY211bmN1PC9hYmJyLTE+PC9wZXJpb2RpY2FsPjxhbHQtcGVyaW9kaWNh bD48ZnVsbC10 aXRsZT5TY211bmN1PC9mdWxsLXRpdGxlPjxhYmJyLTE+U2NpZW5jZTwvYWJiciOx PjwvYWx0LXBI cmlvZGljYWw+PHBhZ2VzPjExOTktMjAzPC9wYWdlcz48dm9sdWllPjMzMTwvdm9 sdWllPjxudWli ZXI+NjAyMTwvbnVtYmVyPjxrZX13b3Jkcz48a2V5d29yZD5BZGFwdG9yIFByb3Rla W5zLCBTaWdu YWwgVHJhbnNkdWNpbmcvKmdlbmV0aWNzPC9rZX13b3JkPjxrZX13b3JkPkNhcmN pbm9tYSwgUGFu Y3J1YXRpYyBEdWN0YWwvZ2VuZXRpY3M8L2tleXdvcmQ+PGtleXdvcmQ+Q2hyb 21hdGluIEFzc2Vt Ymx5IGFuZCBEaXNhc3NlbWJseS9nZW51dGljczwva2V5d29yZD48a2V5d29yZD5ET kEgSGVsaWNh c2VzLypnZW5ldGljczwva2V5d29yZD48a2V5d29yZD5HZW5lcywgVHVtb3IgU3Vwc HJlc3Nvcjwv a2V5d29yZD48a2V5d29yZD5IdW1hbnM8L2tleXdvcmQ+PGtleXdvcmQ+Kk11dGFOa W9uPC9rZX13 b3JkPjxrZX13b3JkPk51dXJvZW5kb2NyaW51IFR1bW9ycy8qZ2VuZXRpY3MvbWVOY WJvbGlzbTwv a2V5d29yZD48a2V5d29yZD50dWNsZWFyIFByb3R1aW5zLypnZW5ldGljczwva2V5d 29yZD48a2V5 d29yZD5QVEVOIFBob3NwaG9oeWRyb2xhc2UvZ2VuZXRpY3M8L2tleXdvcmQ+PG tleXdvcmQ+UGFu Y3JlYXRpYyBOZW9wbGFzbXMvKmdlbmV0aWNzL21ldGFib2xpc208L2tleXdvcmQ+PGtleXdvcmQ+UGhvc3BoYXRpZHlsaW5vc210b2wgMy1LaW5hc2VzL2dlbmV0aWNzPC9rZX13b3Jk PjxrZX13b3Jk PlByb2dub3Npczwva2V5d29yZD48a2V5d29yZD5Qcm90by1PbmNvZ2VuZSBQcm90 ZWlucy8qZ2Vu ZXRpY3M8L2tleXdvcmQ+PGtleXdvcmQ+U2VxdWVuY2UgQW5hbHlzaXMsIEROQ Twva2V5d29yZD48 a2V5d29yZD5TaWduYWwgVHJhbnNkdWNOaW9uL2dlbmV0aWNzPC9rZX13b3JkPjx rZX13b3JkPlRP UiBTZXJpbmUtVGhyZW9uaW51IEtpbmFzZXMvZ2VuZXRpY3MvKm11dGFib2xpc20 8L2tleXdvcmQ+PGtleXdvcmQ+VHVtb3IgU3VwcHJlc3NvciBQcm90ZWlucy9nZW5ldGljczwva2V5d29 yZD48L2tl eXdvcmRzPjxkYXRlcz48eWVhcj4yMDExPC95ZWFyPjxwdWItZGF0ZXM+PGRhdG U+TWFyIDQ8L2Rh dGU+PC9wdWItZGF0ZXM+PC9kYXRlcz48aXNibj4xMDk1 LTkyMDMgKEVsZWNO cm9uaWMpJiN4RDsw MDM2LTgwNzUgKExpbmtpbmcpPC9pc2JuPjxhY2Nlc3Npb24tbnVtPjIxMjUyMzE1P C9hY2Nlc3Np b24tbnVtPjxl cmxzPjxyZWxhdGVkLXVybHM+PHVybD5odHRwOi8vd3d3Lm5jYmku bmxtLm5paC5n b3YvcHVibWVkLzIxMjUyMzE1PC91cmw+PC9yZWxhdGVkLXVybHM+PC91cmxzP jxjdXN0b20yPjMx NDQOOTY8L2N1c3RvbTI+PGVsZWN0cm9uaWMtcmVzb3VyY2UtbnVtPjEwLjExMj Yvc2NpZW5jZS4x MjAwNjA5PC9lbGVjdHJvbmljLXJlc291cmNlLW51bT48L3JlY29yZD48LONpdGU+P C9FbmROb3Rl PgB=20. For a subset of cases, plasma samples were obtained at multiple time points after surgery.

### Flow-Sorting of Aneuploid Nuclei

Flow sorting of tumor nuclei: Individual biopsies were minced with scalpel blades in a petri dish (35×100 mm) in NST buffer (146 mM NaCl, 10 mM Tris-HCl, pH 7.5, 1 mM CaCl₂), 0.5 mM MgSO4, 21 mM MgCl2, 0.05% bovine serum albumin, 0.2% Nonidet P40 (Sigma)) with 4,6-diamindino-2-phenylindole (DAPI; 10 g/ml; Boehringer) according to published protocols (Ruiz, Lenkiewicz et al. 2011). The nuclei were then disaggregated with a 25-gauge needle, and subsequently filtered through a 40-μm mesh filter immediately before analyses on an Influx cytometer (Becton-Dickinson, San Jose CA), with ultraviolet excitation and DAPI emission collected at >450 nm. We sorted all diploid, aneuploid, and tetraploid fractions from each sample. DNA content and cell cycle were analyzed using the software program MultiCycle (Phoenix Flow Systems, San Diego, CA).

### Sample Preparation and Next-Generation Sequencing

Sample preparation, library construction, exome and targeted capture, next generation sequencing, and bioinformatics analyses of tumor and normal samples were performed as previously described. In brief, DNA was extracted from frozen or formalin-fixed paraffin embedded (FFPE) tissue, along with matched blood or saliva samples using the Qiagen DNA FFPE tissue kit or Qiagen DNA blood mini kit (Qiagen, CA). Genomic DNA from tumor and normal samples were fragmented and used for Illumina TruSeq library construction (Illumina, San Diego, CA) according to the manufacturer's instructions or as previously describedPEVuZE5vdGU+PENpdGU+PEF1dGhvcj5TYXVzZW48LOF1dGhvcj48WW Vhcj4yMDEzPC9ZZWFyPjxS ZWNOdW0+MTwvUmVjTnVtPjxEaXNwbGF5VGV4dD48c3R5bGUgZmFjZT0ic3Vw ZXJzY3JpcHQiPjE3 PC9zdHlsZT48L0Rpc3BsYX1UZXhOPjxyZWNvcmQ+PHJlYyludWliZXI+MTwvcmV jLW5lbWJlcj48 Zm9yZWlnbilrZXlzPjxrZXkgYXBwPSJFTiIgZGItaWQ9InNheGV3eDVhZzBzdGE5Z XR6NThwMnN1 ZD13eHBzNXBzMnBOMiI+MTwva2V5PjwvZm9yZWlnbilrZXlzPjxyZWYtdHlwZSBu YWllPSJKb3Vy bmFsIEFydGljbGUiPjE3PC9yZWYtdHlwZT48Y29udHJpYnVOb3JzPjxhdXRob3JzPjxh dXRob3I+U2F1c2VuLCBNLjwvYXV0aG9yPjxhdXRob3I+TGVhcnksIFIuIEouPC9hdXRob3I+P GF1dGhvcj5K b251cywgUy48L2F1dGhvcj48YXV0aG9yPld1LCBKLjwvYXV0aG9yPjxhdXRob3I+U mV5bm9sZHMs IEMuIFAuPC9hdXRob3I+PGF1dGhvcj5MaXUsIFguPC9hdXRob3I+PGF1dGhvcj5CbG Fja2ZvcmQs IEEuPC9hdXRob3I+PGF1dGhvcj5QYXJtaWdpYW5pLCBHLjwvYXV0aG9yPjxhdXR ob3I+RGlheiwg TC4gQ S4sIEpyLjwvYXV0aG9yPjxhdXRob3I+UGFwYWRvcG91bG9zLCBOLjwvYX V0aG9yPjxhdXRo b3I+Vm9nZWxzdGVpbiwgQi48L2F1dGhvcj48YXV0aG9yPktpbnpsZXIsIEsuIFcuPC9h dXRob3I+PGF1dGhvcj5WZWxjdWxlc2N1LCBWLiBFLjwvYXV0aG9yPjxhdXRob3I+SG9nYXJ 0eSwgTS4gRC48 L2F1dGhvcj48L2F1dGhvcnM+PC9jb250cmlidXRvcnM+PGF1dGgtYWRkcmVzcz5Md WR3aWcgQ2Vu dGVyIGZvciBDYW5jZXIgR2VuZXRpY3MgYW5kIFRoZXJhcGV1dGljcywgVGhlIFN pZG5leSBLaWlt ZWwgQ29tcHJlaGVuc212ZSBDYW5jZXIgQ2VudGVyIGFOIEpvaG5zIEhvcGtpbnMsI EJhbHRpbW9y ZSwgTWFyeWxhbmQsIFVTQS48L2F1dGgtYWRkcmVzcz48dGl0bGVzPjx0aXRsZT5 JbnR1Z3JhdGVk IGdlbm9taWMgYW5hbH1zZXMgaWRlbnRpZnkgQVJJRDFBIGFuZCBBUk1EMUIgY WxOZXJhdGlvbnMg aW4gdGhlIGNoaWxkaG9vZCBjYW5jZXIgbmV1cm9ibGFzdG9tYTwvdGl0bGU+PHN 1Y29uZGFyeS10 aXRsZT5OYXQgR2VuZXQ8L3N1Y29uZGFyeS1OaXRsZT48YWx0LXRpdGxlPk5hdH VyZSBnZW5ldGlj czwvYWx0LXRpdGxlPjwvdGl0bGVzPjxwZXJpb2RpY2FsPjxmdWxsLXRpdGxlPk5hd CBHZW5ldDwv ZnVsbC1OaXRsZT48YWJici0xPk5hdHVyZSBnZW5ldGljczwvYWJici0xPjwvcGVyaW 9kaWNhbD48 YWx0LXBlcmlvZGljYWw+PGZ1bGwtdGl0bGU+TmFOIEdlbmVOPC9mdWxsLXRpd GxlPjxhYmJyLTE+TmF0dXJIIGdlbmV0aWNzPC9hYmJyLTE+PC9hbHQtcGVyaW9kaWNhbD48cGFnZX M+MTItNzwvcGFn ZXM+PHZvbHVtZT40NTwvdm9sdWllPjxudW1iZXI+MTwvbnVtYmVyPjxrZX13b3Jk cz48a2V5d29y ZD5DZWxsIExpbmUsIFR1bW9yPC9rZX13b3JkPjxrZX13b3JkPkNoaWxkPC9rZX13b3J kPjxrZX13 b3JkPkROQSBDb3B5IE51bWJlciBWYXJpYXRpb25zPC9rZX13b3JkPjxrZX13b3JkPkR OQS1CaW5k aW5nIFByb3RlaW5zLypnZW5ldGljczwva2V5d29yZD48a2V5d29yZD5FeG9tZTwva2 V5d29yZD48a2V5d29yZD5HZW51IEV4cHJlc3Npb24gUHJvZmlsaW5nPC9rZX13b3JkPjxrZX13b3Jk PkdlbmUg T3JkZXI8L2tleXdvcmQ+PGtleXdvcmQ+R2Vub21pY3M8L2tleXdvcmQ+PGtleXdvcm Q+SHVtYW5z PC9rZX13b3JkPjxrZX13b3JkPipNdXRhdGlvbjwva2V5d29yZD48a2V5d29yZD5OZXVy b2JsYXN0 b21hLypnZW5ldGljcy9tb3JOYWxpdHk8L2tleXdvcmQ+PGtleXdvcmQ+TnVjbGVhciB Qcm90ZWlu cy8qZ2VuZXRpY3M8L2tleXdvcmQ+PGtleXdvcmQ+U2VxdWVuY2UgQW5hbHlzaX MsIEROQTwva2V5 d29yZD48a2V5d29yZD5UcmFuc2NyaXBOaW9uIEZhY3RvcnMvKmdlbmV0aWNzPC 9rZX13b3JkPjxr ZX13b3JkP1R1bW9yIE1hcmtlcnMsIEJpb2xvZ2ljYWwvZ2VuZXRpY3M8L2tleXdvcmQ+PC9rZX13 b3Jkcz48ZGF0ZXM+PHIIYXI+MjAxMzwveWVhcj48cHViLWRhdGVzPjxkYXRlPkph bjwvZGF0ZT48 L3B1Yi1kYXR1cz48L2RhdGVzPjxpc2JuPjE1NDYtMTcxOCAoRWx1Y3Ryb25pYykmI 3hEOzEwNjEt NDAzNiAoTGlua2luZyk8L2lzYm4+PGFjY2Vzc2lvbiludWO+MjMyMDIxMjg8L2FjY 2Vzc2lvbilu dW0+PHVybHM+PHJbGFOZWQtdXJscz48dXJsPmhOdHA6Ly93d3cubmNiaS5ubGOu bmloLmdvdi9w dWJtZWQvMjMyMDIxMjg8L3VybD48L3J1bGFOZWQtdXJscz48L3VybHM+PGN1c3 RvbTI+MzU1Nzk1 OTwvY3VzdG9tMj48ZWxlY3Ryb25pYy lyZXNvdXJjZSludWO+MTAuMTAzOC9uZ y4yNDkzPC9lbGVj dHJvbmljLXJlc291cmN1LW51bT48L3J1Y29yZD48LONpdGU+PC9FbmROb3R1PgB=PEVuZE5vdGU+PENpdGU+PEF1dGhvcj5TYXVzZW48LOF1dGhvcj48WWVhcj4yM DEzPC9ZZWFyPjxS ZWNOdW0+MTwvUmVj TnVtPjxEaXNwbGF5VGV4dD48c3R5bGUgZmFjZT0ic3Vw ZXJzY3JpcHQiPjE3 PC9zdHlsZT48L0Rpc3BsYX1UZXhOPjxyZWNvcmQ+PHJ1Yy 1udW1iZXI+MTwvcmV jLW5lbWJlcj48 Zm9yZWlnbi1rZXlzPjxrZXkgYXBwPSJFTiIgZGItaWQ9InNheGV3eDVhZzBzdGE5Z XR6NThwMnN1 ZD13eHBzNXBzMnBOMiI+MTwva2V5PjwvZm9yZWlnbi1rZXlzPjxyZWYtdH1wZSBu YW11PSJKb3Vy bmFsIEFydGljbGUiPjE3PC9yZWYtdH1wZT48Y29udHJpYnVOb3JzPjxhdXRob3JzPjxh dXRob3I+U2F1c2VuLCBNLjwvYXV0aG9yPjxhdXRob3I+TGVhcnksIFIuIEouPC9hdXRob3I+P GF1dGhvcj5K b251cywgUy48L2F1dGhvcj48YXV0aG9yPld1LCBKLjwvYXV0aG9yPjxhdXRob3I+U mV5bm9sZHMs IEMuIFAuPC9hdXRob3I+PGF1dGhvcj5MaXUsIFguPC9hdXRob3I+PGF1dGhvcj5CbG Fja2ZvcmQs IEEuPC9hdXRob3I+PGF1dGhvcj5QYXJtaWdpYW5pLCBHLjwvYXV0aG9yPjxhdXR ob3I+RGlheiwg TC4gQ S4sIEpyLjwvYXV0aG9yPjxhdXRob3I+UGFwYWRvcG91bG9zLCBOLjwvYX V0aG9yPjxhdXRo b3I+Vm9nZWxzdGVpbiwgQi48L2F1dGhvcj48YXV0aG9yPktpbnpsZXIsIEsuIFcuPC9h dXRob3I+PGF1dGhvcj5WZWxjdWxlc2N1LCBWLiBFLjwvYXV0aG9yPjxhdXRob3I+SG9nYXJ 0eSwgTS4gRC48 L2F1dGhvcj48L2F1dGhvcnM+PC9jb250cmlidXRvcnM+PGF1dGgtYWRkcmVzcz5Md WR3aWcgQ2Vu dGVyIGZvciBDYW5jZXIgR2VuZXRpY3MgYW5kIFRoZXJhcGV1dGljcywgVGhlIFN pZG5leSBLaWlt ZWwgQ29tcHJlaGVuc212ZSBDYW5jZXIgQ2VudGVyIGFOIEpvaG5zIEhvcGtpbnMsI EJhbHRpbW9y ZSwgTWFyeWxhbmQsIFVTQS48L2F1dGgtYWRkcmVzcz48dGl0bGVzPjx0aXRsZT5 JbnR1Z3JhdGVk IGdlbm9taWMgYW5hbH1zZXMgaWRlbnRpZnkgQVJJRDFBIGFuZCBBUklEMUIgY WxOZXJhdGlvbnMg aW4gdGhlIGNoaWxkaG9vZCBjYW5jZXIgbmV1cm9ibGFzdG9tYTwvdGl0bGU+PHN IY29uZGFyeS10 aXRsZT50YXQgR2VuZXQ8L3N1Y29uZGFyeS1OaXRsZT48YWx0LXRpdGxlPk5hdH VyZSBnZW5ldGlj czwvYWx0LXRpdGxlPjwvdGl0bGVzPjxwZXJpb2RpY2FsPjxmdWxsLXRpdGxlPk5hd CBHZW5ldDwv ZnVsbC1OaXRsZT48YWJici0xPk5hdHVyZSBnZW5ldGljczwvYWJici0xPjwvcGVyaW 9kaWNhbD48 YWx0LXBlcmlvZGljYWw+PGZ1bGwtdGl0bGU+TmF0IEdlbmVOPC9mdWxsLXRpd GxlPjxhYmJyLTE+TmF0dXJIIGdlbmV0aWNzPC9hYmJyLTE+PC9hbHQtcGVyaW9kaWNhbD48cGFnZX M+MTItNzwvcGFn ZXM+PHZvbHVtZT40NTwvdm9sdW1lPjxudW1iZXI+MTwvbnVtYmVyPjxrZX13b3Jk cz48a2V5d29y ZD5DZWxsIExpbmUsIFR1bW9yPC9rZX13b3JkPjxrZX13b3JkPkNoaWxkPC9rZX13b3J kPjxrZX13 b3JkPkROQSBDb3B5IE51bWJlciBWYXJpYXRpb25zPC9rZX13b3JkPjxrZX13b3JkPkR OQS1CaW5k aW5nIFByb3RlaW5zLypnZW5ldGljczwva2V5d29yZD48a2V5d29yZD5FeG9tZTwva2 V5d29yZD48 a2V5d29yZD5HZW51IEV4cHJlc3Npb24gUHJvZmlsaW5nPC9rZX13b3JkPjxrZX13b3Jk PkdlbmUg T3JkZXI8L2tleXdvcmQ+PGtleXdvcmQ+R2Vub21pY3M8L2tleXdvcmQ+PGtleXdvcm Q+SHVtYW5z PC9rZX13b3JkPjxrZX13b3JkPipNdXRhdGlvbjwva2V5d29yZD48a2V5d29yZD5OZXVy b2JsYXN0 b21hLypnZW5ldGljcy9tb3JOYWxpdHk8L2tleXdvcmQ+PGtleXdvcmQ+TnVjbGVhciB Qcm90ZWlu cy8qZ2VuZXRpY3M8L2tleXdvcmQ+PGtleXdvcmQ+U2VxdWVuY2UgQW5hbHlzaX MsIEROQTwva2V5 d29yZD48a2V5d29yZD5UcmFuc2NyaXBOaW9uIEZhY3RvcnMvKmdlbmV0aWNzPC 9rZX13b3JkPjxr ZX13b3JkP1R1bW9yIE1hcmtlcnMsIEJpb2xvZ2ljYWwvZ2VuZXRpY3M8L2tleXdvcmQ+PC9rZX13 b3Jkcz48ZGF0ZXM+PHIIYXI+MjAxMzwveWVhcj48cHViLWRhdGVzPjxkYXRlPkph bjwvZGF0ZT48 L3B1Yi1kYXR1cz48L2RhdGVzPjxpc2JuPjE1NDYtMTcxOCAoRWx1Y3Ryb25pYykmI 3hEOzEwNjEt NDAzNiAoTGlua2luZyk8L2lzYm4+PGFjY2Vzc2lvbiludWO+MjMyMDIxMjg8L2FjY 2Vzc2lvbilu dW0+PHVybHM+PHJ1bGFOZWQtdXJscz48dXJsPmhOdHA6Ly93d3cubmNiaS5ubGOu bmloLmdvdi9w dWJtZWQvMjMyMDIxMjg8L3VybD48L3J1bGFOZWQtdXJscz48L3VybHM+PGN1c3 RvbTI+MzU1Nzk1 OTwvY3VzdG9tMj48ZWxlY3Ryb25pYy1yZXNvdXJjZS1udW0+MTAuMTAzOC9uZ y4yNDkzPC9lbGVj dHJvbmljLXJlc291cmN1LW51bT48L3J1Y29yZD48LONpdGU+PC9FbmROb3R1PgB=17. Briefly, 50 nanograms (ng)-3 micrograms (μg) of genomic DNA in 100 microliters (μl) of TE was fragmented in a Covaris sonicator (Covaris, Woburn, MA) to a size of 150-450 bp. To remove fragments smaller than 150 bp, DNA was purified using Agencourt AMPure XP beads (Beckman Coulter, IN) in a ratio of 1.0 to 0.9 of PCR product to beads twice and washed using 70% ethanol per the manufacturer's instructions. Purified, fragmented DNA was mixed with 36 μl of H2O, 10 μl of End Repair Reaction Buffer, 5 μl of End Repair Enzyme Mix (cat #E6050, NEB, Ipswich, MA). The 100 μl end-repair mixture was incubated at 20° C. for 30 min, and purified using Agencourt AMPure XP beads (Beckman Coulter, IN) in a ratio of 1.0 to 1.25 of PCR product to beads and washed using 70% ethanol per the manufacturer's instructions. To A-tail, 42 μl of end-repaired DNA was mixed with 5 μl of IOX dA Tailing Reaction Buffer and 3 μl of Klenow (exo-)(cat #E6053, NEB, Ipswich, MA). The 50 μl mixture was incubated at 37° C. for 30 min and purified using Agencourt AMPure XP beads (Beckman Coulter, IN) in a ratio of 1.0 to 1.0 of PCR product to beads and washed using 70% ethanol per the manufacturer's instructions. For adaptor ligation, 25 μl of A-tailed DNA was mixed with 6.7 μl of H2O, 3.3 μl of PE-adaptor (Illumina), 10 μl of 5×Ligation buffer and 5 μl of Quick T4 DNA ligase (cat #E6056, NEB, Ipswich, MA). The ligation mixture was incubated at 20° C. for 15 min and purified using Agencourt AMPure XP beads (Beckman Coulter, IN) in a ratio of 1.0 to 0.95 and 1.0 of PCR product to beads twice and washed using 70% ethanol per the manufacturer's instructions. To obtain an amplified library, twelve PCRs of 25 μl each were set up, each including 15.5 μl of H2O, 5 μl of 5×Phusion HF buffer, 0.5 μl of a dNTP mix containing 10 mM of each dNTP, 1.25 μl of DMSO, 0.25 μl of Illumina PE primer #1, 0.25 μl of Illumina PE primer #2, 0.25 μl of Hotstart Phusion polymerase, and 2 μl of the DNA. The PCR program used was: 98° C. for 2 minutes; 12 cycles of 98° C. for 15 seconds, 65° C. for 30 seconds, 72° C. for 30 seconds; and 72° C. for 5 min. DNA was purified using Agencourt AMPure XP beads (Beckman Coulter, IN) in a ratio of 1.0 to 1.0 of PCR product to beads and washed using 70% ethanol per the manufacturer's instructions. Exonic or targeted regions were captured in solution using the Agilent SureSelect v.4 kit or a custom targeted panel for the 111 genes of interest according to the manufacturer's instructions (Agilent, Santa Clara, CA). The captured library was then purified with a Qiagen MinElute column purification kit and eluted in 17 μl of 70° C. EB to obtain 15 μl of captured DNA library. (5) The captured DNA library was amplified in the following way: Eight 30 uL PCR reactions each containing 19 μl of H2O, 6 μl of 5×Phusion HF buffer, 0.6 μl of 10 mM dNTP, 1.5 μl of DMSO, 0.30 μl of Illumina PE primer #1, 0.30 μl of Illumina PE primer #2, 0.30 μl of Hotstart Phusion polymerase, and 2 μl of captured exome library were set up. The PCR program used was: 98° C. for 30 seconds; 14 cycles (exome) or 16 cycles (targeted) of 98° C. for 10 seconds, 65° C. for 30 seconds, 72° C. for 30 seconds; and 72° C. for 5 min. To purify PCR products, a NucleoSpin Extract II purification kit (Macherey-Nagel, PA) was used following the manufacturer's instructions. Paired-end sequencing, resulting in 100 bases from each end of the fragments for exome libraries and 150 bases from each end of the fragment for targeted libraries, was performed using Illumina HiSeq 2000/2500 and Illumina MiSeq instrumentation (Illumina, San Diego, CA).

### Primary Processing of Next-Generation Sequencing Data and Identification of Putative Somatic Mutations

Somatic mutations were identified using VariantDx custom software for identifying mutations in matched tumor and normal samples through Personal Genome Diagnostics (Baltimore, MD). Prior to mutation calling, primary processing of sequence data for both tumor and normal samples were performed using Illumina CASAVA software (v1.8), including masking of adapter sequences. Sequence reads were aligned against the human reference genome (version hg18) using ELAND with additional realignment of select regions using the Needleman-Wunsch method 18. Candidate somatic mutations, consisting of point mutations, insertions, and deletions were then identified using VariantDx across the either the whole exome or regions of interest. VariantDx examines sequence alignments of tumor samples against a matched normal while applying filters to exclude alignment and sequencing artifacts. In brief, an alignment filter was applied to exclude quality failed reads, unpaired reads, and poorly mapped reads in the tumor. A base quality filter was applied to limit inclusion of bases with reported phred quality score >30 for the tumor and >20 for the normal. A mutation in the tumor was identified as a candidate somatic mutation only when (i) distinct paired reads contained the mutation in the tumor; (ii) the number of distinct paired reads containing a particular mutation in the tumor was at least 2% of the total distinct read pairs for targeted analyses and 10% of read pairs for exome and (iii) the mismatched base was not present in >1% of the reads in the matched normal sample as well as not present in a custom database of common germline variants derived from dbSNP and (iv) the position was covered in both the tumor and normal. Mutations arising from misplaced genome alignments, including paralogous sequences, were identified and excluded by searching the reference genome. Candidate somatic mutations were further filtered based on gene annotation to identify those occurring in protein coding regions. Functional consequences were predicted using snpEff and a custom database of CCDS, RefSeq and Ensembl annotations using the latest transcript versions available on hgl8 from UCSC (see URL genome.ucsc.edu). Predictions were ordered to prefer transcripts with canonical start and stop codons and CCDS or Refseq transcripts over Ensembl when available. Finally mutations were filtered to exclude intronic and silent changes, while retaining mutations resulting in missense mutations, nonsense mutations, frameshifts, or splice site alterations. A manual visual inspection step was used to further remove artifactual changes.

Identification of Putative Somatic Mutations without Matched Normal Sample

For the identification of putative somatic mutations without a matched normal, additional filters were applied. Firstly, mutations present in an unmatched normal sample, sequenced to a similar coverage and on the same platform as the matched normal, were removed. Second, alterations reported in the 1000 Genomes project, present in >1% of the population or listed as Common in dbSNP138 were filtered.

### Clinical Actionability Analyses

We selected 200 well characterized genes with potential clinical significance and assessed the level of evidence for clinical actionability in three ways. Firstly, we determined which of the genes were associated with FDA-approved therapies (see URL fda.gov/Drugs/). Secondly, we carried out a literature search to identify published prospective clinical studies pertaining to genomic alterations of each gene and their association with outcome for cancer patients. Genes that served as targets for specific agents or were predictors of response or resistance to cancer therapies when mutated were considered actionable. Thirdly, we identified clinical trials (see URL clinicaltrials.gov) that specified altered genes within the inclusion criteria and were actively recruiting patients in August 2014. In all cases, the tumor type relevant to the FDA approval or studied in the clinical trials was determined to allow the clinical information to be matched to the mutational data by both gene and cancer type.

### Statistical Analyses for Clinical and Genetic Data

Unpaired T-test and chi-square were employed to compare mutation status of MLL genes among different groups with different clinical and pathological characteristics. Curves for overall survival and progression free survival (calculated as the time from diagnosis to disease progression) were constructed using the Kaplan-Meier method and compared between groups using the log-rank test. Cox proportional hazards regression analysis was used to determine which independent factors jointly had a significant impact on overall survival. All p values were based on 2-sided testing and differences were considered significant at p<0.05. Passenger probabilities were calculated using the binomial test adjusted for gene sizes and corrected for multiple comparisons. Genes which were recurrently mutated within the comprehensive exome analysis (>2 cases) were considered. Statistical analyses of clinical and genetic features were performed with SPSS version 22 for windows, while conservation of specific genomic positions were evaluated using phyloP softwarePEVuZE5vdGU+PENpdGU+PEF1dGhvcj5Db29wZXI8L0F1dGhvcj48WWVhc j4yMDA1PC9ZZWFyPjxS ZWNOdW0+MzwvUmVjTnVtPjxEaXNwbGF5VGV4dD48c3R5bGUgZmFjZT0ic3Vw ZXJzY3JpcHQiPjIz PC9zdHlsZT48L0Rpc3BsYX1UZXhOPjxyZWNvcmQ+PHJlYy1udW1iZXI+MzwvcmVj LW5lbWJlcj48 Zm9yZWlnbi1rZXlzPjxrZXkgYXBwPSJFTiIgZGItaWQ9IndkdmQyeDJ3NDJkOXgzZX h4YW54NWRw ZXJkZWRwdHRhMnhmeCI+Mzwva2V5PjwvZm9yZWlnbi1rZXlzPjxyZWYtdHlwZSB uYWllPSJKb3Vy bmFsIEFydGljbGUiPjE3PC9yZWYtdHlwZT48Y29udHJpYnVOb3JzPjxhdXRob3JzPjxh dXRob3I+Q29vcGVyLCBHLiBNLjwvYXV0aG9yPjxhdXRob3I+U3RvbmUsIEUuIEEuPC9hdXR ob3I+PGF1dGhv cj5Bc2ltZW5vcywgRy48L2F1dGhvcj48YXV0aG9yPk5pc2MgQ29tcGFyYXRpdmUgU 2VxdWVuY21u ZyBQcm9ncmFtPC9hdXRob3I+PGF1dGhvcj5HcmVlbiwgRS4gRC48L2F1dGhvcj48Y XV0aG9yPkJh dHpvZ2xvdSwgUy48L2F1dGhvcj48YXV0aG9yP1NpZG93LCBBLjwvYXV0aG9yPjwv YXV0aG9ycz48 L2NvbnRyaWJ1dG9ycz48YXVOaC1hZGRyZXNzPkRlcGFydG11bnQgb2YgR2VuZXR pY3MsIFNOYW5m b3JkIFVuaXZlcnNpdHksIFNOYW5mb3JkLCBDYWxpZm9ybmlhIDkOMzA1LCBVUO EuPC9hdXRoLWFk ZHJlc3M+PHRpdGxlcz48dGl0bGU+RGlzdHJpYnVOaW9uIGFuZCBpbnRlbnNpdHkgb 2YgY29uc3Ry YW1udCBpbiBtYW1tYWxpYW4gZ2Vub21pYyBzZXF1ZW5jZTwvdGl0bGU+PHN1Y 29uZGFyeS1OaXRs ZT5HZW5vbWUgUmVzPC9zZWNvbmRhcnktdGl0bGU+PGFsdC1OaXRsZT5HZW5v bWUgcmVzZWFyY2g8 L2FsdC1OaXRsZT48L3RpdGxlcz48cGVyaW9kaWNhbD48ZnVsbC1OaXRsZT5HZW5 vbWUgUmVzPC9m dWxsLXRpdGxlPjxhYmJyLTE+R2Vub211IHJ1c2VhcmNoPC9hYmJyLTE+PC9wZXJp b2RpY2FsPjxh bHQtcGVyaW9kaWNhbD48ZnVsbC1OaXRsZT5HZW5vbWUgUmVzPC9mdWxsLXR pdGxlPjxhYmJyLTE+R2Vub211IHJ1c2VhcmNoPC9hYmJyLTE+PC9hbHQtcGVyaW9kaWNhbD48cGFnZX M+OTAxLTEzPC9w YWdlcz48dm9sdWllPjE1PC92b2x1bWU+PG51bWJlcj43PC9udW1iZXI+PGtleXdvcm RzPjxrZX13 b3JkPkFuaW1hbHM8L2tleXdvcmQ+PGtleXdvcmQ+QmFzZSBTZXF1ZW5jZTwva2V 5d29yZD48a2V5 d29yZD5Db21wdXRhdGlvbmFslEJpb2xvZ3kvbWV0aG9kczwva2V5d29yZD48a2V5d2 9yZD5Db25z ZXJ2ZWQgU2VxdWVuY2U8L2tleXdvcmQ+PGtleXdvcmQ+Q3lzdGljIEZpYnJvc2lzIF RyYW5zbWVt YnJhbmUgQ29uZHVjdGFuY2UgUmVndWxhdG9yPC9rZX13b3JkPjxrZX13b3JkPipFd m9sdXRpb24s IE l1vbGVjdWxhcjwva2V5d29yZD48a2V5d29yZD5HZW51IENvbXBvbmVudHM8L2tle XdvcmQ+PGtl eXdvcmQ+R2VuZSBFeHByZXNzaW9uIFByb2ZpbGluZy9tZXRob2RzPC9rZX13b3JkP jxrZX13b3Jk PipHZW5vbWU8L2tleXdvcmQ+PGtleXdvcmQ+SHVtYW5zPC9rZX13b3JkPjxrZX13b3 JkPkludGVy c3B1cnN1ZCBSZXB1dG10aXZ1IFN1cXV1bmNlczwva2V5d29yZD48a2V5d29yZD5NYW 1tYWxzLypn ZW5ldGljczwva2V5d29yZD48a2V5d29yZD5Nb2xlY3VsYXIgU2VxdWVuY2UgRGFO YTwva2V5d29y ZD48a2V5d29yZD5TZW5zaXRpdmIOeSBhbmQgU3BlY21maWNpdHk8L2tleXdvcmQ+PGtleXdvcmQ+U2VxdWVuY2UgQWxpZ25tZW5OPC9rZX13b3JkPjxrZX13b3JkPNlcXVlbmNIIEFuY Wx5c21zLCBE TkE8L2tleXdvcmQ+PGtleXdvcmQ+U2VxdWVuY2UgSG9tb2xvZ3ksIE51Y2xlaWMg QWNpZDwva2V5 d29yZD48L2tleXdvcmRzPjxkYXRlcz48eWVhcj4yMDA1PC95ZWFyPjxwdWItZGFOZ XM+PGRhdGU+SnVsPC9kYXR1PjwvcHViLWRhdGVzPjwvZGF0ZXM+PG1zYm4+MTA4OC05MDUx IChQcmludCkmI3 hE OzEwODgtOTA1MSAoTGlua2luZyk8L2lzYm4+PGFjY2Vzc2lvbi1udW0+MTU5NjUw Mjc8L2FjY2Vz c2lvbiludWO+PHVybHM+PHJ1bGFOZWQtdXJscz48dXJsPmhOdHA6Ly93d3cubmNiaS 5ubGOubmlo Lmdvdi9wdWJtZWQvMTU5NjUwMjc8L3VybD48L3J1bGFOZWQtdXJscz48L3VybH M+PGN1c3RvbTI+MTE3MjAzNDwvY3VzdG9tMj48ZWxlY3Ryb25pYy1yZXNvdXJjZSiudWO+MTAuM TEwMS9nci4zNTc3 NDA1PC91bGVjdHJvbmljLXJlc291cmN1LW51bT48L3J1Y29yZD48LONpdGU+PC9Fb mROb3R1PgB=PEVuZE5vdGU+PENpdGU+PEF1dGhvcj5Db29wZXI8L0F1dGhvcj48WWVhcj4yMD A1PC9ZZWFyPjxS ZWNOdW0+MzwvUmVj TnVtPjxEaXNwbGF5VGV4dD48c3R5bGUgZmFjZT0ic3Vw ZXJzY3JpcHQiPjIz PC9zdHlsZT48L0Rpc3BsYX1UZXhOPjxyZWNvcmQ+PHJ1Yy 1udW1iZXI+MzwvcmVj LW5lbWJlcj48 Zm9yZWlnbi1rZXlzPjxrZXkgYXBwPSJFTiIgZGItaWQ9IndkdmQyeDJ3NDJkOXgzZX h4YW54NWRw ZXJkZWRwdHRhMnhmeCI+Mzwva2V5PjwvZm9yZWlnbi1rZXlzPjxyZWYtdH1wZSB uYW11PSJKb3Vy bmFsIEFydGljbGUiPjE3PC9yZWYtdH1wZT48Y29udHJpYnVOb3JzPjxhdXRob3JzPjxh dXRob3I+Q29vcGVyLCBHLiBNLjwvYXV0aG9yPjxhdXRob3I+U3RvbmUsIEUuIEEuPC9hdXR ob3I+PGF1dGhv cj5Bc2ltZW5vcywgRy48L2F1dGhvcj48YXV0aG9yPk5pc2MgQ29tcGFyYXRpdmUgU 2VxdWVuY21u ZyBQcm9ncmFtPC9hdXRob3I+PGF1dGhvcj5HcmVlbiwgRS4gRC48L2F1dGhvcj48Y XV0aG9yPkJh dHpvZ2xvdSwgUy48L2F1dGhvcj48YXV0aG9yP1NpZG93LCBBLjwvYXV0aG9yPjwv YXV0aG9ycz48 L2NvbnRyaWJ1dG9ycz48YXVOaC1hZGRyZXNzPkRlcGFydG11bnQgb2YgR2VuZXR pY3MsIFNOYW5m b3JkIFVuaXZlcnNpdHksIFNOYW5mb3JkLCBDYWxpZm9ybmlhIDkOMzA1LCBVUO EuPC9hdXRoLWFk ZHJlc3M+PHRpdGxlcz48dGl0bGU+RGlzdHJpYnVOaW9uIGFuZCBpbnRlbnNpdHkgb 2YgY29uc3Ry YW1udCBpbiBtYW1tYWxpYW4gZ2Vub21pYyBzZXF1ZW5jZTwvdGObGU+PHN1Y 29uZGFyeS1OaXRs ZT5HZW5vbWUgUmVzPC9zZWNvbmRhcnktdGl0bGU+PGFsdC1OaXRsZT5HZW5v bWUgcmVzZWFyY2g8 L2FsdC1OaXRsZT48L3RpdGxlcz48cGVyaW9kaWNhbD48ZnVsbC1OaXRsZT5HZW5 vbWUgUmVzPC9m dWxsLXRpdGxlPjxhYmJyLTE+R2Vub211IHJ1c2VhcmNoPC9hYmJyLTE+PC9wZXJp b2RpY2FsPjxh bHQtcGVyaW9kaWNhbD48ZnVsbC1OaXRsZT5HZW5vbWUgUmVzPC9mdWxsLXR pdGxlPjxhYmJyLTE+R2Vub211IHJ1c2VhcmNoPC9hYmJyLTE+PC9hbHQtcGVyaW9kaWNhbD48cGFnZX M+OTAxLTEzPC9w YWdlcz48dm9sdWllPjE1PC92b2x1bWU+PG51bWJlcj43PC9udWliZXI+PGtleXdvcm RzPjxrZX13 b3JkPkFuaW1hbHM8L2tleXdvcmQ+PGtleXdvcmQ+QmFzZSBTZXF1ZW5jZTwva2V 5d29yZD48a2V5 d29yZD5Db21wdXRhdGlvbmFslEJpb2xvZ3kvbWV0aG9kczwva2V5d29yZD48a2V5d2 9yZD5Db25z ZXJ2ZWQgU2VxdWVuY2U8L2tleXdvcmQ+PGtleXdvcmQ+Q3lzdGljIEZpYnJvc2lzIF RyYW5zbWVt YnJhbmUgQ29uZHVjdGFuY2UgUmVndWxhdG9yPC9rZX13b3JkPjxrZX13b3JkPipFd m9sdXRpb24s IE l1vbGVjdWxhcjwva2V5d29yZD48a2V5d29yZD5HZW51IENvbXBvbmVudHM8L2tle XdvcmQ+PGtl eXdvcmQ+R2VuZSBFeHByZXNzaW9uIFByb2ZpbGluZy9tZXRob2RzPC9rZX13b3JkP jxrZX13b3Jk PipHZW5vbWU8L2tleXdvcmQ+PGtleXdvcmQ+SHVtYW5zPC9rZX13b3JkPjxrZX13b3 JkPkludGVy c3B1cnN1ZCBSZXB1dG10aXZ1IFN1cXV1bmNlczwva2V5d29yZD48a2V5d29yZD5NYW 1tYWxzLypn ZW5ldGljczwva2V5d29yZD48a2V5d29yZD5Nb2xlY3VsYXIgU2VxdWVuY2UgRGFO YTwva2V5d29y ZD48a2V5d29yZD5TZW5zaXRpdmIOeSBhbmQgU3BlY21maWNpdHk8L2tleXdvcmQ+PGtleXdvcmQ+U2VxdWVuY2UgQWxpZ25tZW50PC9rZX13b3JkPjxrZX13b3JkP1N1cXV1bmN1IEFuY Wx5c21zLCBE TkE8L2tleXdvcmQ+PGtleXdvcmQ+U2VxdWVuY2UgSG9tb2xvZ3ksIE51Y2xlaWMg QWNpZDwva2V5 d29yZD48L2tleXdvcmRzPjxkYXRlcz48eWVhcj4yMDA1PC95ZWFyPjxwdWItZGFOZ XM+PGRhdGU+SnVsPC9kYXRlPjwvcHViLWRhdGVzPjwvZGF0ZXM+PG1zYm4+MTA4OC05MDUx IChQcmludCkmI3 hE OzEwODgtOTA1MSAoTGlua2luZyk8L2lzYm4+PGFjY2Vzc2lvbiludWO+MTU5NjUw Mjc8L2FjY2Vz c2lvbiludWO+PHVybHM+PHJlbGFOZWQtdXJscz48dXJsPmhOdHA6Ly93d3cubmNiaS 5ubGOubmlo Lmdvdi9wdWJtZWQvMTU5NjUwMjc8L3VybD48L3JlbGFOZWQtdXJscz48L3VybH M+PGNlc3RvbTI+MTE3MjAzNDwvY3VzdG9tMj48ZWxlY3Ryb25pYy1yZXNvdXJjZSiudWO+MTAuM TEwMS9nci4zNTc3 NDA1PC9lbGVjdHJvbmljLXJlc291cmNlLW51bT48L3JlY29yZD48LONpdGU+PC9Fb mROb3RlPgB=23.

### Digital PCR Analyses

KRAS, BRAF, or PIK3CA somatic point mutations were identified through sequencing analysis of tumor tissues. In cases with matched plasma samples, point mutations were detected in the plasma using droplet digital PCR (ddPCR) using the BioRad QX200™ Droplet Digital™ PCR System (Hercules, CA). Briefly, specific ddPCR assays for each point mutation were obtained from BioRad (Hercules, CA) and applied to assess the mutant allele fraction (mutant genomic equivalents/total genomic equivalents). Prior to analysis of each point mutation in the patient plasma sample, a panel of at least 160 normal control analyses were used to confirm the mutation specificity of the assay. Additionally, control samples of wild-type DNA were included in each analysis.

### Example 2

In order to identify genetic alterations that may be related to patient outcome and other clinical characteristics, we performed large-scale genomic analyses of pancreatic adenocarcinomas using two prospectively collected clinical cohorts.

We used next generation sequencing to examine the entire exomes of matched tumor-normal specimens from 24 patients and targeted sequencing to analyze an additional 77 patient tumors. These approaches allowed us to identify sequence changes, including single base and small insertion or deletion mutations, as well as copy number alterations in >20,000 genes in the whole-exome analyses and in 116 specific genes in the targeted analyses (Supplementary Table 1). The pancreatic cancers analyzed were stage II tumors in patients who underwent potentially curative resections. Given the low neoplastic cellularity of pancreatic cancersPEVuZE5vdGU+PENpdGU+PEF1dGhvcj5CaWFua2luPC9BdXRob3I+PFllYXI+MjAxMjwvWWVhcj48 UmVjTnVtPjI8L1JlY051bT48RGlzcGxheVReHQ+PHN0eWxlIGZhY2U9InN1cGVyc2 NyaXBOIj4y PC9zdHlsZT48L0Rpc3BsYXIUZXhOPjxyZWNvcmQ+PHJlYy1udW1iZXI+MjwvcmVj LW5lbWJlcj48 Zm9yZWlnbi1rZXlzPjxrZXkgYXBwPSJFTiIgZGItaWQ9IndkdmQyeDJ3NDJkOXgzZX h4YW54NWRw ZXJkZWRwdHRhMnhmeCI+Mjwva2V5PjwvZm9yZWlnbi1rZXlzPjxyZWYtdHIwZSB uYWllPSJKb3Vy bmFsIEFydGljbGUiPjE3PC9yZWYtdHlwZT48Y29udHJpYnVOb3JzPjxhdXRob3JzPjxh dXRob3I+QmlhbmtpbiwgQS4gVi48L2F1dGhvcj48YXV0aG9yPldhZGRlbGwsIE4uPC9hdXRob3I+PGF1dGhv cj5LYXNzYWhuLCBLLiBTLjwvYXV0aG9yPjxhdXRob3I+R2luZ3JhcywgTS4gQy48 L2F1dGhvcj48 YXV0aG9yPk11dGh1c3dhbXksIEwuIEIuPC9hdXRob3I+PGF1dGhvcj5Kb2hucywgQS 4gTC48L2F1 dGhvcj48YXV0aG9yPk1pbGxlciwgRC4gSy48L2F1dGhvcj48YXV0aG9yPldpbHNvbiw gUC4gSi48 L2F1dGhvcj48YXV0aG9yPlBhdGNoLCBBLiBNLjwvYXV0aG9yPjxhdXRob3I+V3Us IEouPC9hdXRo b3I+PGF1dGhvcj5DaGFuZywgRC4gSy48L2F1dGhvcj48YXV0aG9yPkNvd2xleSwgTS 4gSi48L2F1 dGhvcj48YXV0aG9yPkdhcmRpbmVyLCBCLiBCLjwvYXV0aG9yPjxhdXRob3I+U29u ZywgUy48L2F1 dGhvcj48YXV0aG9yPkhhcmxpd29uZywgSS48L2F1dGhvcj48YXV0aG9yPklkcmlzb2d sdSwgUy48 L2F1dGhvcj48YXV0aG9yPk5vdXJzZSwgQy48L2F1dGhvcj48YXV0aG9yPk5vdXJiY Wtoc2gsIEUu PC9hdXRob3I+PGF1dGhvcj5NYW5uaW5nLCBTLjwvYXV0aG9yPjxhdXRob3I+V2Fu aSwgUy48L2F1 dGhvcj48YXV0aG9yPkdvbmdvcmEsIEOuPC9hdXRob3I+PGF1dGhvcj5QYWppYywg T S48L2F1dGhv cj48YXV0aG9yP1NjYXJsZXROLCBDLiBKLjwvYXV0aG9yPjxhdXRob3I+R21sbCwg QS4gSi48L2F1 dGhvcj48YXV0aG9yPlBpbmhvLCBBLiBWLjwvYXV0aG9yPjxhdXRob3I+Um9vbWF uLCBJLjwvYXVO aG9yPjxhdXRob3I+QW5kZXJzb24sIEOuPC9hdXRob3I+PGF1dGhvcj5Ib2xtZXMsIE8u PC9hdXRo b3I+PGF1dGhvcj5MZW9uYXJkLCBDLjwvYXV0aG9yPjxhdXRob3I+VGF5bG9yLCB ELjwvYXV0aG9y PjxhdXRob3I+V29vZCwgUy48L2F1dGhvcj48YXV0aG9yPlh1LCBRLjwvYXV0aG9yP jxhdXRob3I+Tm9uZXMsIEsuPC9hdXRob3I+PGF1dGhvcj5GaW5rLCBKLiBMLjwvYXV0aG9yPjxh dXRob3I+Q2 hy aXNOLCBBLjwvYXV0aG9yPjxhdXRob3I+QnJ1eG5lciwgVC48L2F1dGhvcj48YXVOa G9yPkNsb29u YW4sIE4uPC9hdXRob3I+PGF1dGhvcj5Lb2xsZSwgRy48L2F1dGhvcj48YXV0aG9yPk 5ld2VsbCwg Ri48L2F1dGhvcj48YXV0aG9yPlBpbmVzZSwgTS48L2F1dGhvcj48YXV0aG9yPk11Y WQsIFIuIFMu PC9hdXRob3I+PGF1dGhvcj5IdW1waHJpcywgSi4gTC48L2F1dGhvcj48YXV0aG9yPkt hcGxhbiwg Vy48L2F1dGhvcj48YXV0aG9yPkpvbmVzLCBNLiBELjwvYXV0aG9yPjxhdXRob3I+Q29sdm1uLCBF LiBLLjwvYXV0aG9yPjxhdXRob3I+TmFncmlhbCwgQS4gT S48L2F1dGhvcj48YXVOa G9yPkhlbXBo cmV5LCBFLiBTLjwvYXV0aG9yPjxhdXRob3I+Q2hvdSwgQS48L2F1dGhvcj48YXV0 aG9yPkNoaW4s IFYuIFQuPC9hdXRob3I+PGF1dGhvcj5DaGFudHJpbGwsIEwuIEEuPC9hdXRob3I+PG F1dGhvcj5N YXdzb24sIEEuPC9hdXRob3I+PGF1dGhvcj5TYW1yYSwgSi4gUy48L2F1dGhvcj48Y XV0aG9yPktl bmNoLCBKLiBHLjwvYXV0aG9yPjxhdXRob3I+TG92ZWxsLCBKLiBBLjwvYXVOa G9yPjxhdXRob3I+RGFseSwgUi4gSi48L2F1dGhvcj48YXV0aG9yPk11cnJldHQsIE4uIEQuPC9hdXRob3I+PGF 1 dGhv cj5Ub29uLCBDLjwvYXV0aG9yPjxhdXRob3I+RXBhcmksIEsuPC9hdXRob3I+PGF1d Ghvcj50Z3V5 ZW4sIE4uIFEuPC9hdXRob3I+PGF1dGhvcj5CYXJib3VyLCBBLjwvYXV0aG9yPjxhd XRob3I+WmVw cywgTi48L2F1dGhvcj48YXV0aG9yPkF 1c3RyYWxpYW4gUGFuY3J1YXRpYyBDYW 5jZXIgR2Vub211 LCBJbm10aWFOaXZ1PC9hdXRob3I+PGF1dGhvcj5LYWtrYXIsIE4uPC9hdXRob3I+PG F1dGhvcj5a aGFvLCBGLjwvYXV0aG9yPjxhdXRob3I+V3UsIFkuIFEuPC9hdXRob3I+PGF1dGhvcj 5XYW5nLCBN LjwvYXV0aG9yPjxhdXRob3I+TXV6bnksIEQuIEOuPC9hdXRob3I+PGF1dGhvcj5GaX NoZXIsIFcu IEUuPC9hdXRob3I+PGF1dGhvcj5CenVuaWNhcmRpLCBGLiBDLjwvYXV0aG9yPjx hdXRob3I+SG9k Z2VzLCBTLiBFLjwvYXV0aG9yPjxhdXRob3I+UmVpZCwgSi4gRy48L2F1dGhvcj48 YXV0aG9yPkRy dW1tb25kLCBKLjwvYXV0aG9yPjxhdXRob3I+Q2hhbmcsIEsuPC9hdXRob3I+PGF1d Ghvcj5IYW4s IFkuPC9hdXRob3I+PGF1dGhvcj5MZXdpcywgTC4gUi48L2F1dGhvcj48YXV0aG9yPk RpbmgsIEgu PC9hdXRob3I+PGF1dGhvcj5CdWhheSwgQy4gSi48L2F1dGhvcj48YXV0aG9yPkJIY2s sIFQuPC9h dXRob3I+PGF1dGhvcj5UaW1tcywgTC48L2F1dGhvcj48YXV0aG9yPlNhbSwgTS48L2 F1dGhvcj48 YXV0aG9yPkJIZ2xleSwgSy48L2F1dGhvcj48YXV0aG9yPkJyb3duLCBBLjwvYXVOa G9yPjxhdXRo b3I+UGFpLCBELjwvYXV0aG9yPjxhdXRob3I+UGFuY2hhbCwgQS48L2F1dGhvcj48 YXV0aG9yPkJ1 Y2huZXIsIE4uPC9hdXRob3I+PGF1dGhvcj5EZSBCb3JqYSwgUi48L2F1dGhvcj48YX V0aG9yPkR1 bnJvY2hlLCBSLiBFLjwvYXV0aG9yPjxhdXRob3I+WXVuZywgQy4gSy48L2F1dGhvc j48YXV0aG9y PINIcnJhLCBTLjwvYXV0aG9yPjxhdXRob3I+T251dHRvLCBOLjwvYXV0aG9yPjxhd XRob3I+TXVr aG9wYWRoeWF5LCBELjwvYXV0aG9yPjxhdXRob3I+VHNhbywgT S4gUy48L2F1dG hvcj48YXV0aG9y PINoYXcsIFAuIEEuPC9hdXRob3I+PGF1dGhvcj5QZXRlcnNlbiwgRy4gTS48L2F1dGh vcj48YXV0aG9yPkdhbGxpbmdlciwgUy48L2F1dGhvcj48YXV0aG9yPkhydWJhbiwgUi4gSC48L2F1 dGhvcj48 YXV0aG9yPk1haXRyYSwgQS48L2F1dGhvcj48YXV0aG9yPklhY29idXppby1Eb25 ha HVILCBDLiBB LjwvYXV0aG9yPjxhdXRob3I+U2NodWxpY2ssIFIuIEQuPC9hdXRob3I+PGF1dGhvcj 5Xb2xmZ2Fu ZywgQy4gTC48L2F1dGhvcj48YXV0aG9yPk1vcmdhbiwgUi4gQS48L2F1dGhvcj48YX V0aG9yPkxh d2xvciwgUi4gVC48L2F1dGhvcj48YXV0aG9yPkNhcGVsbGksIFAuPC9hdXRob3I+PG F1dGhvcj5D b3JibywgVi48L2F1dGhvcj48YXV0aG9yPINjYXJkb25pLCBNLjwvYXV0aG9yPjxhdX Rob3I+VG9y dG9yYSwgRy48L2F1dGhvcj48YXV0aG9yPlRlbXBlcm8sIEOuIEEuPC9hdXRob3I+PG F1dGhvcj5N YW5uLCBLLiBNLjwvYXV0aG9yPjxhdXRob3I+SmVua2lucywgTi4gQ S48L2F1dGhv cj48YXV0aG9y P1B1cmV6LU1hbmNlcmEsIFAuIEEuPC9hdXRob3I+PGF1dGhvcj5BZGFtcywgRC4gSi 48L2F1dGhv cj48YXV0aG9yPkxhcmdhZXNwYWRhLCBELiBBLjwvYXV0aG9yPjxhdXRob3I+V2 Vzc2VscywgTC4g Ri48L2F1dGhvcj48YXV0aG9yP1J1c3QsIEEuIEcuPC9hdXRob3I+PGF1dGhvcj5T dGVp biwgTC4g RC48L2F1dGhvcj48YXV0aG9yP1R1dmVzb24sIEQuIEEuPC9hdXRob3I+PGF1dGhvcj 5Db3B1bGFu ZCwgTi4gRy48L2F1dGhvcj48YXV0aG9yPk11c2dyb3ZILCBFLiBBLjwvYXV0aG9yPj xhdXRob3I+U2NhcnBhLCBBLjwvYXV0aG9yPjxhdXRob3I+RXNobGVtYW4sIEouIFIuPC9hdXRo b3I+PGF1dGhv cj5IdWRzb24sIFQuIEouPC9hdXRob3I+PGF1dGhvcj5TdXRoZXJsYW5kLCBSLiBMLj wvYXV0aG9y PjxhdXRob3I+V2hlZWxlciwgRC4gQS48L2F1dGhvcj48YXV0aG9yPIBIYXJzb24sIEou IFYuPC9h dXRob3I+PGF1dGhvcj5NY1BoZXJzb24sIEouIEQuPC9hdXRob3I+PGF1dGhvcj5HaW JicywgUi4g QS48L2F1dGhvcj48YXV0aG9yPkdyaW1tb25kLCBTLiBNLjwvYXV0aG9yPjwvYXV0 aG9ycz48L2Nv bnRyaWJ1dG9ycz48YXVOaC1hZGRyZXNzPlRoZSBLaW5naG9ybiBDYW5jZXIgQ2 VudHJ1LCAzNzAg VmljdG9yaWEgU3RyZWVOLCBEYXJsaW5naHVyc3QsIFN5ZG51eSwgTmV3IFNvdX RoIFdhbGVzIDIw MTAsIEF 1c3RyYWxpYS48L2F1dGgtYWRkcmVzcz48dGl0bGVzPjx0aXRsZT5QYW5 jcmVhdGljIGNh bmNlciBnZW5vbWVzIHJldmVhbCBhYmVycmFOaW9ucyBpbiBheG9uIGd 1aWRhbm N1IHBhdGh3YXkg Z2VuZXM8L3RpdGxlPjxzZWNvbmRhcnktdGl0bGU+TmF0dXJlPC9zZWNvbmRhcnk tdGl0bGU+PGFs dC1OaXRsZT5OYXR1cmU8L2FsdC1OaXRsZT48L3RpdGxlcz48cGVyaW9kaWNhbD4 8ZnVsbC1OaXRs ZT5OYXR1cmU8L2ZlbGwtdGl0bGU+PGFiYnItMT5OYXR1cmU8L2FiYnItMT48L3 BlcmlvZGljYWw+PGFsdC1wZXJpb2RpY2FsPjxmdWxsLXRpdGxlPk5hdHVyZTwvZnVsbC1OaXRsZT48 YWJici0xPk5h dHVyZTwvYWJiciOxPjwvYWx0LXBlcmlvZGljYWw+PHBhZ2VzPjM50S00MDU8L3 BhZ2VzPjx2b2x1 bWU+NDkxPC92b2x1bWU+PG51bWJlcj43NDIOPC9udW liZXI+PGtleXdvcmRzPjxrZ X13b3JkPkFu aW1hbHM8L2tleXdvcmQ+PGtleXdvcmQ+QXhvbnMvKm11dGFib2xpc208L2tleXdvc mQ+PGtleXdv cmQ+Q2FyY2lub21hLCBQYW5jcmVhdGljIER1Y3RhbC8qZ2VuZXRpY3MvKnBhdG hvbG9neTwva2V5 d29yZD48a2V5d29yZD5HZW51IERvc2FnZTwva2V5d29yZD48a2V5d29yZD5HZW51I EV4cHJ1c3Np b24gUmVndWxhdGlvbiwgTmVvcGxhc3RpYzwva2V5d29yZD48a2V5d29yZD5HZW5 vbWUvKmdlbmVO aWNzPC9rZX13b3JkPjxrZX13b3JkPkh1bWFuczwva2V5d29yZD48a2V5d29yZD5LYX BsYW4tTWVp ZXIgRXNOaW1hdGU8L2tleXdvcmQ+PGtleXdvcmQ+TWljZTwva2V5d29yZD48a2V5 d29yZD5NdXRh dGlvbjwva2V5d29yZD48a2V5d29yZD5QYW5jcmVhdGljIE51b3BsYXNtcy8qZ2VuZX RpY3MvKnBh dGhvbG9neTwva2V5d29yZD48a2V5d29yZD5Qcm90ZWlucy9nZW5ldGljczwva2V5d2 9yZD48a2V5 d29yZD5TaWduYWwgVHJhbnNkdWNOaW9uPC9rZX13b3JkPjwva2V5d29yZHM+PG RhdGVzPjx5ZWFy PjIwMTI8L311YXI+PHB1YilkYXR1cz48ZGF0ZT50b3YgMTU8L2RhdGU+PC9wdWI tZGF0ZXM+PC9k YXRlcz48aXNibj4xNDc2LTQ2ODcgKEVsZWN0cm9uaWMpJiN4RDswMDI4LTA4M zYgKExpbmtpbmcp PC9pc2JuPjxhY2Nlc3Npb24tbnVtPjIzMTAzODY5PC9hY2N1c3Npb24tbnVtPjx1cmxzP jxyZWxh dGVkLXVybHM+PHVybD5odHRwOi8vd3d3Lm5jYmkubmxtLm5paC5nb3YvcHVib WVkLzIzMTAzODY5 PC91cmw+PC9yZWxhdGVkLXVybHM+PC91cmxzPjxjdXN0b20yPjM1MzA4OTg8L2 Nic3RvbTI+PGVs ZWN0cm9uaWMtcmVzb3VyY2UtbnVtPjEwLjEwMzgvbmF0dXJ1MTE1NDc8L2VsZ WN0cm9uaWMtcmVz b3VyY2UtbnVtPjwvcmVjb3JkPjwvQ210ZT48L0VuZE5vdGU+PEVuZE5vdGU+PENpdGU+PEF1dGhvcj5CaWFua21uPC9BdXRob3I+PFIIYXI+MjAx MjwvWWVhcj48 UmVj TnVtPjI8L1J1Y051bT48RG1zcGxheVRleHQ+PHNOeWx1IGZhY2U9InN1cGVyc2 NyaXBOIj4y PC9zdHlsZT48L0Rpc3BsYX1UZXhOPjxyZWNvcmQ+PHJ1Yy 1udW1iZXI+MjwvcmVj LW5lbWJlcj48 Zm9yZWlnbi1rZXlzPjxrZXkgYXBwPSJFTiIgZGItaWQ9IndkdmQyeDJ3NDJkOXgzZX h4YW54NWRw ZXJkZWRwdHRhMnhmeCI+Mjwva2V5PjwvZm9yZWlnbi1rZXlzPjxyZWYtdHlwZSB uYW11PSJKb3Vy bmFsIEFydGljbGUiPjE3PC9yZWYtdH1wZT48Y29udHJpYnVOb3JzPjxhdXRob3JzPjxh dXRob3I+QmlhbmtpbiwgQS4gVi48L2F1dGhvcj48YXV0aG9yPldhZGRlbGwsIE4uPC9hdXRob3I+PGF1 dGhv cj5LYXNzYWhuLCBLLiBTLjwvYXV0aG9yPjxhdXRob3I+R2luZ3JhcywgTS4gQy48 L2F1dGhvcj48 YXV0aG9yPk11dGh1c3dhbXksIEwuIEIuPC9hdXRob3I+PGF1dGhvcj5Kb2hucywgQ S 4gTC48L2F1 dGhvcj48YXV0aG9yPk1pbGxlciwgRC4gSy48L2F1dGhvcj48YXV0aG9yPldpbHNvbiw gUC4gSi48 L2F1dGhvcj48YXV0aG9yPlBhdGNoLCBBLiBNLjwvYXV0aG9yPjxhdXRob3I+V3Us IEouPC9hdXRo b3I+PGF1dGhvcj5DaGFuZywgRC4gSy48L2F1dGhvcj48YXV0aG9yPkNvd2xleSwgTS 4gSi48L2F1 dGhvcj48YXV0aG9yPkdhcmRpbmVyLCBCLiBCLjwvYXV0aG9yPjxhdXRob3I+U29u ZywgUy48L2F1 dGhvcj48YXV0aG9yPkhhcmxpd29uZywgSS48L2F1dGhvcj48YXV0aG9yPklkcmlzb2d sdSwgUy48 L2F1dGhvcj48YXV0aG9yPk5vdXJzZSwgQy48L2F1dGhvcj48YXV0aG9yPk5vdXJiY Wtoc2gsIEUu PC9hdXRob3I+PGF1dGhvcj5NYW5uaW5nLCBTLjwvYXV0aG9yPjxhdXRob3I+V2Fu aSwgUy48L2F1 dGhvcj48YXV0aG9yPkdvbmdvcmEsIEOuPC9hdXRob3I+PGF1dGhvcj5QYWppYywg T S48L2F1dGhv cj48YXV0aG9yP1NjYXJsZXROLCBDLiBKLjwvYXV0aG9yPjxhdXRob3I+R21sbCwg QS4gSi48L2F1 dGhvcj48YXV0aG9yPlBpbmhvLCBBLiBWLjwvYXV0aG9yPjxhdXRob3I+Um9vbWF uLCBJLjwvYXVO aG9yPjxhdXRob3I+QW5kZXJzb24sIEOuPC9hdXRob3I+PGF1dGhvcj5Ib2xtZXMsIE8u PC9hdXRo b3I+PGF1dGhvcj5MZW9uYXJkLCBDLjwvYXV0aG9yPjxhdXRob3I+VGF5bG9yLCB ELjwvYXV0aG9y PjxhdXRob3I+V29vZCwgUy48L2F1dGhvcj48YXV0aG9yPlh1LCBRLjwvYXV0aG9yP jxhdXRob3I+Tm9uZXMsIEsuPC9hdXRob3I+PGF1dGhvcj5GaW5rLCBKLiBMLjwvYXV0aG9yPjxh dXRob3I+Q2 hy aXNOLCBBLjwvYXV0aG9yPjxhdXRob3I+QnJ1eG5lciwgVC48L2F1dGhvcj48YXVOa G9yPkNsb29u YW4sIE4uPC9hdXRob3I+PGF1dGhvcj5Lb2xsZSwgRy48L2F1dGhvcj48YXV0aG9yPk 5ld2VsbCwg Ri48L2F1dGhvcj48YXV0aG9yPlBpbmVzZSwgT S48L2F1dGhvcj48YXV0aG9yPk11Y WQsIFIuIFMu PC9hdXRob3I+PGF1dGhvcj5IdW1waHJpcywgSi4gTC48L2F1dGhvcj48YXV0aG9yPkt hcGxhbiwg Vy48L2F1dGhvcj48YXV0aG9yPkpvbmVzLCBNLiBELjwvYXV0aG9yPjxhdXRob3I+Q29sdm1uLCBF LiBLLjwvYXV0aG9yPjxhdXRob3I+TmFncmlhbCwgQS4gTS48L2F1dGhvcj48YXVOa G9yPkhlbXBo cmV5LCBFLiBTLjwvYXV0aG9yPjxhdXRob3I+Q2hvdSwgQS48L2F1dGhvcj48YXV0 aG9yPkNoaW4s IFYuIFQuPC9hdXRob3I+PGF1dGhvcj5DaGFudHJpbGwsIEwuIEEuPC9hdXRob3I+PG F1dGhvcj5N YXdzb24sIEEuPC9hdXRob3I+PGF1dGhvcj5TYW1yYSwgSi4gUy48L2F1dGhvcj48Y XV0aG9yPktl bmNoLCBKLiBHLjwvYXV0aG9yPjxhdXRob3I+TG92ZWxsLCBKLiBBLjwvYXVOa G9yPjxhdXRob3I+RGFseSwgUi4gSi48L2F1dGhvcj48YXV0aG9yPk11cnJldHQsIE4uIEQuPC9hdXRob3I+PGF1 dGhv cj5Ub29uLCBDLjwvYXV0aG9yPjxhdXRob3I+RXBhcmksIEsuPC9hdXRob3I+PGF1d Ghvcj50Z3V5 ZW4sIE4uIFEuPC9hdXRob3I+PGF1dGhvcj5CYXJib3VyLCBBLjwvYXV0aG9yPjxhd XRob3I+WmVw cywgTi48L2F1dGhvcj48YXV0aG9yPkF 1c3RyYWxpYW4gUGFuY3J1YXRpYyBDYW 5jZXIgR2Vub211 LCBJbm10aWFOaXZ1PC9hdXRob3I+PGF1dGhvcj5LYWtrYXIsIE4uPC9hdXRob3I+PG F1dGhvcj5a aGFvLCBGLjwvYXV0aG9yPjxhdXRob3I+V3UsIFkuIFEuPC9hdXRob3I+PGF1dGhvcj 5XYW5nLCBN LjwvYXV0aG9yPjxhdXRob3I+TXV6bnksIEQuIEOuPC9hdXRob3I+PGF1dGhvcj5GaX NoZXIsIFcu IEUuPC9hdXRob3I+PGF1dGhvcj5CenVuaWNhcmRpLCBGLiBDLjwvYXV0aG9yPjx hdXRob3I+SG9k Z2VzLCBTLiBFLjwvYXV0aG9yPjxhdXRob3I+UmVpZCwgSi4gRy48L2F1dGhvcj48 YXV0aG9yPkRy dW1tb25kLCBKLjwvYXV0aG9yPjxhdXRob3I+Q2hhbmcslEsuPC9hdXRob3I+PGF1d Ghvcj5IYW4s IFkuPC9hdXRob3I+PGF1dGhvcj5MZXdpcywgTC4gUi48L2F1dGhvcj48YXV0aG9yPk RpbmgsIEgu PC9hdXRob3I+PGF1dGhvcj5CdWhheSwgQy4gSi48L2F1dGhvcj48YXV0aG9yPkJlY2s sIFQuPC9h dXRob3I+PGF1dGhvcj5UaW1tcywgTC48L2F1dGhvcj48YXV0aG9yPlNhbSwgTS48L2 F1dGhvcj48 YXV0aG9yPkJ1Z2xleSwgSy48L2F1dGhvcj48YXV0aG9yPkJyb3duLCBBLjwvYXVOa G9yPjxhdXRo b3I+UGFpLCBELjwvYXV0aG9yPjxhdXRob3I+UGFuY2hhbCwgQS48L2F1dGhvcj48 YXV0aG9yPkJ1 Y2huZXIsIE4uPC9hdXRob3I+PGF1dGhvcj5EZSBCb3JqYSwgUi48L2F1dGhvcj48YX V0aG9yPkR1 bnJvY2hlLCBSLiBFLjwvYXV0aG9yPjxhdXRob3I+WXVuZywgQy4gSy48L2F1dGhvc j48YXV0aG9y P1N1cnJhLCBTLjwvYXV0aG9yPjxhdXRob3I+T251dHRvLCBOLjwvYXV0aG9yPjxhd XRob3I+TXVr aG9wYWRoeWF5LCBELjwvYXV0aG9yPjxhdXRob3I+VHNhbywgT S4gUy48L2F1dG hvcj48YXV0aG9y P1NoYXcsIFAuIEEuPC9hdXRob3I+PGF1dGhvcj5QZXRlcnNlbiwgRy4gTS48L2F1dGh vcj48YXV0 aG9yPkdhbGxpbmdlciwgUy48L2F1dGhvcj48YXV0aG9yPkhydWJhbiwgUi4gSC48L2F1 dGhvcj48 YXV0aG9yPk1haXRyYSwgQS48L2F1dGhvcj48YXV0aG9yPklhY29idXppby1Eb25 ha HVILCBDLiBB LjwvYXV0aG9yPjxhdXRob3I+U2NodWxpY2ssIFIuIEQuPC9hdXRob3I+PGF1dGhvcj 5Xb2xmZ2Fu ZywgQy4gTC48L2F1dGhvcj48YXV0aG9yPk1vcmdhbiwgUi4gQS48L2F1dGhvcj48YX V0aG9yPkxh d2xvciwgUi4gVC48L2F1dGhvcj48YXV0aG9yPkNhcGVsbGksIFAuPC9hdXRob3I+PG F1dGhvcj5D b3JibywgVi48L2F1dGhvcj48YXV0aG9yP1NjYXJkb25pLCBNLjwvYXV0aG9yPjxhdX Rob3I+VG9y dG9yYSwgRy48L2F1dGhvcj48YXV0aG9yPlRlbXBlcm8sIEOuIEEuPC9hdXRob3I+PG F1dGhvcj5N YW5uLCBLLiBNLjwvYXV0aG9yPjxhdXRob3I+SmVua2lucywgTi4gQS48L2F1dGhv cj48YXV0aG9y P1B1cmV6LU1hbmNlcmEsIFAuIEEuPC9hdXRob3I+PGF1dGhvcj5BZGFtcywgRC4gSi 48L2F1dGhv cj48YXV0aG9yPkxhcmdhZXNwYWRhLCBELiBBLjwvYXV0aG9yPjxhdXRob3I+V2 Vzc2VscywgTC4g Ri48L2F1dGhvcj48YXV0aG9yP1J1c3QsIEEuIEcuPC9hdXRob3I+PGF1dGhvcj5TdGVp biwgTC4g RC48L2F1dGhvcj48YXV0aG9yP1R1dmVzb24sIEQuIEEuPC9hdXRob3I+PGF1dGhvcj 5Db3B1bGFu ZCwgTi4gRy48L2F1dGhvcj48YXV0aG9yPk11c2dyb3Z1LCBFLiBBLjwvYXV0aG9yPj xhdXRob3I+U2NhcnBhLCBBLjwvYXV0aG9yPjxhdXRob3I+RXNobGVtYW4sIEouIFIuPC9hdXRo b3I+PGF1dGhv cj5IdWRzb24sIFQuIEouPC9hdXRob3I+PGF1dGhvcj5TdXRoZXJsYW5kLCBSLiBMLj wvYXV0aG9y PjxhdXRob3I+V2hlZWxlciwgRC4gQS48L2F1dGhvcj48YXV0aG9yP1B1YXJzb24sIEou IFYuPC9h dXRob3I+PGF1dGhvcj5NY1BoZXJzb24sIEouIEQuPC9hdXRob3I+PGF1dGhvcj5HaW JicywgUi4g QS48L2F1dGhvcj48YXV0aG9yPkdyaW1tb25kLCBTLiBNLjwvYXV0aG9yPjwvYXV0 aG9ycz48L2Nv bnRyaWJ1dG9ycz48YXVOaC1hZGRyZXNzP1RoZSBLaW5naG9ybiBDYW5jZXIgQ2 VudHJ1LCAzNzAg VmljdG9yaWEgU3RyZWVOLCBEYXJsaW5naHVyc3QsIFN5ZG51eSwgTmV3IFNvdX RoIFdhbGVzIDIw MTAsIEF 1c3RyYWxpYS48L2F1dGgtYWRkcmVzcz48dGl0bGVzPjx0aXRsZT5QYW5 jcmVhdGljIGNh bmNlciBnZW5vbWVzIHJldmVhbCBhYmVycmFOaW9ucyBpbiBheG9uIGd 1aWRhbm N1IHBhdGh3YXkg Z2VuZXM8L3RpdGxlPjxzZWNvbmRhcnktdGl0bGU+TmF0dXJlPC9zZWNvbmRhcnk tdGl0bGU+PGFs dC1OaXRsZT5OYXR1cmU8L2FsdC1OaXRsZT48L3RpdGxlcz48cGVyaW9kaWNhbD4 8ZnVsbC1OaXRs ZT5OYXR1cmU8L2ZlbGwtdGl0bGU+PGFiYnItMT5OYXR1cmU8L2FiYnItMT48L3 BlcmlvZGljYWw+PGFsdC1wZXJpb2RpY2FsPjxmdWxsLXRpdGxlPk5hdHVyZTwvZnVsbC1OaXRsZT48 YWJici0xPk5h dHVyZTwvYWJiciOxPjwvYWx0LXBlcmlvZGljYWw+PHBhZ2VzPjM50S00MDU8L3 BhZ2VzPjx2b2x1 bWU+NDkxPC92b2x1bWU+PG51bWJlcj43NDIOPC9udW liZXI+PGtleXdvcmRzPjxrZ X13b3JkPkFu aW1hbHM8L2tleXdvcmQ+PGtleXdvcmQ+QXhvbnMvKm11dGFib2xpc208L2tleXdvc mQ+PGtleXdv cmQ+Q2FyY2lub21hLCBQYW5jcmVhdGljIER1Y3RhbC8qZ2VuZXRpY3MvKnBhdG hvbG9neTwva2V5 d29yZD48a2V5d29yZD5HZW51IERvc2FnZTwva2V5d29yZD48a2V5d29yZD5HZW51I EV4cHJ1c3Np b24gUmVndWxhdGlvbiwgTmVvcGxhc3RpYzwva2V5d29yZD48a2V5d29yZD5HZW5 vbWUvKmdlbmVO aWNzPC9rZX13b3JkPjxrZX13b3JkPkh1bWFuczwva2V5d29yZD48a2V5d29yZD5LYX BsYW4tTWVp ZXIgRXNOaW1hdGU8L2tleXdvcmQ+PGtleXdvcmQ+TWljZTwva2V5d29yZD48a2V5 d29yZD5NdXRh dGlvbjwva2V5d29yZD48a2V5d29yZD5QYW5jcmVhdGljIE5lb3BsYXNtcy8qZ2VuZX RpY3MvKnBh dGhvbG9neTwva2V5d29yZD48a2V5d29yZD5Qcm90ZWlucy9nZW5ldGljczwva2V5d2 9yZD48a2V5 d29yZD5TaWduYWwgVHJhbnNkdWNOaW9uPC9rZX13b3JkPjwva2V5d29yZHM+PG RhdGVzPjx5ZWFy PjIwMTI8L311YXI+PHB1YilkYXRlcz48ZGF0ZT50b3YgMTU8L2RhdGU+PC9wdWI tZGF0ZXM+PC9k YXRlcz48aXNibj4xNDc2LTQ2ODcgKEVsZWN0cm9uaWMpJiN4RDswMDI4LTA4M zYgKExpbmtpbmcp PC9pc2JuPjxhY2Nlc3Npb24tbnVtPjIzMTAzODY5PC9hY2Nlc3Npb24tbnVtPjx1cmxzP jxyZWxh dGVkLXVybHM+PHVybD5odHRwOi8vd3d3Lm5jYmkubmxtLm5paC5nb3YvcHVib WVkLzIzMTAzODY5 PC91cmw+PC9yZWxhdGVkLXVybHM+PC91cmxzPjxjdXN0b20yPjM1MzA4OTg8L2 Nlc3RvbTI+PGVs ZWN0cm9uaWMtcmVzb3VyY2UtbnVtPjEwLjEwMzgvbmF0dXJlMTE1NDc8L2VsZ WN0cm9uaWMtcmVz b3VyY2UtbnVtPjwvcmVjb3JkPjwvQ210ZT48L0VuZE5vdGU+2, we enriched for neoplastic cells either by macrodissecting primary tumors or by flow-sorting tumor nuclei (example 1), and performed high-coverage sequencing of these enriched samples. We obtained a per-base sequencing coverage of 234-fold for each tumor analyzed by whole-exome sequencing and 754-fold for each tumor analyzed by targeted cancer gene sequencing (Example 1).

We detected an average of 114 tumor-specific (somatic) non-synonymous sequence alterations in the cancers analyzed by whole exome sequencing, similar to previous studies of this tumor type2,3, and 4.7 non-synonymous sequence alterations per cancer in the targeted analyses (Supplementary Table 4). Among known recurrent sequence alterations in the cancers analyzed we identified mutations in the known pancreatic cancer driver genes: KRAS (88%), TP53 (77%), SMAD4 (29%), CDKN2A (18%), and TGFBR2 (7%) (Supplementary Tables 4 and 5)2,3. Homozygous deletions were difficult to assess given the low purity of the samples, but such alterations were identified in CDKN2A in an additional 5% of cases.

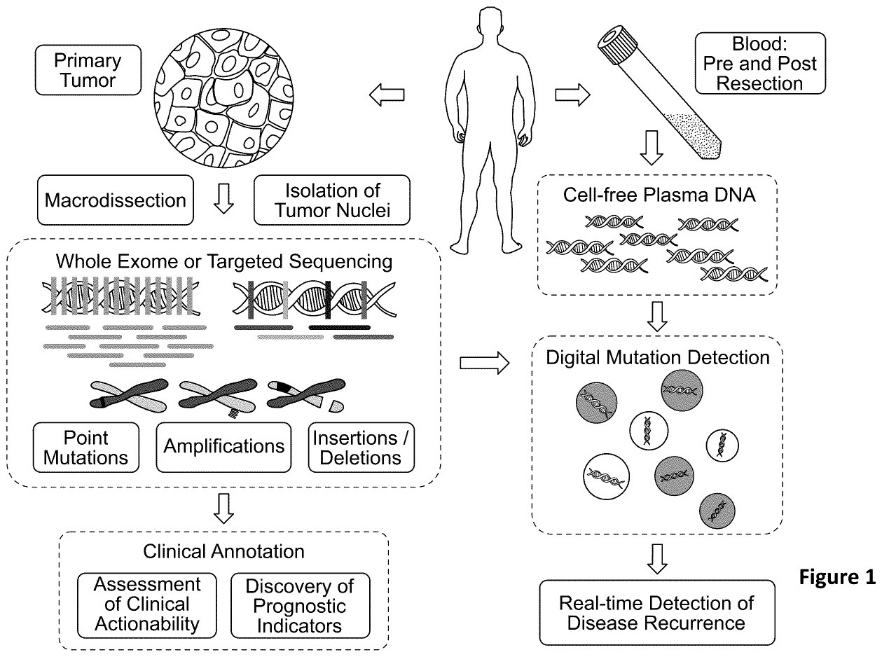

We also identified recurrent somatic alterations in genes involved in chromatin regulation or modification, primarily involving the AT-rich interactive domain-containing ARIDIA gene (9% of cases) and the histone methyltransferase MLL3 gene (7%) (Supplementary FIG. 1, Supplementary Tables 4 and 5)PEVuZE5vdGU+PENpdGU+PEF1dGhvcj5Kb251czwvQXV0aG9yPjxZZWFyPjIwMT I8L111YXI+PFJl Y051bT4yNDwvUmVjTnVtPjxEaXNwbGF5VGV4dD48c3R5bGUgZmFjZT0ic3VwZX JzY3JpcHQiPjY8 L3N0eWxlPjwvRGlzcGxheVRleHQ+PHJlY29yZD48cmVjLW51bWJlcj4yNDwvcmVj LW5lbWJlcj48 Zm9yZWlnbi1rZXlzPjxrZXkgYXBwPSJFTiIgZGItaWQ9IndkdmQyeDJ3NDJkOXgzZX h4YW54NWRw ZXJkZWRwdHRhMnhmeCI+MjQ8L2tleT48L2ZvcmVpZ24ta2V5cz48cmVmLXR5cG UgbmFtZT0iSm91 cm5hbCBBcnRpY2xlIj4xNzwvcmVmLXR5cGU+PGNvbnRyaWJ1dG9ycz48YXV0aG9 ycz48YXV0aG9y PkpvbmVzLCBTLjwvYXV0aG9yPjxhdXRob3I+TGksIEOuPC9hdXRob3I+PGF1dGhvc j5QYXJzb25z LCBELiBXLjwvYXV0aG9yPjxhdXRob3I+WmhhbmcsIFguPC9hdXRob3I+PGF1dGhv cj5XZXNzZWxp bmcsIEouPC9hdXRob3I+PGF1dGhvcj5LcmlzdGVsLCBQLjwvYXV0aG9yPjxhdXRob 3I+U2NobWlk dCwgTS4gSy48L2F1dGhvcj48YXV0aG9yPk1hcmtvd21OeiwgUy48L2F1dGhvcj48YXV 0aG9yPllh biwgSC48L2F1dGhvcj48YXV0aG9yPkJpZ251ciwgRC48L2F1dGhvcj48YXV0aG9yPkh ydWJhbiwg Ui4gSC48L2F1dGhvcj48YXV0aG9yPkVzaGxlbWFuLCBKLiBSLjwvYXV0aG9yPjxhd XRob3I+SWFj b2J1emlvLURvbmFodWUsIEMuIEEuPC9hdXRob3I+PGF1dGhvcj5Hb2dnaW5zLCBN LjwvYXV0aG9y PjxhdXRob3I+TWFpdHJhLCBBLjwvYXV0aG9yPjxhdXRob3I+TWFsZWssIFMuIE4u PC9hdXRob3I+PGF1dGhvcj5Qb3dlbGwsIFMuPC9hdXRob3I+PGF1dGhvcj5Wb2dlbHNOZW1uLCBCL jwvYXV0aG9y PjxhdXRob3I+S21uemxlciwgSy4gVy48L2F1dGhvcj48YXV0aG9yP1Z1bGN1bGVzY3U sIFYuIEUu PC9hdXRob3I+PGF1dGhvcj5QYXBhZG9wb3Vsb3MsIE4uPC9hdXRob3I+PC9hdXRo b3JzPjwvY29u dHJpYnVOb3JzPjxhdXRoLWFkZHJ1c3M+THVkd2lnIENlbnRlciBmb3IgQ2FuY2VyIE dlbmV0aWNz IGFuZCBUaGVyYXB1dXRpY3MgYW5kIEhvd2FyZCBIdWdoZXMgTWVkaWNhbCB JbnN0aXR1 dGUsIEpv aG5zIEhvcGtpbnMgS21tbWVsIENhbmNlciBDZW5OZXIsIEJhbHRpbW9yZ SwgTWFye WxhbmQsIFVT QS48L2F1dGgtYWRkcmVzcz48dGl0bGVzPjx0aXRsZT5Tb21hdGljIG11dGF0aW9ucy BpbiB0aGUg Y2hyb21hdGluIHJlbW9kZWxpbmcgZ2VuZ SBBUklEMUEgb2NjdXIgaW4gc2V2ZXJh bCBOdW lvciB0 eXBlczwvdGl0bGU+PHN1Y29uZGFyeSOaXRsZT5IdWOgTXVOYXQ8L3N1Y29uZGF yeS10aXRsZT48 YWx0LXRpdGxlPkhlbWFuIG11dGF0aW9uPC9hbHQtdGl0bGU+PC90aXRsZXM+PH BlcmlvZGljYWw+PGZ1bGwtdGl0bGU+SHVtIE11dGFOPC9mdWxsLXRpdGxlPjxhYmJyLTE+SHVtYW 4gbXV0YXRpb248 L2FiYnItMT48L3B1cmlvZGljYWw+PGFsdC1wZXJpb2RpY2FsPjxmdWxsLXRpdGxlP khlbSBNdXRh dDwvZnVsbC1OaXRsZT48YWJiciOxPkh1bWFuIG1 1dGF0aW9uPC9hYmJyLTE+PC9h bHQtcGVyaW9k aWNhbD48cGFnZXM+MTAwLTM8L3BhZ2VzPjx2b2x1bWU+MzM8L3ZvbHVtZT48 bnVtYmVyPjE8L251 bWJlcj48a2V5d29yZHM+PGtleXdvcmQ+QWRlbm9jYXJjaW5vbWEsIENsZWFyIEN1 bGwvKmdlbmVO aWNzL3BhdGhvbG9neTwva2V5d29yZD48a2V5d29yZD5CYXNIIFNIcXVIbmNIPC9r ZX13b3JkPjxr ZX13b3JkPkJyZWFzdC9tZXRhYm9saXNtL3BhdGhvbG9neTwva2V5d29yZD48a2V5d 29yZD5DaHJv bWFOaW4vKmdlbmV0aWNzL21ldGFib2xpc2O8L2tleXdvcmQ+PGtleXdvcmQ+Q2hyb 21hdGluIEFz c2VtYmx5IGFuZCBEaXNhc3NlbWJseTwva2V5d29yZD48a2V5d29yZD5Db2xvbi9tZ XRhYm9saXNt L3BhdGhvbG9neTwva2V5d29yZD48a2V5d29yZD5Db2xvcmVjdGFsIE51b3BsYXNtcy 8qZ2VuZXRp Y3MvcGF0aG9sb2d5PC9rZX13b3JkPjxrZX13b3JkPkZlbWFsZTwva2V5d29yZD48a2V 5d29yZD4q RnJhbWVzaGlmdCBNdXRhdGlvbjwva2V5d29yZD48a2V5d29yZD5IdW1hbnM8L2tle XdvcmQ+PGtl eXdvcmQ+TWFsZTwva2V5d29yZD48a2V5d29yZD5NaWNyb3NhdGVsbG1OZSBJbnN OYWJpbG1OeTwv a2V5d29yZD48a2V5d29yZD5NaWNyb3NhdGVsbGl0ZSBSZXB1YXRzPC9rZX13b3Jk PjxrZX13b3Jk Pk1vbGVjdWxhciBTZXF1ZW5jZSBEYXRhPC9rZX13b3JkPjxrZX13b3JkPk51Y2x1YX IgUHJvdGVp bnMvKmdlbmV0aWNzPC9rZX13b3JkPjxrZX13b3 JkPlBhbmNyZWFzL21ldGFib2xpc2O vcGF0aG9s b2d5PC9rZX13b3JkPjxrZX13b3JkP1N0b21hY2gvbWVOYWJvbGlzbS9wYXRob2xvZ3k 8L2tleXdv cmQ+PGtleXdvcmQ+U3RvbWFjaCBOZW9wbGFzbXMvKmdlbmV0aWNzL3BhdGhv bG9neTwva2V5d29y ZD48a2V5d29yZD5UcmFuc2NyaXBOaW9uIEZhY3RvcnMvKmdlbmV0aWNzPC9rZXl 3b3JkPjwva2V5 d29yZHM+PGRhdGVzPjx5ZWFyPjIwMTI8L311YXI+PHB1Yi1kYXR1cz48ZGF0ZT5K YW48L2RhdGU+PC9wdWItZGF0ZXM+PC9kYXRlcz48aXNibj4xMDk4LTEwMDQgKEVsZWN0cm9u aWMpJiN4RDsxMDU5 LTc3OTQgKExpbmtpbmcpPC9pc2JuPjxhY2Nlc3Npb24tbnVtPjIyMDA5OTQxPC9hY2 Nlc3Npb24t bnVtPjxlcmxzPjxyZWxhdGVkLXVybHM+PHVybD5odHRwOi8vd3d3Lm5jYmkubmx tLm5paC5nb3Yv cHVibWVkLzIyMDA50TQxPC91cmw+PC9yZWxhdGVkLXVybHM+PC91cmxzPjxjd XN0b20yPjMyNDA3 MTk8L2N1c3RvbTI+PGVsZWN0cm9uaWMtcmVzb3VyY2UtbnVtPjEwLjEwMDIvaH VtdS4yMTYzMzwv ZWxlY3Ryb25pYy1yZXNvdXJjZSiudWO+PC9yZWNvcmQ+PC9DaXR1PjwvRW5kT m90ZT4A PEVuZE5vdGU+PENpdGU+PEF1dGhvcj5Kb251czwvQXV0aG9yPjxZZWFyPjIwMTI8 LI11YXI+PFJ1 Y051bT4yNDwvUmVjTnVtPjxEaXNwbGF5VGV4dD48c3R5bGUgZmFjZT0ic3VwZX JzY3JpcHQiPjY8 L3NOeWx1PjwvRGlzcGxheVRleHQ+PHJ1Y29yZD48cmVjLW51bWJlcj4yNDwvcmVj LW51bWJlcj48 Zm9yZWlnbi1rZXlzPjxrZXkgYXBwPSJFTiIgZGItaWQ9IndkdmQyeDJ3NDJkOXgzZX h4YW54NWRw ZXJkZWRwdHRhMnhmeCI+MjQ8L2tleT48L2ZvcmVpZ24ta2V5cz48cmVmLXR5cG UgbmFtZT0iSm91 cm5hbCBBcnRpY2xlIj4xNzwvcmVmLXR5cGU+PGNvbnRyaWJ1dG9ycz48YXV0aG9 ycz48YXV0aG9y PkpvbmVzLCBTLjwvYXV0aG9yPjxhdXRob3I+TGksIEOuPC9hdXRob3I+PGF1dGhvc j5QYXJzb25z LCBELiBXLjwvYXV0aG9yPjxhdXRob3I+WmhhbmcsIFguPC9hdXRob3I+PGF1dGhv cj5XZXNzZWxp bmcsIEouPC9hdXRob3I+PGF1dGhvcj5LcmlzdGVsLCBQLjwvYXV0aG9yPjxhdXRob 3I+U2NobWlk dCwgTS4gSy48L2F1dGhvcj48YXV0aG9yPk1hcmtvd21OeiwgUy48L2F1dGhvcj48YXV 0aG9yPllh biwgSC48L2F1dGhvcj48YXV0aG9yPkJpZ251ciwgRC48L2F1dGhvcj48YXV0aG9yPkh ydWJhbiwg Ui4gSC48L2F1dGhvcj48YXV0aG9yPkVzaGxlbWFuLCBKLiBSLjwvYXV0aG9yPjxhd XRob3I+SWFj b2J1emlvLURvbmFodWUsIEMuIEEuPC9hdXRob3I+PGF1dGhvcj5Hb2dnaW5zLCBN LjwvYXV0aG9y PjxhdXRob3I+TWFpdHJhLCBBLjwvYXV0aG9yPjxhdXRob3I+TWFsZWssIFMuIE4u PC9hdXRob3I+PGF1dGhvcj5Qb3dlbGwsIFMuPC9hdXRob3I+PGF1dGhvcj5Wb2dlbHNOZW1uLCBCL jwvYXV0aG9y PjxhdXRob3I+S21uemxlciwgSy4gVy48L2F1dGhvcj48YXV0aG9yP1Z1bGN1bGVzY3U sIFYuIEUu PC9hdXRob3I+PGF1dGhvcj5QYXBhZG9wb3Vsb3MsIE4uPC9hdXRob3I+PC9hdXRo b3JzPjwvY29u dHJpYnVOb3JzPjxhdXRoLWFkZHJ1c3M+THVkd2lnIENlbnRlciBmb3IgQ2FuY2VyIE dlbmV0aWNz IGFuZCBUaGVyYXB1dXRpY3MgYW5kIEhvd2FyZCBIdWdoZXMgTWVkaWNhbCB JbnN0aXR1 dGUsIEpv aG5zIEhvcGtpbnMgS21tbWVsIENhbmNlciBDZW5OZXIsIEJhbHRpbW9yZ SwgTWFye WxhbmQsIFVT QS48L2F1dGgtYWRkcmVzcz48dGl0bGVzPjx0aXRsZT5Tb21hdGljIG11dGF0aW9ucy BpbiB0aGUg Y2hyb21hdGluIHJlbW9kZWxpbmcgZ2VuZ SBBUklEMUEgb2NjdXIgaW4gc2V2ZXJh bCBOdW lvciBO eXBlczwvdGl0bGU+PHN1Y29uZGFyeSOaXRsZT5IdWOgTXVOYXQ8L3N1Y29uZGF yeS1OaXRsZT48 YWx0LXRpdGxlPkhlbWFuIG11dGF0aW9uPC9hbHQtdGl0bGU+PC90aXRsZXM+PH BlcmlvZGljYWw+PGZ1bGwtdGl0bGU+SHVtIE11dGFOPC9mdWxsLXRpdGxlPjxhYmJyLTE+SHVtYW 4gbXV0YXRpb248 L2FiYnItMT48L3B1cmlvZGljYWw+PGFsdC1wZXJpb2RpY2FsPjxmdWxsLXRpdGxlP khlbSBNdXRh dDwvZnVsbC1OaXRsZT48YWJiciOxPkh1bWFuIG1 1dGF0aW9uPC9hYmJyLTE+PC9h bHQtcGVyaW9k aWNhbD48cGFnZXM+MTAwLTM8L3BhZ2VzPjx2b2x1bWU+MzM8L3ZvbHVtZT48 bnVtYmVyPjE8L251 bWJlcj48a2V5d29yZHM+PGtleXdvcmQ+QWRlbm9jYXJjaW5vbWEsIENsZWFyIEN1 bGwvKmdlbmVO aWNzL3BhdGhvbG9neTwva2V5d29yZD48a2V5d29yZD5CYXNIIFNIcXVIbmNIPC9r ZX13b3JkPjxr ZX13b3JkPkJyZWFzdC9tZXRhYm9saXNtL3BhdGhvbG9neTwva2V5d29yZD48a2V5d 29yZD5DaHJv bWFOaW4vKmdlbmV0aWNzL21ldGFib2xpc2O8L2tleXdvcmQ+PGtleXdvcmQ+Q2hyb 21hdGluIEFz c2VtYmx5IGFuZCBEaXNhc3NlbWJseTwva2V5d29yZD48a2V5d29yZD5Db2xvbi9tZ XRhYm9saXNt L3BhdGhvbG9neTwva2V5d29yZD48a2V5d29yZD5Db2xvcmVjdGFsIE51b3BsYXNtcy 8qZ2VuZXRp Y3MvcGF0aG9sb2d5PC9rZX13b3JkPjxrZX13b3JkPkZlbWFsZTwva2V5d29yZD48a2V 5d29yZD4q RnJhbWVzaGlmdCBNdXRhdGlvbjwva2V5d29yZD48a2V5d29yZD5IdW1hbnM8L2tle XdvcmQ+PGtl eXdvcmQ+TWFsZTwva2V5d29yZD48a2V5d29yZD5NaWNyb3NhdGVsbGl0ZSBJbnN 0YWJpbGl0eTwv a2V5d29yZD48a2V5d29yZD5NaWNyb3NhdGVsbGl0ZSBSZXBIYXRzPC9rZX13b3Jk PjxrZX13b3Jk Pk1vbGVjdWxhciBTZXF1ZW5jZSBEYXRhPC9rZX13b3JkPjxrZX13b3JkPk51Y2x1YX IgUHJvdGVp bnMvKmdlbmV0aWNzPC9rZX13b3JkPjxrZX13b3JkPlBhbmNyZWFzL21ldGFib2xpc20 vcGF0aG9s b2d5PC9rZX13b3JkPjxrZX13b3JkP1N0b21hY2gvbWVOYWJvbGlzbS9wYXRob2xvZ3k 8L2tleXdv cmQ+PGtleXdvcmQ+U3RvbWFjaCBOZW9wbGFzbXMvKmdlbmV0aWNzL3BhdGhv bG9neTwva2V5d29y ZD48a2V5d29yZD5UcmFuc2NyaXBOaW9uIEZhY3RvcnMvKmdlbmV0aWNzPC9rZXl 3b3JkPjwva2V5 d29yZHM+PGRhdGVzPjx5ZWFyPjIwMTI8L311YXI+PHB1Yi1kYXR1cz48ZGF0ZT5K YW48L2RhdGU+PC9wdWItZGF0ZXM+PC9kYXRlcz48aXNibj4xMDk4LTEwMDQgKEVsZWN0cm9u aWMpJiN4RDsxMDU5 LTc3OTQgKExpbmtpbmcpPC9pc2JuPjxhY2Nlc3Npb24tbnVtPjIyMDA5OTQxPC9hY2 Nlc3Npb24t bnVtPjxlcmxzPjxyZWxhdGVkLXVybHM+PHVybD5odHRwOi8vd3d3Lm5jYmkubmx tLm5paC5nb3Yv cHVibWVkLzIyMDA50TQxPC91cmw+PC9yZWxhdGVkLXVybHM+PC91cmxzPjxjd XN0b20yPjMyNDA3 MTk8L2N1c3RvbTI+PGVsZWN0cm9uaWMtcmVzb3VyY2UtbnVtPjEwLjEwMDIvaH VtdS4yMTYzMzwv ZWxlY3Ryb25pYy1yZXNvdXJjZSiudWO+PC9yZWNvcmQ+PC9DaXRIPjwvRW5kT m90ZT4A 6. Six of the alterations in ARIDIA were either nonsense or frameshift alterations which were predicted to truncate the protein (Supplementary Table 4, Supplementary FIG. 1). Mutations in the MLL3 gene included a combination of nonsynonymous, nonsense, frameshift and splice-site mutations which occurred in amino acids predicted to be evolutionarily conserved (Supplementary Table 4, Supplementary FIG. 1). We found somatic frameshift and nonsynonymous sequence alterations in the related methyltransferases MLL or MLL2 in eight additional cases. Interestingly, no tumor had more than one gene mutated among the MLL genes suggesting that mutation in any one may be sufficient to confer a selective advantage in neoplastic cells.

### Example 3

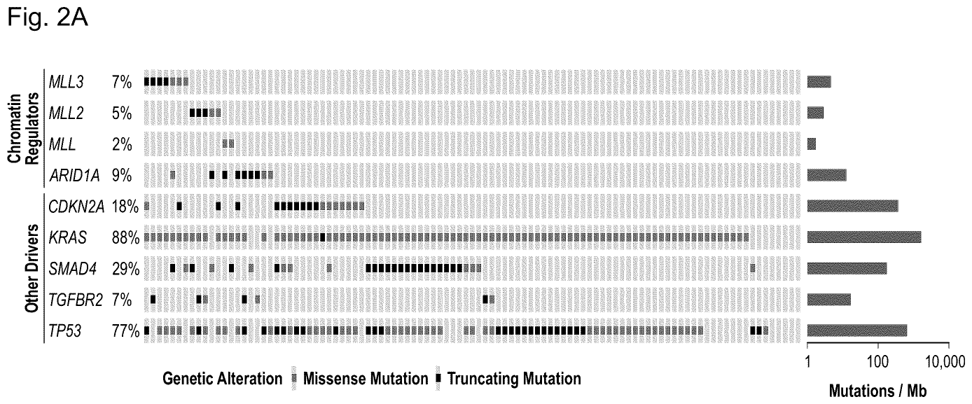

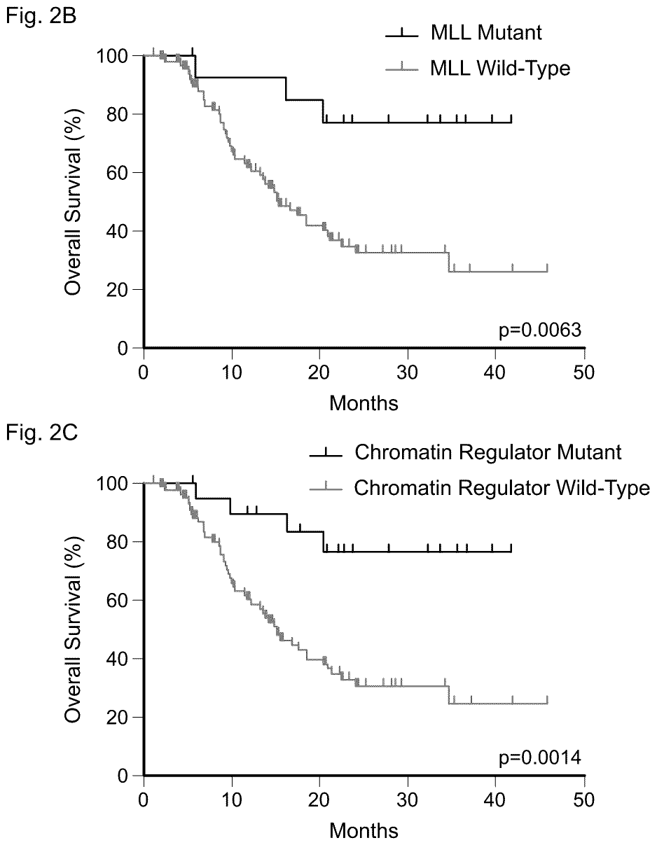

Given the global cellular changes regulated by chromatin regulators7,8, we examined the survival characteristics of patients with mutations in either the MLL or ARIDIA genes and found that patients with MLL alterations had a prolonged survival compared to those that were wild-type at these loci. Over three quarters (79%) of patients with mutations in MLL, MLL2 or MLL3 were still alive at the time of the analysis (median follow-up of 32 months), while the median survival in patients with wild-type sequences of these genes was 15.3 months (p=0.0063; log-rank test, FIG. 2A-2C and Supplementary FIG. 2). MLL mutation status was independent of clinical characteristics (Supplementary Table 7, p>0.05 for all comparisons by chi-square and unpaired t-test) and was found to be an independent prognostic factor (Supplementary Table 8, p=0.015, Cox multivariate regression analysis). Patients with alterations in ARIDIA had improved survival (p<0.05, log-rank test) but this observation was limited by a shorter follow-up time in affected individuals. Patients with mutations in any of the chromatin regulating genes identified had prolonged overall and progression-free survival and these observations were independent of other clinical variables (p<0.01 for all analyses, FIG. 2A-2C, Supplementary FIG. 2, and Supplementary Table 8). Genomic analyses in other tumor types have shown that somatic mutations of epigenetic regulators can have important clinical consequences, including improved outcome in patients with DAXX/ATRX alterations in pancreatic neuroendocrine tumorsPEVuZE5vdGU+PENpdGU+PEF1dGhvcj5KaWFvPC9BdXRob3I+PFllYXI+Mj AxMTwvWWVhcj48UmVj TnVtPjE2PC9SZWNOdW0+PERpc3BsYXIUZXhOPjxzdHlsZSBmYWNlPSJzdXBlcnNj cmlwdCI+OTwv c3R5bGU+PC9EaXNwbGF5VGV4dD48cmVjb3JkPjxyZWMtbnVtYmVyPjE2PC9yZW MtbnVtYmVyPjxm b3JlaWduLWtleXM+PGtleSBhcHA9IkVOIiBkYi1 pZDOid2R2ZDJ4MncOMmQ5eDNIe Hhhbng1ZHBl cmR1ZHBOdGEyeGZ4Ij4xNjwva2V5PjwvZm9yZWlnbi1rZXlzPjxyZWYtdH1wZSBuY W11PSJKb3Vy bmFsIEFydGljbGUiPjE3PC9yZWYtdH1wZT48Y29udHJpYnVOb3JzPjxhdXRob3JzPjxh dXRob3I+SmlhbywgWS48L2F1dGhvcj48YXV0aG9yPINoaSwgQy48L2F1dGhvcj48YXV0aG9yP kVkaWwsIEIu IEguPC9hdXRob3I+PGF1dGhvcj5kZSBXaWxkZ SwgUi4gRi48L2F1dGhvcj48YXVOaG 9yPktsaW1z dHJhLCBELiBTLjwvYXV0aG9yPjxhdXRob3I+TWFpdHJhLCBBLjwvYXV0aG9yPjx hdXRob3I+U2No dWxpY2ssIFIuIEQuPC9hdXRob3I+PGF1dGhvcj5UYW5nLCBMLiBILjwvYXV0aG9y PjxhdXRob3I+V29sZmdhbmcsIEMuIEwuPC9hdXRob3I+PGF1dGhvcj5DaG90aSwgTS4gQS48L2F1d Ghvcj48YXV0 aG9yP1Z1bGN1bGVzY3UsIFYuIEUuPC9hdXRob3I+PGF1dGhvcj5EaWF6LCBMLiBB LiwgSnIuPC9h dXRob3I+PGF1dGhvcj5Wb2dlbHNOZWIuLCBCLj wvYXV0aG9yPjxhdXRob3I+S21ue mxlciwgSy4g Vy48L2F1dGhvcj48YXV0aG9yPkhydWJhbiwgUi4gSC48L2F1dGhvcj48YXV0aG9yP1 BhcGFkb3Bv dWxvcywgTi48L2F1dGhvcj48L2F1dGhvcnM+PC9jb250cmlidXRvcnM+PGF1dGgtYW RkcmVzcz5M dWR3aWcgQ2VudGVyIGZvciBDYW5jZXIgR2VuZXRpY3MgYW5kIEhvd2FyZCBId WdoZXMgTWVkaWNh bCBJbnN0aXR1dGlvbnMslEpvaG5zlEhvcGtpbnMgS2ltbWVsIENhbmNlciBDZW50ZX IsIEJhbHRp bW9yZSwgTUQgMjEyMzEsIFVTQS48L2F1dGgtYWRkcmVzcz48dGl0bGVzPjx0aXR sZT5EQVhYLOFU UlgsIE1FTjEsIGFuZCBtVE9SIHBhdGh3YXkgZ2VuZXMgYXJ1IGZyZXF1ZW50bHkg YWxOZXJ1ZCBp biBwYW5jcmVhdGljIG5ldXJvZW5kb2NyaW51IHRbW9yczwvdGl0bGU+PHN1Y29u ZGFyeS1OaXRs ZT5TY211bmNlPC9zZWNvbmRhcnktdGl0bGU+PGFsdC1OaXRsZT5TY211bmN1PC9 hb HQtdGl0bGU+PC90aXRsZXM+PHBlcmlvZGljYWw+PGZ1bGwtdGl0bGU+U2NpZW5jZTwvZnVsb ClOaXRsZT48YWJi ci0xPlNjaWVuY2U8L2FiYnItMT48L3B1cmlvZGljYWw+PGFsdC1wZXJpb2RpY2FsPj xmdWxsLXRp dGxlPlNjaWVuY2U8L2Z1bGwtdGl0bGU+PGFiYnItMT5TY211bmN1PC9hYmJyLTE+PC9hbHQtcGVy aW9kaWNhbD48cGFnZXM+MTE50SOyMDM8L3BhZ2VzPjx2b2x1bWU+MzMxPC9 2b2xlbWU+PG51bWJ1 cj42MDIxPC9udW liZXI+PGtleXdvcmRzPjxrZX13b3JkPkFkYXBOb3IgUHJvdGVpbn MsIFNpZ25h bCBUcmFuc2R1Y2luZy8qZ2VuZXRpY3M8L2tleXdvcmQ+PGtleXdvcmQ+Q2FyY21u b21hLCBQYW5j cmVhdGljIER1Y3RhbC9nZW5ldGljczwva2V5d29yZD48a2V5d29yZD5DaHJvbWFOa W4gQXNzZWli bHkgYW5kIERpc2Fzc2VtYmx5L2dlbmV0aWNzPC9rZX13b3JkPjxrZX13b3JkPkROQS BIZWxpY2Fz ZXMvKmdlbmV0aWNzPC9rZX13b3JkPjxrZX13b3JkPkdlbmVzLCBUdW1vciBTdXBw cmVzc29yPC9r ZX13b3JkPjxrZX13b3JkPkh1bWFuczwva2V5d29yZD48a2V5d29yZD4qTXV0YXRpb24 8L2tleXdv cmQ+PGtleXdvcmQ+TmV1cm9lbmRvY3JpbmUgVHVtb3JzLypnZW5ldGljcy9tZXRh Ym9saXNtPC9r ZX13b3JkPjxrZX13b3JkPk51Y2xlYXIgUHJvdGVpbnMvKmdlbmV0aWNzPC9rZX13b3 JkPjxrZX13 b3JkP1BURU4gUGhvc3Bob2h5ZHJvbGFzZS9nZW5ldGljczwva2V5d29yZD48a2V5d2 9yZD5QYW5j cmVhdGljIE51b3BsYXNtcy8qZ2VuZXRpY3MvbWVOYWJvbGlzbTwva2V5d29yZD48 a2V5d29yZD5Q aG9zcGhhdGlkeWxpbm9zaXRvbCAzLUtpbmFzZXMvZ2VuZXRpY3M8L2tleXdvcmQ+PGtleXdvcmQ+UHJvZ25vc21zPC9rZX13b3JkPjxrZX13b3JkPlByb3RvLU9uY29nZW51IFByb3R1aW5zL ypnZW5l dGljczwva2V5d29yZD48a2V5d29yZD5TZXF1ZW5jZ SBBbmFseXNpcywgRE5BPC9r ZX13b3JkPjxr ZX13b3JkP1NpZ25hbCBUcmFuc2R1Y3Rpb24vZ2VuZXRpY3M8L2tleXdvcmQ+PGtle XdvcmQ+VE9S IFNlcmluZS1UaHJlb25pbmUgS2luYXNlcy9nZW5ldGljcy8qbWVOYWJvbGlzbTwva2 V5d29yZD48 a2V5d29yZD5UdW1vciBTdXBwcmVzc29yIFByb3RaW5zL2dlbmV0aWNzPC9rZX13 b3JkPjwva2V5 d29yZHM+PGRhdGVzPjx5ZWFyPjIwMTE8L311YXI+PHB1Yi1kYXR1cz48ZGF0ZT5 NYXIgNDwvZGFO ZT48L3B1Yi1kYXRlcz48L2RhdGVzPjxpc2JuPjEwOTUtOTIwMyAoRWx1Y3Ryb25p YykmI3hEOzAw MzYtODA3NSAoTGlua2luZyk8L21zYm4+PGFjY2Vzc2lvbiludWO+MjEyNTIzMTU8 L2FjY2Vzc2lv bi1udW0+PHVybHM+PHJ1bGFOZWQtdXJscz48dXJsPmhOdHA6Ly93d3cubmNiaS5ub GOubmloLmdv di9wdWJtZWQvMjEyNTIzMTU8L3VybD48L3J1bGFOZWQtdXJscz48L3VybHM+PG N1c3RvbTI+MzEO NDQ5NjwvY3VzdG9tMj48ZWxlY3Ryb25pYy1yZXNvdXJjZS1udWO+MTAuMTEyNi 9zY211bmNlLjEy MDA2MDk8L2VsZWN0cm9uaWMtcmVzb3VyY2UtbnVtPjwvcmVjb3JkPjwvQ210ZT4 8L0VuZE5vdGU+PEVuZE5vdGU+PENpdGU+PEF1dGhvcj5KaWFvPC9BdXRob3I+PFIIYXI+MjAxMT wvWWVhcj48UmVj TnVtPjE2PC9SZWNOdW0+PERpc3BsYX1UZXhOPjxzdHlsZSBmYWN1PSJzdXBlcnNj cmlwdCI+OTwv c3R5bGU+PC9EaXNwbGF5VGV4dD48cmVjb3JkPjxyZWMtbnVtYmVyPjE2PC9yZW MtbnVtYmVyPjxm b3JlaWduLWtleXM+PGtleSBhcHA9IkVOIiBkYi1 pZDOid2R2ZDJ4MncOMmQ5eDNIe Hhhbng1ZHBl cmR1ZHBOdGEyeGZ4Ij4xNjwva2V5PjwvZm9yZWlnbi1rZXlzPjxyZWYtdH1wZSBuY W11PSJKb3Vy bmFsIEFydGljbGUiPjE3PC9yZWYtdH1wZT48Y29udHJpYnVOb3JzPjxhdXRob3JzPjxh dXRob3I+SmlhbywgWS48L2F1dGhvcj48YXV0aG9yPINoaSwgQy48L2F1dGhvcj48YXV0aG9yP kVkaWwsIEIu IEguPC9hdXRob3I+PGF1dGhvcj5kZSBXaWxkZ SwgUi4gRi48L2F1dGhvcj48YXVOaG 9yPktsaW1z dHJhLCBELiBTLjwvYXV0aG9yPjxhdXRob3I+TWFpdHJhLCBBLjwvYXV0aG9yPjx hdXRob3I+U2No dWxpY2ssIFIuIEQuPC9hdXRob3I+PGF1dGhvcj5UYW5nLCBMLiBILjwvYXV0aG9y PjxhdXRob3I+V29sZmdhbmcsIEMuIEwuPC9hdXRob3I+PGF1dGhvcj5DaG90aSwgT S4gQ S48L2F1d Ghvcj48YXV0 aG9yP1Z1bGN1bGVzY3UsIFYuIEUuPC9hdXRob3I+PGF1dGhvcj5EaWF6LCBMLiBB LiwgSnIuPC9h dXRob3I+PGF1dGhvcj5Wb2dlbHNOZWIuLCBCLj wvYXV0aG9yPjxhdXRob3I+S21ue mxlciwgSy4g Vy48L2F1dGhvcj48YXV0aG9yPkhydWJhbiwgUi4gSC48L2F1dGhvcj48YXV0aG9yP1 BhcGFkb3Bv dWxvcywgTi48L2F1dGhvcj48L2F1dGhvcnM+PC9jb250cmlidXRvcnM+PGF1dGgtYW RkcmVzcz5M dWR3aWcgQ2VudGVyIGZvciBDYW5jZXIgR2VuZXRpY3MgYW5kIEhvd2FyZCBId WdoZXMgTWVkaWNh bCBJbnN0aXR1dGlvbnMslEpvaG5zlEhvcGtpbnMgS2ltbWVsIENhbmNlciBDZW50ZX IsIEJhbHRp bW9yZSwgTUQgMjEyMzEsIFVTQS48L2F1dGgtYWRkcmVzcz48dGl0bGVzPjx0aXR sZT5EQVhYLOFU UlgsIE1FTjEsIGFuZCBtVE9SIHBhdGh3YXkgZ2VuZXMgYXJ1IGZyZXF1ZW50bHkg YWxOZXJ1ZCBp biBwYW5jcmVhdGljIG5ldXJvZW5kb2NyaW51IHRbW9yczwvdGl0bGU+PHN1Y29u ZGFyeS1OaXRs ZT5TY211bmN1PC9zZWNvbmRhcnktdGl0bGU+PGFsdC1OaXRsZT5TY211bmN1PC9 hb HQtdGl0bGU+PC90aXRsZXM+PHBlcmlvZGljYWw+PGZ1bGwtdGl0bGU+U2NpZW5jZTwvZnVsb C10aXRsZT48YWJi ci0xPlNjaWVuY2U8L2FiYnItMT48L3B1cmlvZGljYWw+PGFsdC1wZXJpb2RpY2FsPj xmdWxsLXRp dGxlPlNjaWVuY2U8L2Z1bGwtdGl0bGU+PGFiYnItMT5TY211bmN1PC9hYmJyLTE+PC9hbHQtcGVy aW9kaWNhbD48cGFnZXM+MTE50SOyMDM8L3BhZ2VzPjx2b2x1bWU+MzMxPC9 2b2xlbWU+PG51bWJ1 cj42MDIxPC9udW liZXI+PGtleXdvcmRzPjxrZX13b3JkPkFkYXB0b3IgUHJvdGVpbn MsIFNpZ25h bCBUcmFuc2R1Y2luZy8qZ2VuZXRpY3M8L2tleXdvcmQ+PGtleXdvcmQ+Q2FyY21u b21hLCBQYW5j cmVhdGljIER1Y3RhbC9nZW5ldGljczwva2V5d29yZD48a2V5d29yZD5DaHJvbWFOa W4gQXNzZWli bHkgYW5kIERpc2Fzc2VtYmx5L2dlbmV0aWNzPC9rZX13b3JkPjxrZX13b3JkPkROQS BIZWxpY2Fz ZXMvKmdlbmV0aWNzPC9rZX13b3JkPjxrZX13b3JkPkdlbmVzLCBUdW1vciBTdXBw cmVzc29yPC9r ZX13b3JkPjxrZX13b3JkPkhlbWFuczwva2V5d29yZD48a2V5d29yZD4qTXV0YXRpb24 8L2tleXdv cmQ+PGtleXdvcmQ+TmV1cm9lbmRvY3JpbmUgVHVtb3JzLypnZW5ldGljcy9tZXRh Ym9saXNtPC9r ZX13b3JkPjxrZX13b3JkPk51Y2xlYXIgUHJvdGVpbnMvKmdlbmV0aWNzPC9rZX13b3 JkPjxrZX13 b3JkP1BURU4gUGhvc3Bob2h5ZHJvbGFzZS9nZW5ldGljczwva2V5d29yZD48a2V5d2 9yZD5QYW5j cmVhdGljIE51b3BsYXNtcy8qZ2VuZXRpY3MvbWVOYWJvbGlzbTwva2V5d29yZD48 a2V5d29yZD5Q aG9zcGhhdGlkeWxpbm9zaXRvbCAzLUtpbmFzZXMvZ2VuZXRpY3M8L2tleXdvcmQ+PGtleXdvcmQ+UHJvZ25vc21zPC9rZX13b3JkPjxrZX13b3JkPlByb3RvLU9uY29nZW51IFByb3R1aW5zL ypnZW5l dGljczwva2V5d29yZD48a2V5d29yZD5TZXF1ZW5jZ SBBbmFseXNpcywgRE5BPC9r ZX13b3JkPjxr ZX13b3JkP1NpZ25hbCBUcmFuc2R1Y3Rpb24vZ2VuZXRpY3M8L2tleXdvcmQ+PGtle XdvcmQ+VE9S IFNlcmluZS1UaHJlb25pbmUgS2luYXNlcy9nZW5ldGljcy8qbWVOYWJvbGlzbTwva2 V5d29yZD48 a2V5d29yZD5UdW1vciBTdXBwcmVzc29yIFByb3RaW5zL2dlbmV0aWNzPC9rZX13 b3JkPjwva2V5 d29yZHM+PGRhdGVzPjx5ZWFyPjIwMTE8L311YXI+PHB1Yi1kYXR1cz48ZGF0ZT5 NYXIgNDwvZGFO ZT48L3B1Yi1kYXRlcz48L2RhdGVzPjxpc2JuPjEwOTUtOTIwMyAoRWx1Y3Ryb25p YykmI3hEOzAw MzYtODA3NSAoTGlua2luZyk8L21zYm4+PGFjY2Vzc2lvbi1udWO+MjEyNTIzMTU8 L2FjY2Vzc2lv bi1udWO+PHVybHM+PHJ1bGFOZWQtdXJscz48dXJsPmhOdHA6Ly93d3cubmNiaS5ub GOubmloLmdv di9wdWJtZWQvMjEyNTIzMTU8L3VybD48L3J1bGFOZWQtdXJscz48L3VybHM+PG Nic3RvbTI+MzEO NDQ5NjwvY3VzdG9tMj48ZWxlY3Ryb25pYy1yZXNvdXJjZS1udWO+MTAuMTEyNi 9zY211bmNlLjEy MDA2MDk8L2VsZWN0cm9uaWMtcmVzb3VyY2UtbnVtPjwvcmVjb3JkPjwvQ210ZT4 8L0VuZE5vdGU+9, and a decreased survival in patients with ARIDIA and ARIDIB mutations in neuroblastomaPEVuZE5vdGU+PENpdGU+PEF1dGhvcj5TYXVzZW48LOF1dGhvcj48 WWVhcj4yMDEzPC9ZZWFyPjxS ZWNOdW0+NTwvUmVjTnVtPjxEaXNwbGF5VGV4dD48c3R5bGUgZmFjZT0ic3Vw ZXJzY3JpcHQiPjEw PC9zdHlsZT48L0Rpc3BsYX1UZXhOPjxyZWNvcmQ+PHJlYyludWliZXI+NTwvcmVj LW5lbWJlcj48 Zm9yZWlnbi1rZXlzPjxrZXkgYXBwPSJFTiIgZGItaWQ9IndkdmQyeDJ3NDJkOXgzZX h4YW54NWRw ZXJkZWRwdHRhMnhmeCI+NTwva2V5PjwvZm9yZWlnbilrZXlzPjxyZWYtdHlwZSB uYWllPSJKb3Vy bmFsIEFydGljbGUiPjE3PC9yZWYtdHlwZT48Y29udHJpYnVOb3JzPjxhdXRob3JzPjxh dXRob3I+U2F1c2VuLCBNLjwvYXV0aG9yPjxhdXRob3I+TGVhcnksIFIuIEouPC9hdXRob3I+P GF1dGhvcj5K b251cywgUy48L2F1dGhvcj48YXV0aG9yPld1LCBKLjwvYXV0aG9yPjxhdXRob3I+U mV5bm9sZHMs IEMuIFAuPC9hdXRob3I+PGF1dGhvcj5MaXUsIFguPC9hdXRob3I+PGF1dGhvcj5CbG Fja2ZvcmQs IEEuPC9hdXRob3I+PGF1dGhvcj5QYXJtaWdpYW5pLCBHLjwvYXV0aG9yPjxhdXR ob3I+RGlheiwg TC4gQS4sIEpyLjwvYXV0aG9yPjxhdXRob3I+UGFwYWRvcG91bG9zLCBOLjwvYX V0aG9yPjxhdXRo b3I+Vm9nZWxzdGVpbiwgQi48L2F1dGhvcj48YXV0aG9yPktpbnpsZXIsIEsuIFcuPC9h dXRob3I+PGF1dGhvcj5WZWxjdWxlc2N1LCBWLiBFLjwvYXV0aG9yPjxhdXRob3I+SG9nYXJ 0eSwgTS4gRC48 L2F1dGhvcj48L2F1dGhvcnM+PC9jb250cmlidXRvcnM+PGF1dGgtYWRkcmVzcz5Md WR3aWcgQ2Vu dGVyIGZvciBDYW5jZXIgR2VuZXRpY3MgYW5kIFRoZXJhcGV1dGljcywgVGhlIFN pZG5leSBLaW lt ZWwgQ29tcHJlaGVuc212ZSBDYW5jZXIgQ2VudGVyIGFOIEpvaG5zIEhvcGtpbnMsI EJhbHRpbW9y ZSwgTWFyeWxhbmQsIFVTQS48L2F1dGgtYWRkcmVzcz48dGl0bGVzPjx0aXRsZT5 JbnR1Z3JhdGVk IGdlbm9taWMgYW5hbH1zZXMgaWRlbnRpZnkgQVJJRDFBIGFuZCBBUklEMUIgY WxOZXJhdGlvbnMg aW4gdGhlIGNoaWxkaG9vZCBjYW5jZXIgbmV1cm9ibGFzdG9tYTwvdGl0bGU+PHN IY29uZGFyeS10 aXRsZT50YXQgR2VuZXQ8L3N1Y29uZGFyeS1OaXRsZT48YWx0LXRpdGxlPk5hdH VyZSBnZW5ldGlj czwvYWx0LXRpdGxlPjwvdGl0bGVzPjxwZXJpb2RpY2FsPjxmdWxsLXRpdGxlPk5hd CBHZW5ldDwv ZnVsbC1OaXRsZT48YWJici0xPk5hdHVyZSBnZW5ldGljczwvYWJici0xPjwvcGVyaW 9kaWNhbD48 YWx0LXBlcmlvZGljYWw+PGZ1bGwtdGl0bGU+TmF0IEdlbmVOPC9mdWxsLXRpd GxlPjxhYmJyLTE+TmF0dXJIIGdlbmV0aWNzPC9hYmJyLTE+PC9hbHQtcGVyaW9kaWNhbD48cGFnZX M+MTItNzwvcGFn ZXM+PHZvbHVtZT40NTwvdm9sdWllPjxudW1iZXI+MTwvbnVtYmVyPjxrZX13b3Jk cz48a2V5d29y ZD5DZWxsIExpbmUsIFR1bW9yPC9rZX13b3JkPjxrZX13b3JkPkNoaWxkPC9rZX13b3J kPjxrZX13 b3JkPkROQSBDb3B5IE51bWJlciBWYXJpYXRpb25zPC9rZX13b3JkPjxrZX13b3JkPkR OQS1CaW5k aW5nIFByb3RlaW5zLypnZW5ldGljczwva2V5d29yZD48a2V5d29yZD5FeG9tZTwva2 V5d29yZD48 a2V5d29yZD5HZW51IEV4cHJlc3Npb24gUHJvZmlsaW5nPC9rZX13b3JkPjxrZX13b3Jk PkdlbmUg T3JkZXI8L2tleXdvcmQ+PGtleXdvcmQ+R2Vub21pY3M8L2tleXdvcmQ+PGtleXdvcm Q+SHVtYW5z PC9rZX13b3JkPjxrZX13b3JkPipNdXRhdGlvbjwva2V5d29yZD48a2V5d29yZD5OZXVy b2JsYXN0 b21hLypnZW5ldGljcy9tb3JOYWxpdHk8L2tleXdvcmQ+PGtleXdvcmQ+TnVjbGVhciB Qcm90ZWlu cy8qZ2VuZXRpY3M8L2tleXdvcmQ+PGtleXdvcmQ+U2VxdWVuY2UgQW5hbHlzaX MsIEROQTwva2V5 d29yZD48a2V5d29yZD5UcmFuc2NyaXBOaW9uIEZhY3RvcnMvKmdlbmV0aWNzPC 9rZX13b3JkPjxr ZX13b3JkP1R1bW9yIE1hcmtlcnMslEJpb2xvZ2ljYWwvZ2VuZXRpY3M8L2tleXdvcmQ+PC9rZX13 b3Jkcz48ZGF0ZXM+PHIIYXI+MjAxMzwveWVhcj48cHViLWRhdGVzPjxkYXRlPkph bjwvZGF0ZT48 L3B1Yi1kYXR1cz48L2RhdGVzPjxpc2JuPjE1NDYtMTcxOCAoRWx1Y3Ryb25pYykmI 3hEOzEwNjEt NDAzNiAoTGlua2luZyk8L2lzYm4+PGFjY2Vzc2lvbiludWO+MjMyMDIxMjg8L2FjY 2Vzc2lvbilu dW0+PHVybHM+PHJbGFOZWQtdXJscz48dXJsPmhOdHA6Ly93d3cubmNiaS5ubGOu bmloLmdvdi9w dWJtZWQvMjMyMDIxMjg8L3VybD48L3J1bGFOZWQtdXJscz48L3VybHM+PGN1c3 RvbTI+MzU1Nzk1 OTwvY3VzdG9tMj48ZWxlY3Ryb25pYy lyZXNvdXJjZSludWO+MTAuMTAzOC9uZ y4yNDkzPC9lbGVj dHJvbmljLXJlc291cmN1LW51bT48L3J1Y29yZD48LONpdGU+PC9FbmROb3R1PgB=PEVuZE5vdGU+PENpdGU+PEF1dGhvcj5TYXVzZW48LOF1dGhvcj48WWVhcj4yM DEzPC9ZZWFyPjxS ZWNOdW0+NTwvUmVj TnVtPjxEaXNwbGF5VGV4dD48c3R5bGUgZmFjZT0ic3Vw ZXJzY3JpcHQiPjEw PC9zdH1sZT48L0Rpc3BsYX1UZXhOPjxyZWNvcmQ+PHJ1YyludWliZXI+NTwvcmVj LW5lbWJlcj48 Zm9yZWlnbi1rZXlzPjxrZXkgYXBwPSJFTiIgZGItaWQ9IndkdmQyeDJ3NDJkOXgzZX h4YW54NWRw ZXJkZWRwdHRhMnhmeCI+NTwva2V5PjwvZm9yZWlnbi1rZXlzPjxyZWYtdH1wZSB uYW11PSJKb3Vy bmFsIEFydGljbGUiPjE3PC9yZWYtdH1wZT48Y29udHJpYnVOb3JzPjxhdXRob3JzPjxh dXRob3I+U2F1c2VuLCBNLjwvYXV0aG9yPjxhdXRob3I+TGVhcnksIFIuIEouPC9hdXRob3I+P GF1dGhvcj5K b251cywgUy48L2F1dGhvcj48YXV0aG9yPld1LCBKLjwvYXV0aG9yPjxhdXRob3I+U mV5bm9sZHMs IEMuIFAuPC9hdXRob3I+PGF1dGhvcj5MaXUsIFguPC9hdXRob3I+PGF1dGhvcj5CbG Fja2ZvcmQs IEEuPC9hdXRob3I+PGF1dGhvcj5QYXJtaWdpYW5pLCBHLjwvYXV0aG9yPjxhdXR ob3I+RGlheiwg TC4gQ S4sIEpyLjwvYXV0aG9yPjxhdXRob3I+UGFwYWRvcG91bG9zLCBOLjwvYX V0aG9yPjxhdXRo b3I+Vm9nZWxzdGVpbiwgQi48L2F1dGhvcj48YXV0aG9yPktpbnpsZXIsIEsuIFcuPC9h dXRob3I+PGF1dGhvcj5WZWxjdWxlc2N1LCBWLiBFLjwvYXV0aG9yPjxhdXRob3I+SG9nYXJ 0eSwgTS4gRC48 L2F1dGhvcj48L2F1dGhvcnM+PC9jb250cmlidXRvcnM+PGF1dGgtYWRkcmVzcz5Md WR3aWcgQ2Vu dGVyIGZvciBDYW5jZXIgR2VuZXRpY3MgYW5kIFRoZXJhcGV1dGljcywgVGhlIFN pZG5leSBLaW lt ZWwgQ29tcHJlaGVuc212ZSBDYW5jZXIgQ2VudGVyIGFOIEpvaG5zIEhvcGtpbnMsI EJhbHRpbW9y ZSwgTWFyeWxhbmQsIFVTQS48L2F1dGgtYWRkcmVzcz48dGl0bGVzPjx0aXRsZT5 JbnR1Z3JhdGVk IGdlbm9taWMgYW5hbH1zZXMgaWRlbnRpZnkgQVJJRDFBIGFuZCBBUk1EMUIgY WxOZXJhdGlvbnMg aW4gdGhlIGNoaWxkaG9vZCBjYW5jZXIgbmV1cm9ibGFzdG9tYTwvdGl0bGU+PHN 1Y29uZGFyeS10 aXRsZT50YXQgR2VuZXQ8L3N1Y29uZGFyeS1OaXRsZT48YWx0LXRpdGxlPk5hdH VyZSBnZW5ldGlj czwvYWx0LXRpdGxlPjwvdGl0bGVzPjxwZXJpb2RpY2FsPjxmdWxsLXRpdGxlPk5hd CBHZW5ldDwv ZnVsbC1OaXRsZT48YWJici0xPk5hdHVyZSBnZW5ldGljczwvYWJici0xPjwvcGVyaW 9kaWNhbD48 YWx0LXBlcmlvZGljYWw+PGZ1bGwtdGl0bGU+TmFOIEdlbmVOPC9mdWxsLXRpd GxlPjxhYmJyLTE+TmF0dXJ1IGdlbmV0aWNzPC9hYmJyLTE+PC9hbHQtcGVyaW9kaWNhbD48cGFnZX M+MTItNzwvcGFn ZXM+PHZvbHVtZT40NTwvdm9sdWllPjxudW1iZXI+MTwvbnVtYmVyPjxrZX13b3Jk cz48a2V5d29y ZD5DZWxsIExpbmUsIFR1bW9yPC9rZX13b3JkPjxrZX13b3JkPkNoaWxkPC9rZX13b3J kPjxrZX13 b3JkPkROQSBDb3B5IE51bWJlciBWYXJpYXRpb25zPC9rZX13b3JkPjxrZX13b3JkPkR OQS1CaW5k aW5nIFByb3RlaW5zLypnZW5ldGljczwva2V5d29yZD48a2V5d29yZD5FeG9tZTwva2 V5d29yZD48 a2V5d29yZD5HZW51IEV4cHJlc3Npb24gUHJvZmlsaW5nPC9rZX13b3JkPjxrZX13b3Jk PkdlbmUg T3JkZXI8L2tleXdvcmQ+PGtleXdvcmQ+R2Vub21pY3M8L2tleXdvcmQ+PGtleXdvcm Q+SHVtYW5z PC9rZX13b3JkPjxrZX13b3JkPipNdXRhdGlvbjwva2V5d29yZD48a2V5d29yZD5OZXVy b2JsYXN0 b21hLypnZW5ldGljcy9tb3JOYWxpdHk8L2tleXdvcmQ+PGtleXdvcmQ+TnVjbGVhciB Qcm90ZWlu cy8qZ2VuZXRpY3M8L2tleXdvcmQ+PGtleXdvcmQ+U2VxdWVuY2UgQW5hbHlzaX MsIEROQTwva2V5 d29yZD48a2V5d29yZD5UcmFuc2NyaXBOaW9uIEZhY3RvcnMvKmdlbmV0aWNzPC 9rZX13b3JkPjxr ZX13b3JkPlR1bW9yIE1hcmtlcnMslEJpb2xvZ2ljYWwvZ2VuZXRpY3M8L2tleXdvcmQ+PC9rZX13 b3Jkcz48ZGF0ZXM+PHllYXI+MjAxMzwveWVhcj48cHViLWRhdGVzPjxkYXRlPkph bjwvZGF0ZT48 L3B1Yi1kYXRlcz48L2RhdGVzPjxpc2JuPjE1NDYtMTcxOCAoRWxlY3Ryb25pYykmI 3hEOzEwNjEt NDAzNiAoTGlua2luZyk8L2lzYm4+PGFjY2Vzc2lvbiludWO+MjMyMDIxMjg8L2FjY 2Vzc2lvbilu dWO+PHVybHM+PHJlbGFOZWQtdXJscz48dXJsPmhOdHA6Ly93d3cubmNiaS5ubGOu bmloLmdvdi9w dWJtZWQvMjMyMDIxMjg8L3VybD48L3JbGFOZWQtdXJscz48L3VybHM+PGN1c3 RvbTI+MzUlNzk1 OTwvY3VzdG9tMj48ZWxlY3Ryb25pYy lyZXNvdXJjZSludWO+MTAuMTAzOC9uZ y4yNDkzPC9lbGVj dHJvbmljLXJlc291cmNlLW51bT48L3JlY29yZD48LONpdGU+PC9FbmROb3RlPgB=10.

### Example 4

In parallel to the sequencing analyses of neoplastic tissues, we evaluated the utility of using somatic mutations in circulating tumor DNA (ctDNA) to identify patients likely to recur after surgical intervention. Through sequencing analyses of tumor samples we identified somatic mutations that could be used to detect ctDNA in 51 patients from whom plasma was available, largely focusing on alterations in the KRAS, BRAF and PIK3CA genes (Online Methods). Using digital polymerase chain reaction (dPCR) approaches, we were able to demonstrate that these alterations were detectable in the plasma of 22 patients (43%) at the time of diagnosis, with a specificity of >99.9% (Example 1). Consistent with recent reports11,12, these results suggest that a significant fraction of early stage pancreatic cancers could be diagnosed non-invasively using approaches that focus on a few specific genetic alterations.

### Example 5

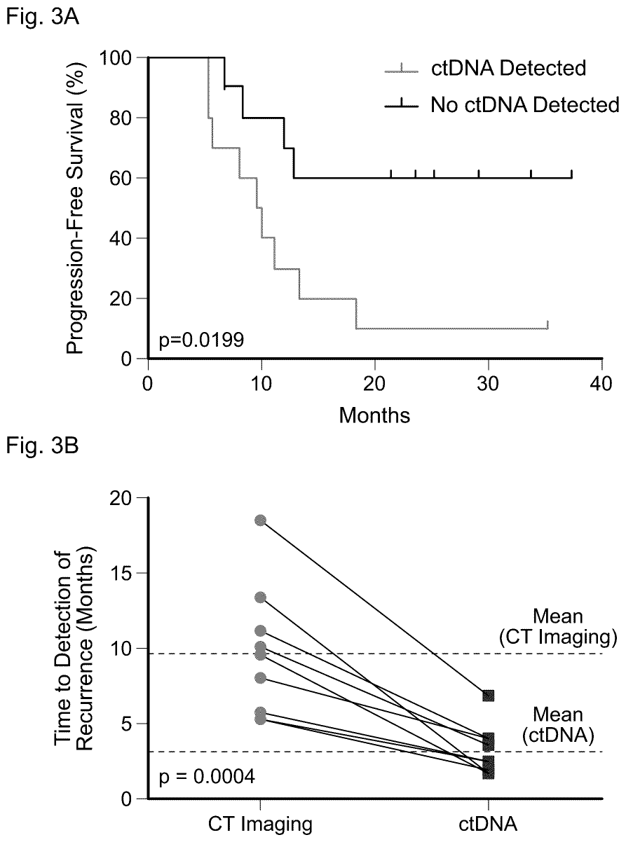

dPCR analyses were performed using plasma samples obtained at various time points after surgical resection. These analyses revealed that patients with detectable ctDNA in their plasma were more likely to relapse than those with undetectable alterations (p=0.02, log-rank test, FIG. 3a). Disease progression using ctDNA was detected at an average of 3.1 months after surgery compared to 9.6 months using standard CT imaging (p=0.0004, paired t-test, FIG. 3b). The presence of ctDNA at the time of diagnosis also provided a predictor of disease recurrence (p=0.015, log-rank test, Supplementary FIG. 3). These analyses suggest that tests to detect sequence alterations in cell free DNA may provide a highly specific approach for early detection of residual or recurrent disease after surgical resection.

### Example 6

Given the poor outcome and limited therapeutic options for patients with pancreatic cancer, we investigated whether mutations observed in individual cases may be clinically actionable using existing or investigational therapies. We examined genetic alterations that were associated with 1) FDA-approved therapies for oncologic indications, 2) therapies in published prospective clinical studies, and 3) ongoing clinical trials for patients with pancreatic cancer or other tumor types. We also evaluated alterations in five genes in the patients' germline that may affect cancer predisposition as detection of such changes have important implications for early detection and interventionPEVuZE5vdGU+PENpdGU+PEF1dGhvcj5JYWNvYnV6aW8tRG9uYWh1 ZTwvQXV0aG9yPjxZZWFyPjIw MTI8L111YXI+PFJlY051bT43PC9SZWNOdW0+PERpc3BsYX1UZXhOPjxzdHlsZSBm YWN1PSJzdXBl cnNjcmlwdCI+MTM8L3N0eWxlPjwvRGlzcGxheVRleHQ+PHJlY29yZD48cmVjLW51 bWJlcj43PC9y ZWMtbnVtYmVyPjxmb3JlaWduLWtleXM+PGtleSBhcHA9IkVOIiBkYi1 pZDOid2R2Z DJ4MncOMmQ5 eDNleHhhbng1ZZHB1cmR1ZHBOdGEyeGZ4Ij43PC9rZXk+PC9mb3J1aWduLWtleXM+P HJIZilOeXBI IG5hbWU9IkpvdXJuYWwgQXJOaWNsZSI+MTc8L3JIZi1OeXBIPjxjb250cmlidXRvcn M+PGF1 dGhv cnM+PGF1dGhvcj5JYWNvYnV6aW8tRG9uYWh1ZSwgQy4gQS48L2F1dGhvcj48YX V0aG9yP1Z1bGN1 bGVzY3UsIFYuIEUuPC9hdXRob3I+PGF1dGhvcj5Xb2xmZ2FuZywgQy4gTC48L2F1d Ghvcj48YXV0 aG9yPkhydWJhbiwgUi4gSC48L2F1dGhvcj48L2F1dGhvcnM+PC9jb250cmlidXRvcnM+PGF1 dGgt YWRkcmVzcz5EZXBhcnRtZW50IG9mIFBhdGhvbG9neSwgSm9obnMgSG9wa2lucyB Vbml2ZXJzaXR5 IFNjaG9vbCBvZiBNZWRpY2luZSwgQmFsdGltb3JILCBNRCwgVVNBLjwvYXVOaC 1hZGRyZXNzPjx0 aXRsZXM+PHRpdGxlPkdlbmVOaWMgYmFzaXMgb2YgcGFuY3JIYXMgY2FuY2VyI GRIdmVsb3BtZW50 IGFuZCBwcm9ncmVzc2lvbjogaW5zaWdodHMgZnJvbSB3aG9sZSIleG9tZSBhbmQgd 2hvbGUtZ2Vu b211IHNlcXVlbmNpbmc8L3RpdGxlPjxzZWNvbmRhcnktdGl0bGU+Q2xpbiBDYW5jZ XIgUmVzPC9z ZWNvbmRhcnktdGl0bGU+PGFsdC1OaXRsZT5DbGluaWNhbCBjYW5jZXIgcmVzZW FyY2ggOiBhbiBv ZmZpY2lhbCBqb3VybmFsIG9mIHRoZSBBbWVyaWNhbiBBc3NvY21hdGlvbiBmb3Ig Q2FuY2VyIFJ1 c2VhcmNoPC9hbHQtdGl0bGU+PC90aXRsZXM+PHBlcmlvZGljYWw+PGZ1bGwtdGl 0bGU+Q2xpbiBD YW5jZXIgUmVzPC9mdWxsLXRpdGxlPjxhYmJyLTE+Q2xpbmljYWwgY2FuY2VyIH Jlc2VhcmNoIDog YW4gb2ZmaWNpYWwgam91cm5hbCBvZiBOaGUgQW1lcmljYW4gQXNzb2NpYXR pb24gZm9yIENhbmN1 ciBSZXNYXJjaDwvYWJici0xPjwvcGVyaW9kaWNhbD48YWx0LXBlcmlvZGljYWw+PGZ1bGwtdGl0 bGU+Q2xpbiBDYW5jZXIgUmVzPC9mdWxsLXRpdGxlPjxhYmJyLTE+Q2xpbmljYW wgY2FuY2VyIHJI c2VhcmNolDogYW4gb2ZmaWNpYWwgam91cm5hbCBvZiBOaGUgQWllcmljYW4g QXNzb2NpYXRpb24g Zm9yIENhbmNlciBSZXN1YXJjaDwvYWJiciOxPjwvYWx0LXBlcmlvZGljYWw+PHBh Z2VzPjQyNTct NjU8L3BhZ2VzPjx2b2x1bWU+MTg8L3ZvbHVtZT48bnVtYmVyPjE2PC9udW liZXI+PGtleXdvcmRz PjxrZX13b3JkPkNhcmNpbm9tYSwgUGFuY3J1YXRpYyBEdWN0YWwvZ2VuZXRpY 3MvcGF0aG9sb2d5 PC9rZX13b3JkPjxrZX13b3JkPkRpc2Vhc2UgUHJvZ3Jlc3Npb248L2tleXdvcmQ+PGtleX dvcmQ+RmFtaWx5PC9rZX13b3JkPjxrZX13b3JkPkdlbm9tZSwgSHVtYW48L2tleXdvcmQ+PGt1 eXdvcmQ+R2VybS1MaW51IE11dGF0aW9uPC9rZX13b3JkPjxrZX13b3JkPkh1bWFuczwva2V5d29 yZD48a2V5 d29yZD5OZW9wbGFzbSBJbnZhc212ZW5lc3M8L2tleXdvcmQ+PGtleXdvcmQ+TmVv cGxhc20gTWV0 YXNOYXNpczwva2V5d29yZD48a2V5d29yZD5QYW5jcmVhdGljIE51b3BsYXNtcy8q Z2VuZXRpY3Mv KnBhdGhvbG9neTwva2V5d29yZD48a2V5d29yZD5TZXF1ZW5jZSBBbmFseXNpcyw gRE5BPC9rZX13 b3JkPjwva2V5d29yZHM+PGRhdGVzPjx5ZWFyPjIwMTI8L311YXI+PHB1Yi1kYXRlc z48ZGF0ZT5B dWcgMTU8L2RhdGU+PC9wdWItZGF0ZXM+PC9kYXRlcz48aXNibj4xMDc4LTAOM zIgKFByaW50KSYj eEQ7MTA30COwNDMyIChMaW5raW5nKTwvaXNibj48YWNjZXNzaW9uLW51bT4 yMjg5NjY5MjwvYWNj ZXNzaW9uLW51bT48dXJscz48cmVsYXR1ZC11cmxzPjxl cmw+aHROcDovL3d3dy5u Y2JpLm5sbS5u aWguZ292L3B1Ym1lZC8yMjg5NjY5MjwvdXJsPjwvcmVsYXR1ZC11cmxzPjwvdXJsc z48Y3VzdG9t Mj4zNDIyNzcxPC9jdXN0b20yPjxlbGVjdHJvbmljLXJlc291cmNILW51bT4xMC4xMT U4LzEwNzgt MDQzMi5DQ1ItMTItMDMxNTwvZWxlY3Ryb25pYylyZXNvdXJjZSludWO+PC9yZ WNvcmQ+PC9DaXR1 PjwvRW5kTm90ZT4A PEVuZE5vdGU+PENpdGU+PEF1dGhvcj5JYWNvYnV6aW8tRG9uYWh1ZTwvQXV0 aG9yPjxZZWFyPjIw MTI8L111YXI+PFJ1Y051bT43PC9SZWNOdW0+PERpc3BsYX1UZXhOPjxzdHlsZSBm YWNIPSJzdXBI cnNjcmlwdCI+MTM8L3N0eWxlPjwvRGlzcGxheVRleHQ+PHJIY29yZD48cmVjLW51 bWJlcj43PC9y ZWMtbnVtYmVyPjxmb3JlaWduLWtleXM+PGtleSBhcHA9IkVOIiBkYi1 pZDOid2R2Z DJ4MncOMmQ5 eDNleHhhbng1ZZHBlcmR1ZHBOdGEyeGZ4Ij43PC9rZXk+PC9mb3JlaWduLWtleXM+P HJIZilOeXBI IG5hbWU9IkpvdXJuYWwgQXJOaWNsZSI+MTc8L3JIZilOeXBIPjxjb250cmlidXRvcn M+PGF1 dGhv cnM+PGF1dGhvcj5JYWNvYnV6aW8tRG9uYWh1ZSwgQy4gQS48L2F1dGhvcj48YX V0aG9yPlZlbGN1 bGVzY3UsIFYuIEUuPC9hdXRob3I+PGF1dGhvcj5Xb2xmZ2FuZywgQy4gTC48L2F1d Ghvcj48YXV0 aG9yPkhydWJhbiwgUi4gSC48L2F1dGhvcj48L2F1dGhvcnM+PC9jb250cmlidXRvcnM+PGF1 dGgt YWRkcmVzcz5EZXBhcnRtZW50IG9mIFBhdGhvbG9neSwgSm9obnMgSG9wa2lucyB Vbml2ZXJzaXR5 IFNjaG9vbCBvZiBNZWRpY2luZSwgQmFsdGItb3JILCBNRCwgVVNBLjwvYXVOaC 1hZGRyZXNzPjx0 aXRsZXM+PHRpdGxlPkdlbmVOaWMgYmFzaXMgb2YgcGFuY3JIYXMgY2FuY2VyI GR1dmVsb3BtZW50 IGFuZCBwcm9ncmVzc2lvbjogaW5zaWdodHMgZnJvbSB3aG9sZSIleG9tZSBhbmQgd 2hvbGUtZ2Vu b21llHiNlcXVlbmNpbmc8L3RpdGxlPjxzZWNvbmRhcnktdGl0bGU+Q2xpbiBDYW5jZ XIgUmVzPC9z ZWNvbmRhcnktdGl0bGU+PGFsdC1OaXRsZT5DbGluaWNhbCBjYW5jZXIgcmVzZW FyY2ggOiBhbiBv ZmZpY2lhbCBqb3VybmFsIG9mIHRoZ SBBbWVyaWNhbiBBc3NvY21hdGlvbiBmb3Ig Q2FuY2VyIFJI c2VhcmNoPC9hbHQtdGl0bGU+PC90aXRsZXM+PHBlcmlvZGljYWw+PGZ1bGwtdGl ObGU+Q2xpbiBD YW5jZXIgUmVzPC9mdWxsLXRpdGxlPjxhYmJyLTE+Q2xpbmljYWwgY2FuY2VyIH Jlc2VhcmNoIDog YW4gb2ZmaWNpYWwgam91cm5hbCBvZiBOaGUgQW1lcmljYW4gQXNzb2NpYXR pb24gZm9yIENhbmN1 ciBSZXNYXJjaDwvYWJici0xPjwvcGVyaW9kaWNhbD48YWx0LXBlcmlvZGljYWw+PGZ1bGwtdGl0 bGU+Q2xpbiBDYW5jZXIgUmVzPC9mdWxsLXRpdGxlPjxhYmJyLTE+Q2xpbmljYW wgY2FuY2VyIHJI c2VhcmNoIDogYW4gb2ZmaWNpYWwgam91cm5hbCBvZiBOaGUgQWllcmljYW4g QXNzb2NpYXRpb24g Zm9yIENhbmNlciBSZXN1YXJjaDwvYWJiciOxPjwvYWx0LXBlcmlvZGljYWw+PHBh Z2VzPjQyNTct NjU8L3BhZ2VzPjx2b2x1bWU+MTg8L3ZvbHVtZT48bnVtYmVyPjE2PC9udW liZXI+PGtleXdvcmRz PjxrZX13b3JkPkNhcmNpbm9tYSwgUGFuY3JIYXRpYyBEdWN0YWwvZ2VuZXRpY 3MvcGF0aG9sb2d5 PC9rZX13b3JkPjxrZX13b3JkPkRpc2Vhc2UgUHJvZ3Jlc3Npb248L2tleXdvcmQ+PGtleX dvcmQ+RmFtaWx5PC9rZX13b3JkPjxrZX13b3JkPkdlbm9tZSwgSHVtYW48L2tleXdvcmQ+PGt1 eXdvcmQ+R2VybS1MaW51IE11dGF0aW9uPC9rZX13b3JkPjxrZX13b3JkPkhlbWFuczwva2V5d29 yZD48a2V5 d29yZD5OZW9wbGFzbSBJbnZhc212ZW5lc3M8L2tleXdvcmQ+PGtleXdvcmQ+TmVv cGxhc20gTWV0 YXNOYXNpczwva2V5d29yZD48a2V5d29yZD5QYW5jcmVhdGljIE5lb3BsYXNtcy8q Z2VuZXRpY3Mv KnBhdGhvbG9neTwva2V5d29yZD48a2V5d29yZD5TZXF1ZW5jZSBBbmFseXNpcyw gRE5BPC9rZX13 b3JkPjwva2V5d29yZHM+PGRhdGVzPjx5ZWFyPjIwMTI8L311YXI+PHB1Yi1kYXRlc z48ZGF0ZT5B dWcgMTU8L2RhdGU+PC9wdWItZGF0ZXM+PC9kYXRlcz48aXNibj4xMDc4LTAOM zIgKFByaW50KSYj eEQ7MTA30COwNDMyIChMaW5raW5nKTwvaXNibj48YWNjZXNzaW9uLW51bT4 yMjg5NjY5MjwvYWNj ZXNzaW9uLW51bT48dXJscz48cmVsYXR1ZC11cmxzPjxl cmw+aHROcDovL3d3dy5u Y2JpLm5sbS5u aWguZ292L3B1Ym1lZC8yMjg5NjY5MjwvdXJsPjwvcmVsYXR1ZC11cmxzPjwvdXJsc z48Y3VzdG9t Mj4zNDIyNzcxPC9jdXN0b20yPjxlbGVjdHJvbmljLXJlc291cmNlLW51bT4xMC4xMT U4LzEwNzgt MDQzMi5DQ1ItMTItMDMxNTwvZWxlY3Ryb25pYy lyZXNvdXJjZSludWO+PC9yZ WNvcmQ+PC9DaXR1 PjwvRW5kTm90ZT4A 13.

Through these analyses we were able to identify somatic alterations with potentially actionable consequences in 98 of the 101 patients (97%) (Table 1, Supplementary Table 10). Even if one excludes clinical trials related to alterations in KRAS and TP53, over a third (38%) of patients had clinically actionable mutations (Table 1). These alterations included amplification of the HER-2/neu tyrosine kinase ERBB2, the serine and threonine kinases AKT1 and AKT2 genes, the cyclin dependent kinase CDK4 gene, and the E3 ubiquitin ligaseMDM2 gene. We also observed nonsynonymous somatic mutations in the catalytic domains of the phosphatidylinositol-4,5-bisphosphate 3-kinase, PIK3CA, and the v-kit Hardy-Zuckerman 4 feline sarcoma viral oncogene homolog, KIT (Supplementary Table 4). These alterations were at or nearby previously identified somatic mutations in other human cancersPEVuZE5vdGU+PENpdGU+PEF1dGhvcj5Gb3JiZXMLO8LOF1dGhvcj48WWVhcj 4yMDExPC9ZZWFyPjxS ZWNOdW0+OTwvUmVjTnVtPjxEaXNwbGF5VGV4dD48c3R5bGUgZmFjZT0ic3Vw ZXJzY3JpcHQiPjEO PC9zdHlsZT48L0Rpc3BsYX1UZXhOPjxyZWNvcmQ+PHJlYy1udW1iZXI+OTwvcmVj LW5lbWJlcj48 Zm9yZWlnbi1rZXlzPjxrZXkgYXBwPSJFTiIgZGItaWQ9IndkdmQyeDJ3NDJkOXgzZX h4YW54NWRw ZXJkZWRwdHRhMnhmeCI+OTwva2V5PjwvZm9yZWlnbilrZXlzPjxyZWYtdHlwZSB uYWllPSJKb3Vy bmFsIEFydGljbGUiPjE3PC9yZWYtdHlwZT48Y29udHJpYnVOb3JzPjxhdXRob3JzPjxh dXRob3I+Rm9yYmVzLCBTLiBBLjwvYXV0aG9yPjxhdXRob3I+QmluZGFsLCBOLjwvYXVOa G9yPjxhdXRob3I+QmFtZm9yZCwgUy48L2F1dGhvcj48YXV0aG9yPkNvbGUsIEMuPC9hdXRob3I+PGF 1dGhvcj5Lb2ss IEMuIFkuPC9hdXRob3I+PGF1dGhvcj5CZWFyZSwgRC48L2F1dGhvcj48YXV0aG9y PkppYSwgTS48 L2F1dGhvcj48YXV0aG9yPlNoZXBoZXJkLCBSLjwvYXV0aG9yPjxhdXRob3I+TGV1 bmcslEsuPC9h dXRob3I+PGF1dGhvcj5NZW56aWVzLCBBLjwvYXV0aG9yPjxhdXRob3I+VGVhZ3 VlLCBKLiBXLjwv YXV0aG9yPjxhdXRob3I+Q2FtcGJlbGwsIFAuIEouPC9hdXRob3I+PGF1dGhvcj5TdHJ hdHRvbiwg TS4gUi48L2F1dGhvcj48YXV0aG9yPkZ1dHJlYWwsIFAuIEEuPC9hdXRob3I+PC9 hd XRob3JzPjwv Y29udHJpYnVOb3JzPjxhdXRoLWFkZHJlc3M+Q2FuY2VyIEdlbm9tZSBQcm9qZWN0 LCBXZWxsY29t ZSBUcnVzdCBTYW5nZXIgSW5zdG1OdXR1LCBXZWxsY29tZSBUcnVzdCBHZW5vb WUgQ2FtcHVzLCBI aW54dG9uLCBDQjEwIDFTQ SBDYW1icmlkZ2UsIFVLLjwvYXVOaC1hZGRyZXNzP jxOaXRsZXM+PHRp dGx1PkNPU01JQzogbWluaW5nIGNvbXBsZXR1IGNhbmNlciBnZW5vbWVzIGluIHRo ZSBDYXRhbG9n dWUgb2YgU29tYXRpYyBNdXRhdGlvbnMgaW4gQ2FuY2VyPC90aXRsZT48c2Vjb2 5kYXJ5LXRpdGxl Pk51Y2xlaWMgQWNpZHMgUmVzPC9zZWNvbmRhcnktdGl0bGU+PGFsdC1OaXRsZ T50dWNsZWIjIGFJ aWRzIHJlc2VhcmNoPC9hbHQtdGl0bGU+PC90aXRsZXM+PHBlcmlvZGljYWw+PGZ lbGwtdGl0bGU+TnVjbGVpYyBBY21kcyBSZXM8L2Z1bGwtdGl0bGU+PGFiYnItMT50dWNsZWljIGF jaWRzIHJlc2Vh cmNoPC9hYmJyLTE+PC9wZXJpb2RpY2FsPjxhbHQtcGVyaW9kaWNhbD48ZnVsbC 1OaXRsZT5OdWNs ZWljIEFjaWRzIFJlczwvZnVsbC1OaXRsZT48YWJici0xPk51Y2xlaWMgYWNpZHMgc mVzZWFyY2g8 L2FiYnItMT48L2FsdC1 wZXJpb2RpY2FsPjxwYWdlcz5EOTQ1LTUwPC9wYWdlcz48 dm9sdWllPjM5 PC92b2x1bWU+PG51bWJlcj5EYXRhYmFzZSBpc3N1ZTwvbnVtYmVyPjxrZX13b3Jk cz48a2V5d29y ZD5DZWxsIExpbmUsIFR1bW9yPC9rZX13b3JkPjxrZX13b3JkPkRhdGEgTW1uaW5nP C9rZX13b3Jk PjxrZX13b3JkPipEYXRhYmFzZXMsIE51Y2xlaWMgQWNpZDwva2V5d29yZD48a2V 5d29yZD4qR2Vu b211LCBIdW1hbjwva2V5d29yZD48a2V5d29yZD5IdW1hbnM8L2tleXdvcmQ+PGtleX dvcmQ+Kkl 1 dGF0aW9uPC9rZX13b3JkPjxrZX13b3JkPk5lb3BsYXNtcy8qZ2VuZXRpY3M8L2tleXdv cmQ+PGtl eXdvcmQ+VXNlci1Db21wdXRlciBJbnRlcmZhY2U8L2tleXdvcmQ+PC9rZX13b3Jkcz4 8ZGF0ZXM+PHIIYXI+MjAxMTwveWVhcj48cHViLWRhdGVzPjxkYXRlPkphbjwvZGF0ZT48L3B lYilkYXRlcz48 L2RhdGVzPjxpc2JuPjEzNjItNDk2MiAoRWxIY3Ryb25pYykmI3hEOzAzMDUtMTAO OCAoTGlua2lu Zyk8L2lzYm4+PGFjY2Vzc2lvbiludWO+MjA5NTI0MDU8L2FjY2Vzc2lvbiludWO+P HVybHM+PHJI bGFOZWQtdXJscz48dXJsPmhOdHA6Ly93d3cubmNiaS5ubGOubmloLmdvdi9wdWJtZ WQvMjA5NTI0 MDU8L3VybD48L3JIbGFOZWQtdXJscz48L3VybHM+PGNlc3RvbTI+MzAxMzc4NT wvY3VzdG9tMj48 ZWxlY3Ryb25pYy lyZXNvdXJjZSludWO+MTAuMTA5My9uYXIvZ2txOTI5PC9lbG VjdHJvbmljLXJl c291cmNILW51bT48L3JIY29yZD48LONpdGU+PC9FbmROb3R1PgB=PEVuZE5vdGU+PENpdGU+PEF1dGhvcj5Gb3JiZXM8LOF1dGhvcj48WWVhcj4yMDE xPC9ZZWFyPjxS ZWNOdW0+OTwvUmVj TnVtPjxEaXNwbGF5VGV4dD48c3R5bGUgZmFjZT0ic3Vw ZXJzY3JpcHQiPjEO PC9zdHlsZT48L0Rpc3BsYX1UZXhOPjxyZWNvcmQ+PHJIYy ludW1iZXI+OTwvcmVj LW5lbWJlcj48 Zm9yZWlnbi1rZXlzPjxrZXkgYXBwPSJFTiIgZGItaWQ9IndkdmQyeDJ3NDJkOXgzZX h4YW54NWRw ZXJkZWRwdHRhMnhmeCI+OTwva2V5PjwvZm9yZWlnbi1rZXlzPjxyZWYtdH1wZSB uYW11PSJKb3Vy bmFsIEFydGljbGUiPjE3PC9yZWYtdH1wZT48Y29udHJpYnVOb3JzPjxhdXRob3JzPjxh dXRob3I+Rm9yYmVzLCBTLiBBLjwvYXV0aG9yPjxhdXRob3I+QmluZGFsLCBOLjwvYXVOa G9yPjxhdXRob3I+QmFtZm9yZCwgUy48L2F1dGhvcj48YXV0aG9yPkNvbGUsIEMuPC9hdXRob3I+PGF 1 dGhvcj5Lb2ss IEMuIFkuPC9hdXRob3I+PGF1dGhvcj5CZWFyZSwgRC48L2F1dGhvcj48YXV0aG9y PkppYSwgTS48 L2F1dGhvcj48YXV0aG9yPINoZXBoZXJkLCBSLjwvYXV0aG9yPjxhdXRob3I+TGV1 bmcsIEsuPC9h dXRob3I+PGF1dGhvcj5NZW56aWVzLCBBLjwvYXV0aG9yPjxhdXRob3I+VGVhZ3 VILCBKLiBXLjwv YXV0aG9yPjxhdXRob3I+Q2FtcGJlbGwsIFAuIEouPC9hdXRob3I+PGF1dGhvcj5TdHJ hdHRvbiwg TS4gUi48L2F1dGhvcj48YXV0aG9yPkZ1dHJIYWwsIFAuIEEuPC9hdXRob3I+PC9 hd XRob3JzPjwv Y29udHJpYnVOb3JzPjxhdXRoLWFkZHJlc3M+Q2FuY2VyIEdlbm9tZ SBQcm9qZWN0 LCBXZWxsY29t ZSBUcnVzdCBTYW5nZXIgSW5zdG1OdXRILCBXZWxsY29tZSBUcnVzdCBHZW5vb WUgQ2FtcHVzLCBI aW54dG9uLCBDQjEwIDFTQ SBDYW1icmlkZ2UsIFVLLjwvYXVOaC1hZGRyZXNzP jxOaXRsZXM+PHRp dGxlPkNPU01JQzogbWluaW5nIGNvbXBsZXRlIGNhbmNlciBnZW5vbWVzIGluIHRo ZSBDYXRhbG9n dWUgb2YgU29tYXRpYyBNdXRhdGlvbnMgaW4gQ2FuY2VyPC90aXRsZT48c2Vjb2 5kYXJ5LXRpdGxl Pk51Y2xlaWMgQWNpZHMgUmVzPC9zZWNvbmRhcnktdGl0bGU+PGFsdC1OaXRsZ T50dWNsZWIjIGFJ aWRzIHJlc2VhcmNoPC9hbHQtdGl0bGU+PC90aXRsZXM+PHBlcmlvZGljYWw+PGZ lbGwtdGl0bGU+TnVjbGVpYyBBY21kcyBSZXM8L2Z1bGwtdGl0bGU+PGFiYnItMT50dWNsZWljIGF jaWRzIHJlc2Vh cmNoPC9hYmJyLTE+PC9wZXJpb2RpY2FsPjxhbHQtcGVyaW9kaWNhbD48ZnVsbC 1OaXRsZT5OdWNs ZWljIEFjaWRzIFJlczwvZnVsbC1OaXRsZT48YWJici0xPk51Y2xlaWMgYWNpZHMgc mVzZWFyY2g8 L2FiYnItMT48L2FsdC1 wZXJpb2RpY2FsPjxwYWdlcz5EOTQ1LTUwPC9wYWdlcz48 dm9sdWllPjM5 PC92b2x1bWU+PG51bWJlcj5EYXRhYmFzZSBpc3N1ZTwvbnVtYmVyPjxrZX13b3Jk cz48a2V5d29y ZD5DZWxsIExpbmUsIFR1bW9yPC9rZX13b3JkPjxrZX13b3JkPkRhdGEgTWluaW5nP C9rZX13b3Jk PjxrZX13b3JkPipEYXRhYmFzZXMsIE51Y2xlaWMgQWNpZDwva2V5d29yZD48a2V 5d29yZD4qR2Vu b211LCBIdW1hbjwva2V5d29yZD48a2V5d29yZD5IdW1hbnM8L2tleXdvcmQ+PGtleX dvcmQ+Kkl 1 dGF0aW9uPC9rZX13b3JkPjxrZX13b3JkPk5lb3BsYXNtcy8qZ2VuZXRpY3M8L2tleXdv cmQ+PGtl eXdvcmQ+VXNlci1Db21wdXRlciBJbnRlcmZhY2U8L2tleXdvcmQ+PC9rZX13b3Jkcz4 8ZGF0ZXM+PHllYXI+MjAxMTwveWVhcj48cHViLWRhdGVzPjxkYXRlPkphbjwvZGF0ZT48L3B 1YilkYXRlcz48 L2RhdGVzPjxpc2JuPjEzNjItNDk2MiAoRWxlY3Ryb25pYykmI3hEOzAzMDUtMTAO OCAoTGlua2lu Zyk8L2lzYm4+PGFjY2Vzc2lvbiludWO+MjA5NTI0MDU8L2FjY2Vzc2lvbiludWO+P HVybHM+PHJl bGFOZWQtdXJscz48dXJsPmhOdHA6Ly93d3cubmNiaS5ubGOubmloLmdvdi9wdWJtZ WQvMjA5NTI0 MDU8L3VybD48L3JlbGFOZWQtdXJscz48L3VybHM+PGNlc3RvbTI+MzAxMzc4NT wvY3VzdG9tMj48 ZWxlY3Ryb25pYy lyZXNvdXJjZSludWO+MTAuMTA5My9uYXIvZ2txOTI5PC9lbG VjdHJvbmljLXJl c291cmNlLW51bT48L3J1Y29yZD48LONpdGU+PC9FbmROb3R1PgB=14. In addition, we identified three patients with truncating somatic alterations in BRCA2: two with heterozygous somatic nonsense alterations, a patient with a frameshift alteration, and a fourth patient with a germline frameshift in BRCA2 together with a loss of heterozygosity (LOH) in the matched-tumor sample (Supplementary Table 4).

These alterations represent potential targets of clinical intervention in pancreatic cancer. Poly(adenosine diphosphate [ADP]-ribose) polymerase (PARP) inhibitors and DNA damaging agents such as cisplatin and mitomycin C have been shown to provide a synthetic lethal therapeutic strategy for treatment of cancers with defects in components of the homologous recombination repair pathway, such as BRCA1/2PEVuZE5vdGU+PENpdGU+PEF1dGhvcj5Gb2dlbG1hbjwvQXV0aG9yPjxZZ WFyPjIwMTE8L111YXI+PFJIY051bT4yMTwvUmVjTnVtPjxEaXNwbGF5VGV4dD48c3R5bGUgZmFjZT0ic3V wZXJzY3JpcHQi PjE1LTE4PC9zdHlsZT48L0Rpc3BsYX1UZXhOPjxyZWNvcmQ+PHJlYyludWliZXI+MjE8L3JlYylu dW1iZXI+PGZvcmVpZ24ta2V5cz48a2V5IGFwcDOiRU4iIGRiLWlkPSJ3ZHZkMngydz QyZD14M2V4 eGFueDVkcGVyZGVkcHROYTJ4ZngiPjIxPC9rZXk+PC9mb3JlaWduLWtleXM+PHJlZ ilOeXBlIG5h bWU9IkpvdXJuYWwgQXJOaWNsZSI+MTc8L3JlZilOeXBlPjxjb250cmlidXRvcnM+P GF1dGhvcnM+PGF1dGhvcj5Gb2dlbG1hbiwgRC4gUi48L2F1dGhvcj48YXV0aG9yPldvbGZmLCBSLi BBLjwvYXVO aG9yPjxhdXRob3I+S29wZXR6LCBTLjwvYXV0aG9yPjxhdXRob3I+SmF2bGUsIEOuP C9hdXRob3I+PGF1dGhvcj5CcmFkbGV5LCBDLjwvYXV0aG9yPjxhdXRob3I+TW9rLCBJLjwvYXV 0aG9yPjxhdXRo b3I+Q2FiYW5pbGxhcywgRi48L2F1dGhvcj48YXV0aG9yPkFiYnJ1enplc2UsIEouIEwu PC9hdXRo b3I+PC9hdXRob3JzPjwvY29udHJpYnVOb3JzPjxhdXRoLWFkZHJlc3M+TS5ELiBBb mRlcnNvbiBD YW5jZXIgQ2VudGVyLCBIb3VzdG9uLCBUWCA3NzAzMCwgVVNBLiBkZm9nZW xtYW5AbWRhbmRlcnNv bi5vcmc8L2F1dGgtYWRkcmVzcz48dGl0bGVzPjx0aXRsZT5FdmlkZW5jZSBmb3IgdG hlIGVmZmlj YWN5IG9mIEluaXBhcmliLCBhIFBBUlAtMSBpbmhpYm10b3IsIGluIEJSQOEyLWFzc 29jaWFOZWQg cGFuY3J1YXRpYyBjYW5jZXI8L3RpdGxlPjxzZWNvbmRhcnktdGl0bGU+QW50aWN hbmNlciBSZXM8 L3N1Y29uZGFyeSOaXRsZT48YWx0LXRpdGxlPkFudGljYW5jZXIgcmVzZWFyY2g 8L2FsdC1OaXRs ZT48L3RpdGxlcz48cGVyaW9kaWNhbD48ZnVsbC1OaXRsZT5BbnRpY2FuY2VyIFJlc zwvZnVsbC10 aXRsZT48YWJiciOxPkFudGljYW5jZXIgcmVzZWFyY2g8L2FiYnItMT48L3B1cmlvZG ljYWw+PGFs dC1wZXJpb2RpY2FsPjxmdWxsLXRpdGxlPkFudGljYW5jZXIgUmVzPC9mdWxsLXR pdGxlPjxhYmJy LTE+QW50aWNhbmNlciByZXN1YXJjaDwvYWJici0xPjwvYWx0LXBlcmlvZGljYW w+PHBhZ2VzPjE0 MTctMjA8L3BhZ2VzPjx2b2x1bWU+MzE8L3ZvbHVtZT48bnVtYmVyPjQ8L251bWJ1 cj48a2V5d29y ZHM+PGtleXdvcmQ+QWRlbm9jYXJjaW5vbWEvZ2VuZXRpY3MvKnN1Y29uZGFye S90aGVyYXB5PC9r ZX13b3JkPjxrZX13b3JkPkJSQOEyIFByb3RlaW4vKmdlbmV0aWNzPC9rZX13b3JkPjxr ZX13b3Jk PkJlbnphbWlkZXMvKnRoZXJhcGV1 dGljIHVzZTwva2V5d29yZD48a2V5d29yZD5Cc mVhc3QgTmVv cGxhc21zL2dlbmV0aWNzLypwYXRob2xvZ3kvdGhlcmFweTwva2V5d29yZD48a2V5d 29yZD5GZW1h bGU8L2tleXdvcmQ+PGtleXdvcmQ+R2VybS1MaW51IE11dGF0aW9uL2dlbmV0aWNz PC9rZX13b3Jk PjxrZX13b3JkPkhlbWFuczwva2V5d29yZD48a2V5d29yZD5NaWRkbGUgQWdIZDwva 2V5d29yZD48 a2V5d29yZD50ZW9wbGFzb SBSZWN1cnJlbmN1LCBMb2NhbC9nZW5ldGljcy8qcGF 0aG9sb2d5L3Ro ZXJhcHk8L2tleXdvcmQ+PGtleXdvcmQ+UGFuY3J1YXRpYyBOZW9wbGFzbXMvZ2 VuZXRpY3MvKnNI Y29uZGFyeS90aGVyYXB5PC9rZX13b3JkPjxrZX13b3JkP1BvbHkoQURQLXJpYm9zZ SkgUG9seWl cmFzZXMvKmFudGFnb25pc3RzICZhbXA7IGluaGliaXRvcnM8L2tleXdvcmQ+PGtleX dvcmQ+VHJI YXRtZW50IE91dGNvbWU8L2tleXdvcmQ+PC9rZX13b3Jkcz48ZGF0ZXM+PHIIYXI+MjAxMTwveWVh cj48cHViLWRhdGVzPjxkYXRlPkFwcjwvZGF0ZT48L3B1Yi1kYXR1cz48L2RhdGVzP jxpc2JuPjE3 OTEtNzUzMCAoRWx1Y3Ryb25pYykmI3hEOzAyNTAtNzAwNSAoTGlua2luZyk8L21 zYm4+PGFjY2Vz c2lvbiludWO+MjElMDgzOTU8L2FjY2Vzc2lvbiudWO+PHVybHM+PHJ1bGFOZWQt dXJscz48dXJs PmhOdHA6Ly93d3cubmNiaS5ubGOubmloLmdvdi9wdWJtZWQvMjElMDgzOTU8L3V ybD48L3J1bGF0 ZWQtdXJscz48L3VybHM+PC9yZWNvcmQ+PC9DaXR1PjxDaXRIPjxBdXRob3I+Rm9 uZzwvQXV0aG9y PjxZZWFyPjIwMDk8L 11lYXI+PFJ1Y051bT4xMDwvUmVj TnVtPjxyZWNvcmQ+PHJI YyludW liZXI+MTA8L3JIYyludW liZXI+PGZvcmVpZ24ta2V5cz48a2V5IGFwcDOiRU4iIGRiLW1kP SJ3ZHZkMngy dzQyZD14M2V4eGFueDVkcGVyZGVkcHROYTJ4ZngiPjEwPC9rZXk+PC9mb3JlaWd uLWtleXM+PHJI Zi1OeXBlIG5hbWU9IkpvdXJuYWwgQXJOaWNsZSI+MTc8L3JZiOeXBIPjxjb250cml idXRvcnM+PGF1dGhvcnM+PGF1dGhvcj5Gb25nLCBQLiBDLjwvYXV0aG9yPjxhdXRob3I+Qm9z cywgRC4gUy48 L2F1dGhvcj48YXV0aG9yPllhcCwgVC4gQS48L2F1dGhvcj48YXV0aG9yP1R1dHQsIE EuPC9hdXRo b3I+PGF1dGhvcj5XdSwgUC48L2F1dGhvcj48YXV0aG9yPk1lcmdl aS1Sb2Vsdmluayw gTS48L2F1 dGhvcj48YXV0aG9yPk1vcnRpbWVyLCBQLjwvYXV0aG9yPjxhdXRob3I+U3dhaXNs YW5kLCBILjwv YXV0aG9yPjxhdXRob3I+TGF1LCBBLjwvYXV0aG9yPjxhdXRob3I+TyZhcG9zOONv bm5vciwgTS4g Si48L2F1dGhvcj48YXV0aG9yPkFzaHdvcnRoLCBBLjwvYXV0aG9yPjxhdXRob3I+Q 2FybWljaGF1 bCwgSi48L2F1dGhvcj48YXV0aG9yPktheWUsIFMuIEIuPC9hdXRob3I+PGF1dGhvcj5 TY2hlbGxl bnMsIEouIEguPC9hdXRob3I+PGF1dGhvcj5kZSBCb25vLCBKLiBTLjwvYXV0aG9yP jwvYXV0aG9y cz48L2NvbnRyaWJ1dG9ycz48YXVOaC1hZGRyZXNzPkRydWcgRGV2ZWxvcGllbnQ gVW5pdCwgUm95 YWwgTWFyc2RlbiBOYXRpb25hbCBIZWFsdGggU2VydmljZ SBGb3VuZGFOaW9uIF RydXNOIGFuZCB0 aGUgSW5zdG1OdXR1IG9mIENhbmNlciBSZXNYXJjaCwgU3VOdG9uLCBTdXJyZXk sIFVuaXR1ZCBL aW5nZG9tLjwvYXVOaC1hZGRyZXNzPjx0aXRsZXM+PHRpdGxlPkluaGliaXRpb24g b2YgcG9seShB RFAtcmlib3N1KSBwb2x5bWVyYXN1IGluIHR1bW9ycyBmcm9tIEJSQOEgbXV0YXRp b24gY2Fycmll cnM8L3RpdGxlPjxzZWNvbmRhcnktdGl0bGU+TiBFbmdsIEogTWVkPC9zZWNvbmR hcnktdGl0bGU+PGFsdC1OaXRsZT5UaGUgTmV3IEVuZ2xhbmQgam91cm5hbCBvZiBtZWRpY2luZT wvYWx0LXRpdGxl PjwvdGl0bGVzPjxwZXJpb2RpY2FsPjxmdWxsLXRpdGxlPk4gRW5nbCBKIE llZDwv ZnVsbC1OaXRs ZT48YWJici0xPlRoZSBOZXcgRW5nbGFuZCBqb3VybmFsIG9mIG11ZGljaW51PC9h YmJyLTE+PC9w ZXJpb2RpY2FsPjxhbHQtcGVyaW9kaWNhbD48ZnVsbC1OaXRsZT50IEVuZ2wgSiBN ZWQ8L2Z1bGwt dG10bGU+PGFiYnItMT5UaGUgTmV3IEVuZ2xhbmQgam91cm5hbCBvZiBtZWRpY21 uZTwvYWJiciOx PjwvYWx0LXBlcmlvZGljYWw+PHBhZ2VzPjEyMyOzNDwvcGFnZXM+PHZvbHVtZ T4zNjE8L3ZvbHVt ZT48bnVtYmVyPjI8L251bWJlcj48a2V5d29yZHM+PGtleXdvcmQ+QWR1bHQ8L2tle XdvcmQ+PGtl eXdvcmQ+QWd1ZDwva2V5d29yZD48a2V5d29yZD5BZ2VkLCA4MCBhbmQgb3Zlcj wva2V5d29yZD48 a2V5d29yZD5Eb3N1LVJlc3BvbnNlIFJlbGFOaW9uc2hpcCwgRHJ1Zzwva2V5d29yZD4 8a2V5d29y ZD5EcnVnIERlc2lnbjwva2V5d29yZD48a2V5d29yZD5GZW1hbGU8L2tleXdvcmQ+P GtleXdvcmQ+KkdlbmVzLCBCUkNBMTwva2V5d29yZD48a2V5d29yZD4qR2VuZXMsIEJSQOEyPC 9rZX13b3JkPjxr ZX13b3JkPkdlcmOtTGluZSBNdXRhdGlvbjwva2V5d29yZD48a2V5d29yZD5IZXRlcm9 6eWdvdGU8 L2tleXdvcmQ+PGtleXdvcmQ+SHVtYW5zPC9rZX13b3JkPjxrZX13b3JkPk1hbGU8L2tl eXdvcmQ+PGtleXdvcmQ+TW1kZGx1IEFnZWQ8L2tleXdvcmQ+PGtleXdvcmQ+TmVvcGxhc21zL ypkcnVnIHRo ZXJhcHkvZ2VuZXRpY3M8L2tleXdvcmQ+PGtleXdvcmQ+UGh0aGFsYXppbmVzL2F kdmVyc2UgZWZm ZWNOcy9waGFybWFjb2tpbmV0aWNzLypOaGVyYXBldXRpYyB1c2U8L2tleXdvcmQ+PGtleXdvcmQ+UG1wZXJhemluZXMvYWR2ZXJzZ SB1ZmZ1Y3RzL3BoYXJtYWNva2luZXRpY3MvK nRoZXJhcGV 1dGlj IHVzZTwva2V5d29yZD48a2V5d29yZD5Qb2x5KEFEUC1yaWJvc2UpIFBvbHltZXJhc 2VzLyphbnRh Z29uaXNOcyAmYW1wOyBpbmhpYm10b3JzPC9rZX13b3JkPjxrZX13b3JkP11vdW5nIEF kdWxOPC9r ZX13b3JkPjwva2V5d29yZHM+PGRhdGVzPjx5ZWFyPjIwMDk8L311YXI+PHB1Yi1kY XR1cz48ZGF0 ZT5KdWwgOTwvZGF0ZT48L3B1Yi1kYXRlcz48L2RhdGVzPjxpc2JuPjElMzMtNDQ wNiAoRWxlY3Ry b25pYykmI3hEOzAwMjgtNDc5MyAoTGlua2luZyk8L2lzYm4+PGFjY2Vzc2lvbiludW 0+MTklNTM2 NDE8L2FjY2Vzc2lvbiludWO+PHVybHM+PHJ1bGFOZWQtdXJscz48dXJsPmhOdHA6 Ly93d3cubmNi aS5ubGOubmloLmdvdi9wdWJtZWQvMTk1NTM2NDE8L3VybD48L3J1bGFOZWQtdX Jscz48L3VybHM+PGVsZWN0cm9uaWMtcmVzb3VyY2UtbnVtPjEwLjEwNTYvTkVKTW9hMDkwMDI xMjwvZWxlY3Ryb25p Yy 1yZXNvdXJjZS1udWO+PC9yZWNvcmQ+PC9DaXR1PjxDaXR1PjxBdXRob3I+dmF uIGR1ciBIZWlq ZGVuPC9BdXRob3I+PFIIYXI+MjAwNTwvWWVhcj48UmVjTnVtPjE3PC9SZWNOd WO+PHJ1Y29yZD48 cmVjLW51bWJlcj4xNzwvcmVjLW51bWJlcj48Zm9yZWlnbi1rZXlzPjxrZXkgYXBwPS JFTiIgZGIt aWQ9IndkdmQyeDJ3NDJkOXgzZXh4YW54NWRwZXJkZWRwdHRhMnhmeCI+MTc 8L2tleT48L2ZvcmVp Z24ta2V5cz48cmVmLXR5cGUgbmFtZT0iSm91cm5hbCBBcnRpY2xlIj4xNzwvcmVm LXR5cGU+PGNv bnRyaWJ1dG9ycz48YXV0aG9ycz48YXV0aG9yPnZhbiBkZXIgSGVpamRlbiwgT S4gU y48L2F1dGhv cj48YXV0aG9yPkJyb2R5LCBKLiBSLjwvYXV0aG9yPjxhdXRob3I+RGV6ZW50amU sIEQuIEEuPC9h dXRob3I+PGF1dGhvcj5HYWxsbWVpZXIsIEUuPC9hdXRob3I+PGF1dGhvcj5DdW5u aW5naGFtLCBT LiBDLjwvYXV0aG9yPjxhdXRob3I+U3dhcnR6LCBNLiBKLjwvYXV0aG9yPjxhdXRo b3I+RGVNYXJ6 bywgQS4gTS48L2F1dGhvcj48YXV0aG9yPk9mZmVyaGF 1 cywgRy4gSi48L2F1dGhvc j48YXV0aG9y PklzYWNvZmYsIFcuIEguPC9hdXRob3I+PGF1dGhvcj5IcnViYW4sIFIuIEguPC9hdXR ob3I+PGF1 dGhvcj5LZXJuLCBTLiBFLjwvYXV0aG9yPjwvYXV0aG9ycz48L2NvbnRyaWJ1dG9y cz48YXVOaClh ZGRyZXNzPkRlcGFydG1lbnQgb2YgT25jb2xvZ3ksIFRoZSBTb2wgR29sZG1hbiBQY W5jcmVhdGlj IENhbmNlciBSZXN1YXJjaCBDZW50ZXIsIFRoZSBKb2hucyBIb3BraW5zIFVuaXZlcn NpdHkgU2No b29sIG9mIE11ZG1jaW51LCBCYWxOaW1vemUslE1henlsYW5kIDIxMjMxLCBVUOEu PC9hdXRoLWFk ZHJlc3M+PHRpdGxlcz48dGl0bGU+SW4gdm12byB0aGVyYXBldXRpYyByZXNwb25 zZXMgY29udGlu Z2VudCBvbiBGYW5jb25pIGFuZW1pYS9CUkNBMiBzdGFOdXMgb2YgdGhlIHR1b W9yPC9OaXRsZT48 c2Vjb25kYXJ5LXRpdGxlPkNsaW4gQ2FuY2VyIFJlczwvc2Vjb25kYXJ5LXRpdGxlPjx hbHQtdGIO bGU+Q2xpbmljYWwgY2FuY2VyIHJlc2VhcmNoIDogYW4gb2ZmaWNpYWwgam91c m5hbCBvZiBOaGUg QW1lcmljYW4gQXNzb2NpYXRpb24gZm9yIENhbmNlciBSZXN1YXJjaDwvYWxOLX RpdGxlPjwvdGIO bGVzPjxwZXJpb2RpY2FsPjxmdWxsLXRpdGxlPkNsaW4gQ2FuY2VyIFJlczwvZnVsb C10aXRsZT48 YWJiciOxPkNsaW5pY2FsIGNhbmNlciByZXN1YXJjaCA6IGFuIG9mZmljaWFsIGpvdX JuYWwgb2Yg dGhlIEFtZXJpY2FuIEFzc29jaWFOaW9uIGZvciBDYW5jZXIgUmVzZWFyY2g8L2FiY nItMT48L3B1 cmlvZGljYWw+PGFsdC1wZXJpb2RpY2FsPjxmdWxsLXRpdGxlPkNsaW4gQ2FuY2V yIFJlczwvZnVs bC1OaXRsZT48YWJiciOxPkNsaW5pY2FsIGNhbmNlciByZXN1YXJjaCA6IGFuIG9mZ mljaWFsIGpv dXJuYWwgb2YgdGhlIEFtZXJpY2FuIEFzc29jaWFOaW9uIGZvciBDYW5jZXIgUmVz ZWFyY2g8L2Fi YnItMT48L2FsdC1wZXJpb2RpY2FsPjxwYWdlcz43NTA4LTE1PC9wYWdlcz48dm9s dWllPjExPC92 b2x1bWU+PG51bWJlcj4yMDwvbnVtYmVyPjxrZX13b3Jkcz48a2V5d29yZD5BbmltY WxzPC9rZX13 b3JkPjxrZX13b3JkPkFudGluZW9wbGFzdGljIEFnZW50cy9waGFybWFjb2xvZ3k8L2tle XdvcmQ+PGtleXdvcmQ+QXBvcHRvc2lzL2RydWcgZWZmZWNOczwva2V5d29yZD48a2V5d29 yZD5CUkNBMiBQ cm90ZWluL2RlZmljaWVuY3kvZ2VuZXRpY3M8L2tleXdvcmQ+PGtleXdvcmQ+Q2Fz cGFzZXMvbWVO YWJvbGlzbTwva2V5d29yZD48a2V5d29yZD5DZWxsIEN5Y2x1L2RydWcgZWZmZW NOczwva2V5d29y ZD48a2V5d29yZD5DZWxsIExpbmUsIFR1bW9yPC9rZX13b3JkPjxrZX13b3JkPkN1bG wgU3Vydml2 YWwvZHJ1ZyB1ZmZ1Y3RzPC9rZX13b3JkPjxrZX13b3JkPkNobG9yYWlidWNpbC9wa GFybWFjb2xv Z3k8L2tleXdvcmQ+PGtleXdvcmQ+Q2lzcGxhdGluL3BoYXJtYWNvbG9neTwva2V5d 29yZD48a2V5 d29yZD5Dcm9zcy1MaW5raW5nIFJlYWdlbnRzLypwaGFybWFjb2xvZ3kvdGhlcmFwZ XVOaWMgdXN1 PC9rZX13b3JkPjxrZX13b3JkPkRlb3h5Y310aWRpbmUvYW5hbG9ncyAmYW1wOyBkZ XJpdmF0aXZ1 cy9waGFybWFjb2xvZ3k8L2tleXdvcmQ+PGtleXdvcmQ+RG9zZS1SZXNwb25zZSBSZ WxhdGlvbnNo aXAsIERydWc8L2tleXdvcmQ+PGtleXdvcmQ+RG94b3J1YmljaW4vcGhhcm1hY29sb2 d5PC9rZX13 b3JkPjxrZX13b3JkPkVOb3Bvc2lkZS9waGFybWFjb2xvZ3k8L2tleXdvcmQ+PGtleXdvc mQ+RmFu Y29uaSBBbmVtaWEgQ29tcGxlbWVudGF0aW9uIEdyb3VwIEMgUHJvdGVpbi9kZW ZpY211bmN5L2dl bmV0aWNzPC9rZX13b3JkPjxrZX13b3JkPkZhbmNvbmkgQW5lbW1hIENvbXBsZWlb nRhdGlvbiBH cm91cCBHIFByb3R1aW4vZGVmaWNpZW5jeS9nZW5ldGljczwva2V5d29yZD48a2V5 d29yZD5GYW5j b25pIEFuZW1pYSBDb21wbGVtZW50YXRpb24gR3JvdXAgUHJvdGVpbnMvZGVma WNpZW5jeS8qZ2Vu ZXRpY3M8L2tleXdvcmQ+PGtleXdvcmQ+RmVtYWx1PC9rZX13b3JkPjxrZX13b3JkPk ZsdW9yb3Vy YWNpbC9waGFybWFjb2xvZ3k8L2tleXdvcmQ+PGtleXdvcmQ+SHVtYW5zPC9rZX13 b3JkPjxrZX13 b3JkPkluaGliaXRvcnkgQ29uY2VudHJhdGlvbiA1MDwva2V5d29yZD48a2V5d29yZD5 NZWxwaGFs YW4vcGhhcm1hY29sb2d5PC9rZX13b3JkPjxrZX13b3JkPk1pY2U8L2tleXdvcmQ+PGtl eXdvcmQ+TWIjZSwgTnVkZTwva2V5d29yZD48a2V5d29yZD5NaXRvbXljaW4vKnBoYXJtYWN vbG9neS90aGVy YXBIdXRpYyB1c2U8L2tleXdvcmQ+PGtleXdvcmQ+TXV0YXRpb248L2tleXdvcmQ+PGtleXdvcmQ+UGFjbG1OYXh1bC9waGFybWFjb2xvZ3k8L2tleXdvcmQ+PGtleXdvcmQ+UGFuY3JlY XRpYyBOZW9w bGFzbXMvKmRydWcgdGhlcmFweS9nZW5ldGljcy9wYXRob2xvZ3k8L2tleXdvcmQ+PGtleXdvcmQ+VG1tZSBGYWNOb3JzPC9rZX13b3JkPjxrZX13b3JkPlZpbmJsYXNOaW51L3BoYXJtYW NvbG9neTwv a2V5d29yZD48a2V5d29yZD5YZW5vZ3JhZnQgTW9kZWwgQW50aXR1bW9yIEFzc2 F5cy9tZXRob2Rz PC9rZX13b3JkPjwva2V5d29yZHM+PGRhdGVzPjx5ZWFyPjIwMDU8L311YXI+PHB1 YilkYXRlcz48 ZGF0ZT5PY3QgMTU8L2RhdGU+PC9wdWItZGF0ZXM+PC9kYXRlcz48aXNibj4xM Dc4LTA0MzIgKFBy aW50KSYjeEQ7MTA3OC0wNDMyIChMaW5raW5nKTwvaXNibj48YWNjZXNzaW9 uLW51bT4xNjIOMzgy NTwvYWNjZXNzaW9uLW51bT48dXJscz48cmVsYXR1ZC11cmxzPjxl cmw+aHROcD ovL3d3dy5uY2Jp Lm5sbS5uaWguZ292L3B1Ym11ZC8xNjIOMzgyNTwvdXJsPjwvcmVsYXR1ZC11cmxz PjwvdXJscz48 ZWxlY3Ryb25pYylyZXNvdXJjZSludWO+MTAuMTE1OC8xMDc4LTAOMzIuQONS LTA1LTEwNDg8L2Vs ZWN0cm9uaWMtcmVzb3VyY2UtbnVtPjwvcmVjb3JkPjwvQ210ZT48Q210ZT48QXV0 aG9yPlZpbGxh cnJvZWw8LOF1dGhvcj48WWVhcj4yMDExPC9ZZWFyPjxSZWNOdW0+MjA8L1J1Y 05lbT48cmVjb3Jk PjxyZWMtbnVtYmVyPjIwPC9yZWMtbnVtYmVyPjxmb3JlaWduLWtleXM+PGtleSBh cHA9IkVOIiBk Yi1 pZDOid2R2ZDJ4MncOMmQ5eDNleHhhbng1ZZHB1cmR1ZHBOdGEyeGZ4Ij4yMDw va2V5PjwvZm9y ZWlnbi1rZXlzPjxyZWYtdHlwZSBuYWliPSJKb3VybmFsIEFydGljbGUiPjE3PC9yZW YtdHlwZT48 Y29udHJpYnVOb3JzPjxhdXRob3JzPjxhdXRob3I+VmlsbGFycm9lbCwgT S4gQy48L2F1 dGhvcj48 YXV0aG9yPlJhamVzaGt1bWFyLCBOLiBWLjwvYXV0aG9yPjxhdXRob3I+R2Fycmlk by1MYWd1bmEs IEkuPC9hdXRob3I+PGF1dGhvcj5EZSBKZXN1cy1BY29zdGEsIEEuPC9hdXRob3I+P GF1dGhvcj5K b251cywgUy48L2F1dGhvcj48YXV0aG9yPk1haXRyYSwgQS48L2F1dGhvcj48YXVOa G9yPkhydWJh biwgUi4gSC48L2F1dGhvcj48YXV0aG9yPkVzaGxlbWFuLCBKLiBSLjwvYXV0aG9y PjxhdXRob3I+S2xlaW4sIEEuPC9hdXRob3I+PGF1dGhvcj5MYWhlcnUsIEQuPC9hdXRob3I+PGF1d Ghvcj5Eb251 aG93ZXIsIFIuPC9hdXRob3I+PGF1dGhvcj5IaWRhbGdvLCBNLjwvYXV0aG9yPjwvY XV0aG9ycz48 L2NvbnRyaWJ1dG9ycz48YXVOaC1hZGRyZXNzPkNvcnJlc3BvbmRpbmcgQXV0aG9 yOiBNYW51ZWwg SGlkYWxnbywgQ2xpbmljYWwgUmVzZWFyY2ggUHJvZ3JhbSwgU3BhbmlzaCBOY XRpb25hbCBDYW5j ZXIgUmVzZWFyY2ggQ2VudGVyIChDTkIPKSwgTWVsY2hvciBGZXJuYW5kZXog QWxtYWdybywgMywg MjgwMjksIE 1hZHJpZCwgU3BhaW4uPC9hdXRoLWFkZHJlc3M+PHRpdGxlcz48dGl0 bGU+UGVyc29u YWxpemluZyBjYW5jZXIgdHJ1YXRtZW50IGluIHRoZSBhZ2Ugb2YgZ2xvYmFsIGdlb m9taWMgYW5h bHlzZXM6IFBBTEIyIGdlbmUgbXV0YXRpb25zIGFuZCBOaGUgcmVzcG9uc2UgdG8 gRE5BIGRhbWFn aW5nIGFnZW50cyBpbiBwYW5jcmVhdGljIGNhbmNlcjwvdGl0bGU+PHN1Y29uZGFy eS1OaXRsZT5N b2wgQ2FuY2VyIFRoZXI8L3N1Y29uZGFyeSOaXRsZT48YWx0LXRpdGxlPk1vbGVj dWxhciBjYW5j ZXIgdGhlcmFwZXV0aWNzPC9hbHQtdGl0bGU+PC90aXRsZXM+PHBlcmlvZGljYW w+PGZ1bGwtdGl0 bGU+TW9sIENhbmNlciBUaGVyPC9mdWxsLXRpdGxlPjxhYmJyLTE+TW9sZWN1b GFyIGNhbmNlciB0 aGVyYXB1dXRpY3M8L2FiYnItMT48L3B1cmlvZGljYWw+PGFsdC1wZXJpb2RpY2F sPjxmdWxsLXRp dGxlPk1vbCBDYW5jZXIgVGhlcjwvZnVsbC1OaXRsZT48YWJici0xPk1vbGVjdWxhci BjYW5jZXIg dGhlcmFwZXV0aWNzPC9hYmJyLTE+PC9hbHQtcGVyaW9kaWNhbD48cGFnZXM+My04PC9wYWdlcz48 dm9sdWllPjEwPC92b2x1bWU+PG51bWJlcj4xPC9udWliZXI+PGtleXdvcmRzPjxrZXl 3b3JkPkFu aW1hbHM8L2tleXdvcmQ+PGtleXdvcmQ+KkROQSBCcmVha3MsIERvdWJsZS1TdHJ hbmRlZDwva2V5 d29yZD48a2V5d29yZD5EZW94eWN5dGlkaW51L2FuYWxvZ3MgJmFtcDsgZGVyaXZ hdGl2ZXMvdGhl cmFwZXVOaWMgdXN1PC9rZX13b3JkPjxrZX13b3JkPkRpc2Vhc2UgUHJvZ3Jlc3Npb2 48L2tleXdv cmQ+PGtleXdvcmQ+RHJ1ZyBSZXNpc3RhbmN1LCBOZW9wbGFzbTwva2V5d29yZ D48a2V5d29yZD5G ZW1hbGU8L2tleXdvcmQ+PGtleXdvcmQ+SHVtYW5zPC9rZX13b3JkPjxrZX13b3JkPkl uZG12aWR1 YWxpemVkIE11ZGljaW51L21ldGhvZHM8L2tleXdvcmQ+PGtleXdvcmQ+TWFsZTwva 2V5d29yZD48 a2V5d29yZD5NaWN1PC9rZX13b3JkPjxrZX13b3JkPk1pY2UsIE51ZGU8L2tleXdvcmQ+PGtleXdv cmQ+TW1kZGxlIEFnZWQ8L2tleXdvcmQ+PGtleXdvcmQ+TW1Ob215Y2luLyp0aGVy YXB1dXRpYyB1 c2U8L2tleXdvcmQ+PGtleXdvcmQ+TXV0YXRpb248L2tleXdvcmQ+PGtleXdvcmQ+T nVjbGVhciBQ cm90ZWlucy8qZ2VuZXRpY3M8L2tleXdvcmQ+PGtleXdvcmQ+UGFuY3JIYXRpYyB OZW9wbGFzbXMv KmRydWcgdGhlcmFweS8qZ2VuZXRpY3MvcGF0aG9sb2d5PC9rZX13b3JkPjxrZXl3b 3JkP1R1bW9y IFN1cHByZXNzb3IgUHJvdGVpbnMvKmdlbmV0aWNzPC9rZX13b3JkPjxrZX13b3JkPI hlbm9ncmFm dCBNb2R1bCBBbnRpdHVtb3IgQXNzYX1zPC9rZX13b3JkPjwva2V5d29yZHM+PGRh dGVzPjx5ZWFy PjIwMTE8L311YXI+PHB1Yi1kYXRlcz48ZGF0ZT5KYW48L2RhdGU+PC9wdWItZGF OZXM+PC9kYXR1 cz48aXNibj4xNTM4LTg1MTQgKEVsZWN0cm9uaWMpJiN4RDsxNTM1L TcxNjMgK ExpbmtpbmcpPC9p c2JuPjxhY2Nlc3Npb24tbnVtPjIxMTM1MjUxPC9hY2Nlc3Npb24tbnVtPjxl cmxzPjxyZ WxhdGVk LXVybHM+PHVybD5odHRwOi8vd3d3Lm5jYmkubmxtLm5paC5nb3YvcHVibWVkLz IxMTM1MjUxPC91 cmw+PC9yZWxhdGVkLXVybHM+PC91cmxzPjxj dXN0b20yPjMzMDczNDA8L2N1c 3RvbTI+PGVsZWN0 cm9uaWMtcmVzb3VyY2UtbnVtPjEwLjExNTgvMTUzNSO3MTYzLk1DVC0xMC0wO DkzPC91bGVjdHJv bmljLXJlc291cmN1LW51bT48L3J1Y29yZD48LONpdGU+PC9FbmROb3R1Pn==PEVuZE5vdGU+PENpdGU+PEF1dGhvcj5Gb2dlbG1hbjwvQXV0aG9yPjxZZWFyPjIw MTE8L111YXI+PFJIY051bT4yMTwvUmVj TnVtPjxEaXNwbGF5VGV4dD48c3R5bGUgZmFjZT0ic3V wZXJzY3JpcHQi PjE1LTE4PC9zdH1sZT48L0Rpc3BsYX1UZXhOPjxyZWNvcmQ+PHJIYyludW liZXI+MjE8L3JIYylu dW1iZXI+PGZvcmVpZ24ta2V5cz48a2V5IGFwcDOiRU4iIGRiLW1kPSJ3ZHZkMngydz QyZD14M2V4 eGFueDVkcGVyZGVkcHROYTJ4ZngiPjIxPC9rZXk+PC9mb3J1aWduLWtleXM+PHJIZ i10eXBlIG5h bWU9IkpvdXJuYWwgQXJOaWNsZSI+MTc8L3JZiOeXBIPjxjb250cmlidXRvcnM+P GF1 dGhvcnM+PGF1dGhvcj5Gb2dlbG1hbiwgRC4gUi48L2F1dGhvcj48YXV0aG9yPldvbGZmLCBSLi BBLjwvYXVO aG9yPjxhdXRob3I+S29wZXR6LCBTLjwvYXV0aG9yPjxhdXRob3I+SmF2bGUsIEOuP C9hdXRob3I+PGF1dGhvcj5CcmFkbGV5LCBDLjwvYXV0aG9yPjxhdXRob3I+TW9rLCBJLjwvYXV 0aG9yPjxhdXRo b3I+Q2FiYW5pbGxhcywgRi48L2F1dGhvcj48YXV0aG9yPkFiYnJ1enplc2UsIEouIEwu PC9hdXRo b3I+PC9hdXRob3JzPjwvY29udHJpYnVOb3JzPjxhdXRoLWFkZHJlc3M+T S5ELiBBb mRlcnNvbiBD YW5jZXIgQ2VudGVyLCBIb3VzdG9uLCBUWCA3NzAzMCwgVVNBLiBkZm9nZW xtYWSAbWRhbmRlcnNv bi5vcmc8L2F1dGgtYWRkcmVzcz48dGl0bGVzPjx0aXRsZT5FdmlkZW5jZSBmb3IgdG hlIGVmZmlj YWN5IG9mIEluaXBhcmliLCBhIFBBUlAtMSBpbmhpYm10b3IsIGluIEJSQOEyLWFzc 29jaWF0ZWQg cGFuY3JIYXRpYyBjYW5jZXI8L3RpdGxlPjxzZWNvbmRhcnktdGl0bGU+QW50aWN hbmNlciBSZXM8 L3N1Y29uZGFyeS10aXRsZT48YWx0LXRpdGxlPkFudGljYW5jZXIgcmVzZWFyY2g 8L2FsdC10aXRs ZT48L3RpdGxlcz48cGVyaW9kaWNhbD48ZnVsbC10aXRsZT5BbnRpY2FuY2VyIFJIc zwvZnVsbC10 aXRsZT48YWJici0xPkFudGljYW5jZXIgcmVzZWFyY2g8L2FiYnItMT48L3B1cmlvZG ljYWw+PGFs dC1wZXJpb2RpY2FsPjxmdWxsLXRpdGxlPkFudGljYW5jZXIgUmVzPC9mdWxsLXR pdGxlPjxhYmJy LTE+QW50aWNhbmNIciByZXN1YXJjaDwvYWJici0xPjwvYWx0LXBlcmlvZGljYW w+PHBhZ2VzPjE0 MTctMjA8L3BhZ2VzPjx2b2x1bWU+MzE8L3ZvbHVtZT48bnVtYmVyPjQ8L251bWJl cj48a2V5d29y ZHM+PGtleXdvcmQ+QWRlbm9jYXJjaW5vbWEvZ2VuZXRpY3MvKnN1Y29uZGFye S90aGVyYXB5PC9r ZX13b3JkPjxrZX13b3JkPkJSQOEyIFByb3RlaW4vKmdlbmV0aWNzPC9rZX13b3JkPjxr ZX13b3Jk PkJlbnphbWlkZXMvKnRoZXJhcGV1 dGljIHVzZTwva2V5d29yZD48a2V5d29yZD5Cc mVhc3QgTmVv cGxhc21zL2dlbmV0aWNzLypwYXRob2xvZ3kvdGhlcmFweTwva2V5d29yZD48a2V5d 29yZD5GZW1h bGU8L2tleXdvcmQ+PGtleXdvcmQ+R2VybS1MaW51IE11dGF0aW9uL2dlbmV0aWNz PC9rZX13b3Jk PjxrZX13b3JkPkhlbWFuczwva2V5d29yZD48a2V5d29yZD5NaWRkbGUgQWdIZDwva 2V5d29yZD48 a2V5d29yZD50ZW9wbGFzb SBSZWN1cnJlbmNILCBMb2NhbC9nZW5ldGljcy8qcGF 0aG9sb2d5L3Ro ZXJhcHk8L2tleXdvcmQ+PGtleXdvcmQ+UGFuY3JIYXRpYyBOZW9wbGFzbXMvZ2 VuZXRpY3MvKnN1 Y29uZGFyeS90aGVyYXB5PC9rZX13b3JkPjxrZX13b3JkP1BvbHkoQURQLXJpYm9zZ SkgUG9seW11 cmFzZXMvKmFudGFnb25pc3RzICZhbXA7IGluaGliaXRvcnM8L2tleXdvcmQ+PGtleX dvcmQ+VHJI YXRtZW50IE91dGNvbWU8L2tleXdvcmQ+PC9rZX13b3Jkcz48ZGF0ZXM+PHIIYXI+MjAxMTwveWVh cj48cHViLWRhdGVzPjxkYXRlPkFwcjwvZGF0ZT48L3B1Yi1kYXR1cz48L2RhdGVzP jxpc2JuPjE3 OTEtNzUzMCAoRWx1Y3Ryb25pYykmI3hEOzAyNTAtNzAwNSAoTGlua2luZyk8L21 zYm4+PGFjY2Vz c2lvbiludWO+MjE1MDgzOTU8L2FjY2Vzc2lvbiudWO+PHVybHM+PHJ1bGFOZWQt dXJscz48dXJs PmhOdHA6Ly93d3cubmNiaS5ubGOubmloLmdvdi9wdWJtZWQvMjE1MDgzOTU8L3V ybD48L3J1bGF0 ZWQtdXJscz48L3VybHM+PC9yZWNvcmQ+PC9DaXR1PjxDaXRIPjxBdXRob3I+Rm9 uZzwvQXV0aG9y PjxZZWFyPjIwMDk8L 111YXI+PFJ1Y051bT4xMDwvUmVj TnVtPjxyZWNvcmQ+PHJI YyludW liZXI+MTA8L3JIYyludW liZXI+PGZvcmVpZ24ta2V5cz48a2V5IGFwcDOiRU4iIGRiLW1kP SJ3ZHZkMngy dzQyZD14M2V4eGFueDVkcGVyZGVkcHROYTJ4ZngiPjEwPC9rZXk+PC9mb3JlaWd uLWtleXM+PHJI Zi1OeXBlIG5hbWU9IkpvdXJuYWwgQXJOaWNsZSI+MTc8L3JZiOeXBIPjxjb250cml idXRvcnM+PGF1dGhvcnM+PGF1dGhvcj5Gb25nLCBQLiBDLjwvYXV0aG9yPjxhdXRob3I+Qm9z cywgRC4gUy48 L2F1dGhvcj48YXV0aG9yPllhcCwgVC4gQS48L2F1dGhvcj48YXV0aG9yP1R1dHQsIE EuPC9hdXRo b3I+PGF1dGhvcj5XdSwgUC48L2F1dGhvcj48YXV0aG9yPk1lcmdl aS1Sb2Vsdmluayw gTS48L2F1 dGhvcj48YXV0aG9yPk1vcnRpbWVyLCBQLjwvYXV0aG9yPjxhdXRob3I+U3dhaXNs YW5kLCBILjwv YXV0aG9yPjxhdXRob3I+TGF1LCBBLjwvYXV0aG9yPjxhdXRob3I+TyZhcG9zOONv bm5vciwgTS4g Si48L2F1dGhvcj48YXV0aG9yPkFzaHdvcnRoLCBBLjwvYXV0aG9yPjxhdXRob3I+Q 2FybWljaGF1 bCwgSi48L2F1dGhvcj48YXV0aG9yPktheWUsIFMuIEIuPC9hdXRob3I+PGF1dGhvcj5 TY2hlbGxl bnMsIEouIEguPC9hdXRob3I+PGF1dGhvcj5kZSBCb25vLCBKLiBTLjwvYXV0aG9yP jwvYXV0aG9y cz48L2NvbnRyaWJ1dG9ycz48YXVOaC1hZGRyZXNzPkRydWcgRGV2ZWxvcGllbnQ gVW5pdCwgUm95 YWwgTWFyc2RlbiBOYXRpb25hbCBIZWFsdGggU2VydmljZ SBGb3VuZGFOaW9uIF RydXN0IGFuZCB0 aGUgSW5zdG1OdXR1IG9mIENhbmNlciBSZXNYXJjaCwgU3VOdG9uLCBTdXJyZXk sIFVuaXR1ZCBL aW5nZG9tLjwvYXVOaC1hZGRyZXNzPjx0aXRsZXM+PHRpdGxlPkluaGliaXRpb24g b2YgcG9seShB RFAtcmlib3N1KSBwb2x5bWVyYXN1IGluIHR1bW9ycyBmcm9tIEJSQOEgbXV0YXRp b24gY2Fycmll cnM8L3RpdGxlPjxzZWNvbmRhcnktdGl0bGU+TiBFbmdsIEogTWVkPC9zZWNvbmR hcnktdGl0bGU+PGFsdC1OaXRsZT5UaGUgTmV3IEVuZ2xhbmQgam91cm5hbCBvZiBtZWRpY2luZT wvYWx0LXRpdGxl PjwvdGl0bGVzPjxwZXJpb2RpY2FsPjxmdWxsLXRpdGxlPk4gRW5nbCBKIE llZDwv ZnVsbC1OaXRs ZT48YWJici0xPlRoZSBOZXcgRW5nbGFuZCBqb3VybmFsIG9mIG11ZGljaW51PC9h YmJyLTE+PC9w ZXJpb2RpY2FsPjxhbHQtcGVyaW9kaWNhbD48ZnVsbC1OaXRsZT50IEVuZ2wgSiBN ZWQ8L2Z1bGwt dGl0bGU+PGFiYnItMT5UaGUgTmV3IEVuZ2xhbmQgam91cm5hbCBvZiBtZWRpY21 uZTwvYWJiciOx PjwvYWx0LXBlcmlvZGljYWw+PHBhZ2VzPjEyMyOzNDwvcGFnZXM+PHZvbHVtZ T4zNjE8L3ZvbHVt ZT48bnVtYmVyPjI8L251bWJlcj48a2V5d29yZHM+PGtleXdvcmQ+QWR1bHQ8L2tle XdvcmQ+PGtl eXdvcmQ+QWd1ZDwva2V5d29yZD48a2V5d29yZD5BZ2VkLCA4MCBhbmQgb3Zlcj wva2V5d29yZD48 a2V5d29yZD5Eb3N1LVJlc3BvbnNlIFJlbGFOaW9uc2hpcCwgRHJ1Zzwva2V5d29yZD4 8a2V5d29y ZD5EcnVnIERlc2lnbjwva2V5d29yZD48a2V5d29yZD5GZW1hbGU8L2tleXdvcmQ+P GtleXdvcmQ+KkdlbmVzLCBCUkNBMTwva2V5d29yZD48a2V5d29yZD4qR2VuZXMsIEJSQOEyPC 9rZX13b3JkPjxr ZX13b3JkPkdlcmOtTGluZSBNdXRhdGlvbjwva2V5d29yZD48a2V5d29yZD5IZXRlcm9 6eWdvdGU8 L2tleXdvcmQ+PGtleXdvcmQ+SHVtYW5zPC9rZX13b3JkPjxrZX13b3JkPk1hbGU8L2tl eXdvcmQ+PGtleXdvcmQ+TW1kZGx1IEFnZWQ8L2tleXdvcmQ+PGtleXdvcmQ+TmVvcGxhc21zL ypkcnVnIHiRo ZXJhcHkvZ2VuZXRpY3M8L2tleXdvcmQ+PGtleXdvcmQ+UGh0aGFsYXppbmVzL2F kdmVyc2UgZWZm ZWNOcy9waGFybWFjb2tpbmV0aWNzLypOaGVyYXBldXRpYyB1c2U8L2tleXdvcmQ+PGtleXdvcmQ+UG1wZXJhemluZXMvYWR2ZXJzZ SB1ZmZ1Y3RzL3BoYXJtYWNva2luZXRpY3MvK nRoZXJhcGV 1dGlj IHVzZTwva2V5d29yZD48a2V5d29yZD5Qb2x5KEFEUC1yaWJvc2UpIFBvbHltZXJhc 2VzLyphbnRh Z29uaXNOcyAmYW1wOyBpbmhpYm10b3JzPC9rZX13b3JkPjxrZX13b3JkP11vdW5nIEF kdWxOPC9r ZX13b3JkPjwva2V5d29yZHM+PGRhdGVzPjx5ZWFyPjIwMDk8L311YXI+PHB1Yi1kY XR1cz48ZGF0 ZT5KdWwgOTwvZGF0ZT48L3B1Yi1kYXR1cz48L2RhdGVzPjxpc2JuPjElMzMtNDQ wNiAoRWxIY3Ry b25pYykmI3hEOzAwMjgtNDc5MyAoTGlua2luZyk8L2lzYm4+PGFjY2Vzc2lvbiludW O+MTk1NTM2 NDE8L2FjY2Vzc2lvbiludWO+PHVybHM+PHJIbGFOZWQtdXJscz48dXJsPmhOdHA6 Ly93d3cubmNi aS5ubGOubmloLmdvdi9wdWJtZWQvMTk1NTM2NDE8L3VybD48L3JIbGFOZWQtdX Jscz48L3VybHM+PGVsZWN0cm9uaWMtcmVzb3VyY2UtbnVtPjEwLjEwNTYvTkVKTW9hMDkwMDI xMjwvZWxlY3Ryb25p Yy 1yZXNvdXJjZS1udWO+PC9yZWNvcmQ+PC9DaXRIPjxDaXRIPjxBdXRob3I+dmF uIGRIciBIZWlq ZGVuPC9BdXRob3I+PFIIYXI+MjAwNTwvWWVhcj48UmVjTnVtPjE3PC9SZWNOd WO+PHJIY29yZD48 cmVjLW51bWJlcj4xNzwvcmVjLW51bWJlcj48Zm9yZWlnbi1rZXIzPjxrZXkgYXBwPS JFTiIgZGIt aWQ9IndkdmQyeDJ3NDJkOXgzZXh4YW54NWRwZXJkZWRwdHRhMnhmeCI+MTc 8L2tleT48L2ZvcmVp Z24ta2V5cz48cmVmLXR5cGUgbmFtZT0iSm91cm5hbCBBcnRpY2xlIj4xNzwvcmVm LXR5cGU+PGNv bnRyaWJ1dG9ycz48YXV0aG9ycz48YXV0aG9yPnZhbiBkZXIgSGVpamRlbiwgT S4gU y48L2F1dGhv cj48YXV0aG9yPkJyb2R5LCBKLiBSLjwvYXV0aG9yPjxhdXRob3I+RGV6ZW50amU sIEQuIEEuPC9h dXRob3I+PGF1dGhvcj5HYWxsbWVpZXIsIEUuPC9hdXRob3I+PGF1dGhvcj5DdW5u aW5naGFtLCBT LiBDLjwvYXV0aG9yPjxhdXRob3I+U3dhcnR6LCBNLiBKLjwvYXV0aG9yPjxhdXRo b3I+RGVNYXJ6 bywgQS4gTS48L2F1dGhvcj48YXV0aG9yPk9mZmVyaGF 1 cywgRy4g Si48L2F1dGhvc j48YXV0aG9y PklzYWNvZmYsIFcuIEguPC9hdXRob3I+PGF1dGhvcj5IcnViYW4sIFIuIEguPC9hdXR ob3I+PGF1 dGhvcj5LZXJuLCBTLiBFLjwvYXV0aG9yPjwvYXV0aG9ycz48L2NvbnRyaWJ1dG9y cz48YXVOaClh ZGRyZXNzPkRlcGFydG1lbnQgb2YgT25jb2xvZ3ksIFRoZSBTb2wgR29sZG1hbiBQY W5jcmVhdGlj IENhbmNlciBSZXN1YXJjaCBDZW50ZXIsIFRoZSBKb2hucyBIb3BraW5zIFVuaXZlcn NpdHkgU2No b29sIG9mIE11ZG1jaW51LCBCYWxOaW1vemUslE1henlsYW5kIDIxMjMxLCBVUOEu PC9hdXRoLWFk ZHJlc3M+PHRpdGxlcz48dGl0bGU+SW4gdm12byB0aGVyYXBldXRpYyByZXNwb25 zZXMgY29udGlu Z2VudCBvbiBGYW5jb25pIGFuZW1pYS9CUkNBMiBzdGFOdXMgb2YgdGhlIHR1b W9yPC9OaXRsZT48 c2Vjb25kYXJ5LXRpdGxlPkNsaW4gQ2FuY2VyIFJlczwvc2Vjb25kYXJ5LXRpdGxlPjx hbHQtdGlO bGU+Q2xpbmljYWwgY2FuY2VyIHJlc2VhcmNoIDogYW4gb2ZmaWNpYWwgam91c m5hbCBvZiBOaGUg QWllcmljYW4gQXNzb2NpYXRpb24gZm9yIENhbmNlciBSZXN1YXJjaDwvYWxOLX RpdGxlPjwvdGlO bGVzPjxwZXJpb2RpY2FsPjxmdWxsLXRpdGxlPkNsaW4gQ2FuY2VyIFJlczwvZnVsb ClOaXRsZT48 YWJiciOxPkNsaW5pY2FsIGNhbmNlciByZXN1YXJjaCA6IGFuIG9mZmljaWFsIGpvdX JuYWwgb2Yg dGhlIEFtZXJpY2FuIEFzc29jaWFOaW9uIGZvciBDYW5jZXIgUmVzZWFyY2g8L2FiY nItMT48L3B1 cmlvZGljYWw+PGFsdC1wZXJpb2RpY2FsPjxmdWxsLXRpdGxlPkNsaW4gQ2FuY2V yIFJlczwvZnVs bC1OaXRsZT48YWJiciOxPkNsaW5pY2FsIGNhbmNlciByZXN1YXJjaCA6IGFuIG9mZ mljaWFsIGpv dXJuYWwgb2YgdGhlIEFtZXJpY2FuIEFzc29jaWFOaW9uIGZvciBDYW5jZXIgUmVz ZWFyY2g8L2Fi YnItMT48L2FsdC1wZXJpb2RpY2FsPjxwYWdlcz43NTA4LTE1PC9wYWdlcz48dm9s dWllPjExPC92 b2x1bWU+PG51bWJlcj4yMDwvbnVtYmVyPjxrZX13b3Jkcz48a2V5d29yZD5BbmltY WxzPC9rZX13 b3JkPjxrZX13b3JkPkFudGluZW9wbGFzdGljIEFnZW50cy9waGFybWFjb2xvZ3k8L2tle XdvcmQ+PGtleXdvcmQ+QXBvcHRvc2lzL2RydWcgZWZmZWNOczwva2V5d29yZD48a2V5d29 yZD5CUkNBMiBQ cm90ZWluL2RlZmljaWVuY3kvZ2VuZXRpY3M8L2tleXdvcmQ+PGtleXdvcmQ+Q2Fz cGFzZXMvbWVO YWJvbGlzbTwva2V5d29yZD48a2V5d29yZD5DZWxsIEN5Y2x1L2RydWcgZWZmZW NOczwva2V5d29y ZD48a2V5d29yZD5DZWxsIExpbmUsIFR1bW9yPC9rZX13b3JkPjxrZX13b3JkPkN1bG wgU3Vydml2 YWwvZHJ1ZyB1ZmZ1Y3RzPC9rZX13b3JkPjxrZX13b3JkPkNobG9yYWlidWNpbC9wa GFybWFjb2xv Z3k8L2tleXdvcmQ+PGtleXdvcmQ+Q2lzcGxhdGluL3BoYXJtYWNvbG9neTwva2V5d 29yZD48a2V5 d29yZD5Dcm9zcy1MaW5raW5nIFJlYWdlbnRzLypwaGFybWFjb2xvZ3kvdGhlcmFwZ XVOaWMgdXN1 PC9rZX13b3JkPjxrZX13b3JkPkRlb3h5Y310aWRpbmUvYW5hbG9ncyAmYW1wOyBkZ XJpdmFOaXZ1 cy9waGFybWFjb2xvZ3k8L2tleXdvcmQ+PGtleXdvcmQ+RG9zZSISZXNwb25zZSBSZ WxhdGlvbnNo aXAsIERydWc8L2tleXdvcmQ+PGtleXdvcmQ+RG94b3J1YmljaW4vcGhhcm1hY29sb2 d5PC9rZX13 b3JkPjxrZX13b3JkPkVOb3Bvc2lkZS9waGFybWFjb2xvZ3k8L2tleXdvcmQ+PGtleXdvc mQ+RmFu Y29uaSBBbmVtaWEgQ29tcGxlbWVudGF0aW9uIEdyb3VwIEMgUHJvdGVpbi9kZW ZpY211bmN5L2dl bmV0aWNzPC9rZX13b3JkPjxrZX13b3JkPkZhbmNvbmkgQW5lbW1hIENvbXBsZWlb nRhdGlvbiBH cm91cCBHIFByb3R1aW4vZGVmaWNpZW5jeS9nZW5ldGljczwva2V5d29yZD48a2V5 d29yZD5GYW5j b25pIEFuZW1pYSBDb21wbGVtZW50YXRpb24gR3JvdXAgUHJvdGVpbnMvZGVma WNpZW5jeS8qZ2Vu ZXRpY3M8L2tleXdvcmQ+PGtleXdvcmQ+RmVtYWx1PC9rZX13b3JkPjxrZX13b3JkPk ZsdW9yb3Vy YWNpbC9waGFybWFjb2xvZ3k8L2tleXdvcmQ+PGtleXdvcmQ+SHVtYW5zPC9rZX13 b3JkPjxrZX13 b3JkPkluaGliaXRvcnkgQ29uY2VudHJhdGlvbiA1MDwva2V5d29yZD48a2V5d29yZD5 NZWxwaGFs YW4vcGhhcm1hY29sb2d5PC9rZX13b3JkPjxrZX13b3JkPk1pY2U8L2tleXdvcmQ+PGtl eXdvcmQ+TWIjZSwgTnVkZTwva2V5d29yZD48a2V5d29yZD5NaXRvbXljaW4vKnBoYXJtYWN vbG9neS90aGVy YXBIdXRpYyB1c2U8L2tleXdvcmQ+PGtleXdvcmQ+TXV0YXRpb248L2tleXdvcmQ+PGtleXdvcmQ+UGFjbG1OYXh1bC9waGFybWFjb2xvZ3k8L2tleXdvcmQ+PGtleXdvcmQ+UGFuY3JlY XRpYyBOZW9w bGFzbXMvKmRydWcgdGhlcmFweS9nZW5ldGljcy9wYXRob2xvZ3k8L2tleXdvcmQ+PGtleXdvcmQ+VG1tZSBGYWNOb3JzPC9rZX13b3JkPjxrZX13b3JkPlZpbmJsYXNOaW51L3BoYXJtYW NvbG9neTwv a2V5d29yZD48a2V5d29yZD5YZW5vZ3JhZnQgTW9kZWwgQW50aXR1bW9yIEFzc2 F5cy9tZXRob2Rz PC9rZX13b3JkPjwva2V5d29yZHM+PGRhdGVzPjx5ZWFyPjIwMDU8L311YXI+PHB1 YilkYXRlcz48 ZGF0ZT5PY3QgMTU8L2RhdGU+PC9wdWItZGF0ZXM+PC9kYXRlcz48aXNibj4xM Dc4LTA0MzIgKFBy aW50KSYjeEQ7MTA3OC0wNDMyIChMaW5raW5nKTwvaXNibj48YWNjZXNzaW9 uLW51bT4xNjI0Mzgy NTwvYWNjZXNzaW9uLW51bT48dXJscz48cmVsYXR1ZC11cmxzPjxl cmw+aHR0cD ovL3d3dy5uY2Jp Lm5sbS5uaWguZ292L3B1Ym11ZC8xNjIOMzgyNTwvdXJsPjwvcmVsYXR1ZC11cmxz PjwvdXJscz48 ZWxlY3Ryb25pYy1yZXNvdXJjZSiudWO+MTAuMTE1OC8xMDc4LTAOMzIuQONS LTA1LTEwNDg8L2Vs ZWN0cm9uaWMtcmVzb3VyY2UtbnVtPjwvcmVjb3JkPjwvQ210ZT48Q210ZT48QXV0 aG9yPlZpbGxh cnJvZWw8LOF1dGhvcj48WWVhcj4yMDExPC9ZZWFyPjxSZWNOdW0+MjA8L1J1Y 05lbT48cmVjb3Jk PjxyZWMtbnVtYmVyPjIwPC9yZWMtbnVtYmVyPjxmb3JlaWduLWtleXM+PGtleSBh cHA9IkV0IiBk Yi1 pZD0id2R2ZDJ4MncOMmQ5eDNleHhhbng1ZZHB1cmR1ZHBOdGEyeGZ4Ij4yMDw va2V5PjwvZm9y ZWlnbi1rZXlzPjxyZWYtdHlwZSBuYWliPSJKb3VybmFsIEFydGljbGUiPjE3PC9yZW YtdHlwZT48 Y29udHJpYnVOb3JzPjxhdXRob3JzPjxhdXRob3I+VmlsbGFycm9lbCwgT S4gQy48L2F1 dGhvcj48 YXV0aG9yPlJhamVzaGt1bWFyLCBOLiBWLjwvYXV0aG9yPjxhdXRob3I+R2Fycmlk by1MYWd1bmEs IEkuPC9hdXRob3I+PGF1dGhvcj5EZSBKZXN1cy1BY29zdGEsIEEuPC9hdXRob3I+P GF1dGhvcj5K b251cywgUy48L2F1dGhvcj48YXV0aG9yPk1haXRyYSwgQS48L2F1dGhvcj48YXVOa G9yPkhydWJh biwgUi4gSC48L2F1dGhvcj48YXV0aG9yPkVzaGxlbWFuLCBKLiBSLjwvYXV0aG9y PjxhdXRob3I+S2xlaW4sIEEuPC9hdXRob3I+PGF1dGhvcj5MYWhlcnUsIEQuPC9hdXRob3I+PGF1d Ghvcj5Eb251 aG93ZXIsIFIuPC9hdXRob3I+PGF1dGhvcj5IaWRhbGdvLCBNLjwvYXV0aG9yPjwvY XV0aG9ycz48 L2NvbnRyaWJ1dG9ycz48YXV0aC1hZGRyZXNzPkNvcnJlc3BvbmRpbmcgQXV0aG9 yOiBNYW51ZWwg SGlkYWxnbywgQ2xpbmljYWwgUmVzZWFyY2ggUHJvZ3JhbSwgU3BhbmlzaCBOY XRpb25hbCBDYW5j ZXIgUmVzZWFyY2ggQ2VudGVyIChDTk1PKSwgTWVsY2hvciBGZXJuYW5kZXog QWxtYWdybywgMywg MjgwMjksIE 1hZHJpZCwgU3BhaW4uPC9hdXRoLWFkZHJ1c3M+PHRpdGxlcz48dGl0 bGU+UGVyc29u YWxpemluZyBjYW5jZXIgdHJ1YXRtZW50IGluIHRoZSBhZ2Ugb2YgZ2xvYmFsIGdlb m9taWMgYW5h bH1zZXM6IFBBTEIyIGdlbmUgbXV0YXRpb25zIGFuZCBOaGUgcmVzcG9uc2UgdG8 gRE5BIGRhbWFn aW5nIGFnZW50cyBpbiBwYW5jcmVhdGljIGNhbmNlcjwvdGl0bGU+PHN1Y29uZGFy eS10aXRsZT5N b2wgQ2FuY2VyIFRoZXI8L3N1Y29uZGFyeSOaXRsZT48YWx0LXRpdGxlPk1vbGVj dWxhciBjYW5j ZXIgdGhlcmFwZXV0aWNzPC9hbHQtdGl0bGU+PC90aXRsZXM+PHBlcmlvZGljYW w+PGZ1bGwtdGl0 bGU+TW9sIENhbmNlciBUaGVyPC9mdWxsLXRpdGxlPjxhYmJyLTE+TW9sZWN1b GFyIGNhbmNlciB0 aGVyYXB1dXRpY3M8L2FiYnItMT48L3B1cmlvZGljYWw+PGFsdC1wZXJpb2RpY2F sPjxmdWxsLXRp dGxlPk1vbCBDYW5jZXIgVGhlcjwvZnVsbC1OaXRsZT48YWJici0xPk1vbGVjdWxhci BjYW5jZXIg dGhlcmFwZXV0aWNzPC9hYmJyLTE+PC9hbHQtcGVyaW9kaWNhbD48cGFnZXM+My04PC9wYWdlcz48 dm9sdWllPjEwPC92b2x1bWU+PG51bWJlcj4xPC9udWliZXI+PGtleXdvcmRzPjxrZXl 3b3JkPkFu aW1hbHM8L2tleXdvcmQ+PGtleXdvcmQ+KkROQSBCcmVha3MsIERvdWJsZS1TdHJ hbmRlZDwva2V5 d29yZD48a2V5d29yZD5EZW94eWN5dG1kaW51L2FuYWxvZ3MgJmFtcDsgZGVyaXZ hdG12ZXMvdGhl cmFwZXV0aWMgdXN1PC9rZX13b3JkPjxrZX13b3JkPkRpc2Vhc2UgUHJvZ3Jlc3Npb2 48L2tleXdv cmQ+PGtleXdvcmQ+RHJ1ZyBSZXNpc3RhbmN1LCBOZW9wbGFzbTwva2V5d29yZ D48a2V5d29yZD5G ZW1hbGU8L2tleXdvcmQ+PGtleXdvcmQ+SHVtYW5zPC9rZX13b3JkPjxrZX13b3JkPkl uZG12aWR1 YWxpemVkIE1lZGljaW51L21ldGhvZHM8L2tleXdvcmQ+PGtleXdvcmQ+TWFsZTwva 2V5d29yZD48 a2V5d29yZD5NaWN1PC9rZX13b3JkPjxrZX13b3JkPk1pY2UsIE51ZGU8L2tleXdvcmQ+PGtleXdv cmQ+TW1kZGx1IEFnZWQ8L2tleXdvcmQ+PGtleXdvcmQ+TW1Ob215Y2luLyp0aGVy YXB1dXRpYyB1 c2U8L2tleXdvcmQ+PGtleXdvcmQ+TXV0YXRpb248L2tleXdvcmQ+PGtleXdvcmQ+T nVjbGVhciBQ cm90ZWlucy8qZ2VuZXRpY3M8L2tleXdvcmQ+PGtleXdvcmQ+UGFuY3JIYXRpYyB OZW9wbGFzbXMv KmRydWcgdGhlcmFweS8qZ2VuZXRpY3MvcGF0aG9sb2d5PC9rZX13b3JkPjxrZX13b 3JkP1R1bW9y IFN1cHByZXNzb3IgUHJvdGVpbnMvKmdlbmV0aWNzPC9rZX13b3JkPjxrZX13b3JkPI hlbm9ncmFm dCBNb2R1bCBBbnRpdHVtb3IgQXNzYX1zPC9rZX13b3JkPjwva2V5d29yZHM+PGRh dGVzPjx5ZWFy PjIwMTE8L311YXI+PHB1Yi1kYXR1cz48ZGF0ZT5KYW48L2RhdGU+PC9wdWItZGF OZXM+PC9kYXR1 cz48aXNibj4xNTM4LTg1MTQgKEVsZWN0cm9uaWMpJiN4RDsxNTM1L TcxNjMgK ExpbmtpbmcpPC9p c2JuPjxhY2Nlc3Npb24tbnVtPjIxMTM1MjUxPC9hY2Nlc3Npb24tbnVtPjxl cmxzPjxyZ WxhdGVk LXVybHM+PHVybD5odHRwOi8vd3d3Lm5jYmkubmxtLm5paC5nb3YvcHVibWVkLz IxMTM1MjUxPC91 cmw+PC9yZWxhdGVkLXVybHM+PC91cmxzPjxj dXN0b20yPjMzMDczNDA8L2N1c 3RvbTI+PGVsZWN0 cm9uaWMtcmVzb3VyY2UtbnVtPjEwLjExNTgvMTUzNSO3MTYzLk1DVCOxMCOwO DkzPC9lbGVjdHJv bmljLXJlc291cmNlLW51bT48L3J1Y29yZD48LONpdGU+PC9FbmROb3R1Pn==15-18. Trastuzumab has demonstrated therapeutic efficacy against GI tumors with ERBB2 amplification19 and is currently being evaluated in clinical trials for patients with metastatic colorectal cancerPEVuZE5vdGU+PENpdGU+PEF1dGhvcj5SYW1hbmFOaGFuPC9BdXRob3I+PF llYXI+MjAwNDwvWWVh cj48UmVjTnVtPjE4PC9SZWNOdW0+PERpc3BsYX1UZXhOPjxzdHlsZSBmYWNlPSJ zdXBlcnNjcmlw dCI+MjA8L3N0eWxlPjwvRGlzcGxheVRleHQ+PHJlY29yZD48cmVjLW51bWJlcj4xO DwvcmVjLW51 bWJlcj48Zm9yZWlnbi1rZXlzPjxrZXkgYXBwPSJFTiIgZGItaWQ9IndkdmQyeDJ3NDJ kOXgzZXh4 YW54NWRwZXJkZWRwdHRhMnhmeCI+MTg8L2tleT48L2ZvcmVpZ24ta2V5cz48c mVmLXR5cGUgbmFt ZT0iSm91cm5hbCBBcnRpY2xlIj4xNzwvcmVmLXR5cGU+PGNvbnRyaWJ1dG9ycz48 YXV0aG9ycz48 YXV0aG9yPlJhbWFuYXRoYW4sIFIuIEsuPC9hdXRob3I+PGF1dGhvcj5Id2FuZywgSi 4gSi48L2F1 dGhvcj48YXV0aG9yPlphbWJvbmksIFcuIEMuPC9hdXRob3I+PGF1dGhvcj5TaW5pY3 JvcGUsIEYu IEEuPC9hdXRob3I+PGF1dGhvcj5TYWZyYW4sIEguPC9hdXRob3I+PGF1dGhvcj5Xb 25nLCBNLiBL LjwvYXV0aG9yPjxhdXRob3I+RWFybGUsIEOuPC9hdXRob3I+PGF1dGhvcj5CcnVmc 2t5LCBBLjwv YXV0aG9yPjxhdXRob3I+RXZhbnMsIFQuPC9hdXRob3I+PGF1dGhvcj5Ucm9ldHNja GVsLCBNLjwv YXV0aG9yPjxhdXRob3I+V2Fsa28sIEMuPC9hdXRob3I+PGF1dGhvcj5EYXksIFIuPC9 hdXRob3I+PGF1dGhvcj5DaGVuLCBILiBYLjwvYXV0aG9yPjxhdXRob3I+Rmlua2Vsc3RlaW4sIF MuPC9hdXRo b3I+PC9hdXRob3JzPjwvY29udHJpYnVOb3JzPjxhdXRoLWFkZHJlc3M+VW5pdmVyc 210eSBvZiBQ aXROc2J1cmdoIENhbmNlciBJbnN0aXR1dGUsIFBpdHRzYnVyZ2gsIFBlbm5zeWx2Y W5pYSAxNTIz MiwgVVNBLiByYW1hbmFOaGFucmtAbXN4LnVwbWMuZWR1PC9hdXRoLWFkZH J1c3M+PHRpdGxlcz48 dGl0bGU+TG93IG92ZXJ1eHByZXNzaW9uIG9mIEhFUiOyL251dSBpbiBhZHZhbmN1Z CBjb2xvcmVj dGFsIGNhbmNlciBsaW1pdHMgdGhllHVzZWZ1bG5lc3Mgb2YgdHJhc3R1enVtYWIg KEhlcmNlcHRp bikgYW5kIGlyaW5vdGVjYW4gYXMgdGhlcmFweS4gQSBwaGFzZSBJSSBOcmlhbD wvdGl0bGU+PHN1 Y29uZGFyeS1OaXRsZT5DYW5jZXIgSW52ZXNOPC9zZWNvbmRhcnktdGl0bGU+PG FsdC1OaXRsZT5D YW5jZXIgaW52ZXNOaWdhdGlvbjwvYWx0LXRpdGxlPjwvdGl0bGVzPjxwZXJpb2Rp Y2FsPjxmdWxs LXRpdGxlPkNhbmNlciBJbnZlc3Q8L2Z1bGwtdGl0bGU+PGFiYnItMT5DYW5jZXIga W52ZXNOaWdh dGlvbjwvYWJici0xPjwvcGVyaW9kaWNhbD48YWx0LXBlcmlvZGljYWw+PGZ1bGw tdG10bGU+Q2Fu Y2VyIEludmVzdDwvZnVsbC1OaXRsZT48YWJiciOxPkNhbmNlciBpbnZlc3RpZ2FOaW 9uPC9hYmJy LTE+PC9hbHQtcGVyaW9kaWNhbD48cGFnZXM+ODU4LTY1PC9wYWdlcz48dm9s dWllPjIyPC92b2xl bWU+PG51bWJlcj42PC9udWliZXI+PGtleXdvcmRzPjxrZX13b3JkPkFkdWxOPC9rZX1 3b3JkPjxr ZX13b3JkPkFnZWQ8L2tleXdvcmQ+PGtleXdvcmQ+QW50aWJvZGllcywgTW9ub2Nsb 25hbC9hZGlp bmlzdHJhdGlvbiAmYW1wOyBkb3NhZ2U8L2tleXdvcmQ+PGtleXdvcmQ+QW50aWJ vZGllcywgTW9u b2Nsb25hbCwgSHVtYW5pemVkPC9rZX13b3JkPjxrZX13b3JkPkFudGluZW9wbGFzdG ljIENvbWJp bmVkIENoZW1vdGhlcmFweSBQcm90b2NvbHMvcGhhcm1hY29raW5ldGljcy8qdGhlc mFwZXVOaWMg dXN1PC9rZX13b3JkPjxrZX13b3JkPkNhbXBOb3RoZWNpbi9hZGlpbmlzdHJhdGlvbiA mYW1wOyBk b3NhZ2UvKmFuYWxvZ3MgJmFtcDsgZGVyaXZhdG12ZXM8L2tleXdvcmQ+PGtleXd vcmQ+Q29sb3Jl Y3RhbCBOZW9wbGFzbXMvKmRydWcgdGhlcmFweS8qbWVOYWJvbGlzbS9wYXR ob2xvZ3k8L2tleXdv cmQ+PGtleXdvcmQ+RmVtYWx1PC9rZX13b3JkPjxrZX13b3JkPkdlbmUgRXhwcmVzc2 lvbiBSZWdl bGFOaW9uLCBOZW9wbGFzdGljPC9rZX13b3JkPjxrZX13b3JkPkhlbWFuczwva2V5d2 9yZD48a2V5 d29yZD5NYWx1PC9rZX13b3JkPjxrZX13b3JkPk1heGltdWOgVG9sZXJhdGVkIERvc2U 8L2tleXdv cmQ+PGtleXdvcmQ+TW1kZGx1IEFnZWQ8L2tleXdvcmQ+PGtleXdvcmQ+UmVjZXB Ob3IsIGVyYkIt Mi9nZW5ldGljcy8qbWVOYWJvbGlzbTwva2V5d29yZD48a2V5d29yZD5UcmVhdGllb nQgT3VOY29t ZTwva2V5d29yZD48L2tleXdvcmRzPjxkYXRlcz48eWVhcj4yMDAOPC95ZWFyPjwvZ GF0ZXM+PGlz Ym4+MDczNSO3OTA3IChQcmludCkmI3hEOzA3MzUtNzkwNyAoTGlua2luZyk8L21z Ym4+PGFjY2Vz c2lvbiludWO+MTU2NDEOODM8L2FjY2Vzc2lvbiludWO+PHVybHM+PHJlbGFOZW QtdXJscz48dXJs PmhOdHA6Ly93d3cubmNiaS5ubGOubmloLmdvdi9wdWJtZWQvMTU2NDE0ODM8L3 VybD48L3JlbGF0 ZWQtdXJscz48L3VybHM+PC9yZWNvcmQ+PC9DaXRIPjwvRW5kTm90ZT4A PEVuZE5vdGU+PENpdGU+PEF1dGhvcj5SYW1hbmFOaGFuPC9BdXRob3I+PFllYXI+MjAwNDwvWWVh cj48UmVj TnVtPjE4PC9SZWNOdW0+PERpc3BsYX1UZXhOPjxzdHlsZSBmYWN1PSJ zdXBlcnNjemlw dCI+MjA8L3NOeWx1PjwvRGlzcGxheVRleHQ+PHJlY29yZD48cmVjLW51bWJlcj4xO DwvcmVjLW51 bWJlcj48Zm9yZWlnbi1rZXlzPjxrZXkgYXBwPSJFTiIgZGItaWQ9IndkdmQyeDJ3NDJ kOXgzZXh4 YW54NWRwZXJkZWRwdHRhMnhmeCI+MTg8L2tleT48L2ZvcmVpZ24ta2V5cz48c mVmLXR5cGUgbmFt ZT0iSm91cm5hbCBBcnRpY2xlIj4xNzwvcmVmLXR5cGU+PGNvbnRyaWJ1dG9ycz48 YXV0aG9ycz48 YXV0aG9yPlJhbWFuYXRoYW4sIFIuIEsuPC9hdXRob3I+PGF1dGhvcj5Id2FuZywgSi 4gSi48L2F1 dGhvcj48YXV0aG9yPlphbWJvbmksIFcuIEMuPC9hdXRob3I+PGF1dGhvcj5TaW5pY3 JvcGUsIEYu IEEuPC9hdXRob3I+PGF1dGhvcj5TYWZyYW4sIEguPC9hdXRob3I+PGF1dGhvcj5Xb 25nLCBNLiBL LjwvYXV0aG9yPjxhdXRob3I+RWFybGUsIEOuPC9hdXRob3I+PGF1dGhvcj5CcnVmc 2t5LCBBLjwv YXV0aG9yPjxhdXRob3I+RXZhbnMsIFQuPC9hdXRob3I+PGF1dGhvcj5Ucm9ldHNja GVsLCBNLjwv YXV0aG9yPjxhdXRob3I+V2Fsa28sIEMuPC9hdXRob3I+PGF1dGhvcj5EYXksIFIuPC9 hdXRob3I+PGF1dGhvcj5DaGVuLCBILiBYLjwvYXV0aG9yPjxhdXRob3I+Rmlua2Vsc3RlaW4sIF MuPC9hdXRo b3I+PC9hdXRob3JzPjwvY29udHJpYnVOb3JzPjxhdXRoLWFkZHJlc3M+VW5pdmVyc 210eSBvZiBQ aXROc2J1cmdoIENhbmNlciBJbnN0aXR1dGUsIFBpdHRzYnVyZ2gsIFBlbm5zeWx2Y W5pYSAxNTIz MiwgVVNBLiByYW1hbmFOaGFucmtAbXN4LnVwbWMuZWR1PC9hdXRoLWFkZH J1c3M+PHRpdGxlcz48 dGl0bGU+TG93IG92ZXJ1eHByZXNzaW9uIG9mIEhFUiOyL251dSBpbiBhZHZhbmN1Z CBjb2xvcmVj dGFsIGNhbmNlciBsaW1pdHMgdGhlIHVzZWZ1bG5lc3Mgb2YgdHJhc3R1enVtYWIg KEhlcmNlcHRp bikgYW5kIGlyaW5vdGVjYW4gYXMgdGhlcmFweS4gQ SBwaGFzZSBJSSBOcmlhbD wvdGl0bGU+PHN1 Y29uZGFyeS1OaXRsZT5DYW5jZXIgSW52ZXNOPC9zZWNvbmRhcnktdGl0bGU+PG FsdC1OaXRsZT5D YW5jZXIgaW52ZXNOaWdhdGlvbjwvYWx0LXRpdGxlPjwvdGl0bGVzPjxwZXJpb2Rp Y2FsPjxmdWxs LXRpdGxlPkNhbmNlciBJbnZlc3Q8L2Z1bGwtdGl0bGU+PGFiYnItMT5DYW5jZXIga W52ZXNOaWdh dGlvbjwvYWJici0xPjwvcGVyaW9kaWNhbD48YWx0LXBlcmlvZGljYWw+PGZ1bGw tdG10bGU+Q2Fu Y2VyIEludmVzdDwvZnVsbC1OaXRsZT48YWJiciOxPkNhbmNlciBpbnZlc3RpZ2FOaW 9uPC9hYmJy LTE+PC9hbHQtcGVyaW9kaWNhbD48cGFnZXM+ODU4LTY1PC9wYWdlcz48dm9s dWllPjIyPC92b2xl bWU+PG51bWJlcj42PC9udWliZXI+PGtleXdvcmRzPjxrZX13b3JkPkFkdWxOPC9rZX1 3b3JkPjxr ZX13b3JkPkFnZWQ8L2tleXdvcmQ+PGtleXdvcmQ+QW50aWJvZGllcywgTW9ub2Nsb 25hbC9hZGlp bmlzdHJhdGlvbiAmYW1wOyBkb3NhZ2U8L2tleXdvcmQ+PGtleXdvcmQ+QW50aWJ vZGllcywgTW9u b2Nsb25hbCwgSHVtYW5pemVkPC9rZX13b3JkPjxrZX13b3JkPkFudGluZW9wbGFzdG ljIENvbWJp bmVkIENoZW1vdGhlcmFweSBQcm90b2NvbHMvcGhhcm1hY29raW5ldGljcy8qdGhlc mFwZXVOaWMg dXN1PC9rZX13b3JkPjxrZX13b3JkPkNhbXBOb3RoZWNpbi9hZGlpbmlzdHJhdGlvbiA mYW1wOyBk b3NhZ2UvKmFuYWxvZ3MgJmFtcDsgZGVyaXZhdG12ZXM8L2tleXdvcmQ+PGtleXd vcmQ+Q29sb3Jl Y3RhbCBOZW9wbGFzbXMvKmRydWcgdGhlcmFweS8qbWVOYWJvbGlzbS9wYXR ob2xvZ3k8L2tleXdv cmQ+PGtleXdvcmQ+RmVtYWxlPC9rZX13b3JkPjxrZX13b3JkPkdlbmUgRXhwcmVzc2 lvbiBSZWdl bGFOaW9uLCBOZW9wbGFzdGljPC9rZX13b3JkPjxrZX13b3JkPkhlbWFuczwva2V5d2 9yZD48a2V5 d29yZD5NYWxlPC9rZX13b3JkPjxrZX13b3JkPk1heGltdWOgVG9sZXJhdGVkIERvc2U 8L2tleXdv cmQ+PGtleXdvcmQ+TWlkZGxlIEFnZWQ8L2tleXdvcmQ+PGtleXdvcmQ+UmVjZXB Ob3IsIGVyYkIt Mi9nZW5ldGljcy8qbWVOYWJvbGlzbTwva2V5d29yZD48a2V5d29yZD5UcmVhdGllb nQgT3VOY29t ZTwva2V5d29yZD48L2tleXdvcmRzPjxkYXRlcz48eWVhcj4yMDAOPC95ZWFyPjwvZ GF0ZXM+PGlz Ym4+MDzNSO3OTA3IChQcmludCkmI3hEOzA3MzUtNzkwNyAoTGlua2luZyk8L21z Ym4+PGFjY2Vz c2lvbiludWO+MTU2NDEOODM8L2FjY2Vzc2lvbiludWO+PHVybHM+PHJlbGFOZW QtdXJscz48dXJs PmhOdHA6Ly93d3cubmNiaS5ubGOubmloLmdvdi9wdWJtZWQvMTU2NDE0ODM8L3 VybD48L3JlbGF0 ZWQtdXJscz48L3VybHM+PC9yZWNvcmQ+PC9DaXRlPjwvRW5kTm90ZT4A 20. Small molecule inhibitors have been reported that target proteins or the pathways encoded by the altered genes identified, including PIK3CA, BRAF, AKT1/AKT2, and MDM2, but these not been evaluated in pancreatic cancer.

This study highlights information that may be obtained through the integration of large-scale genomic and clinical analyses in pancreatic cancer. Although careful measures were taken to increase sensitivity of detecting genetic changes in the tumors and in the circulation of these patients, some alterations may not have been detected due to low tumor purity, limited plasma amounts, and low mutant allele frequency. Despite these limitations, these data add to our growing understanding of pancreatic cancer.

### REFERENCES

The disclosure of each reference cited is expressly incorporated herein.
1. Siegel, R. L., Miller, K. D. & Jemal, A. Cancer statistics, 2015. CA Cancer J Clin 65, 5-29 (2015).
2. Biankin, A. V. et al. Pancreatic cancer genomes reveal aberrations in axon guidance pathway genes. Nature 491, 399-405 (2012).
3. Jones, S. et al. Core signaling pathways in human pancreatic cancers revealed by global genomic analyses. Science 321, 1801-6 (2008).
4. Paulson, A. S., Tran Cao, H. S., Tempero, M. A. & Lowy, A. M. Therapeutic advances in pancreatic cancer. Gastroenterology 144, 1316-26 (2013).
5. Siegel, R., Naishadham, D. & Jemal, A. Cancer statistics, 2013. CA Cancer J Clin 63, 11-30 (2013).
6. Jones, S. et al. Somatic mutations in the chromatin remodeling gene ARID1A occur in several tumor types. Hum Mutat 33, 100-3 (2012).
7. Goyama, S. & Mulloy, J. C. NF-kappaB: A Coordinator for Epigenetic Regulation by MLL. Cancer Cell 24, 401-2 (2013).
8. Popovic, R. & Licht, J. D. Emerging epigenetic targets and therapies in cancer medicine. Cancer Discov 2, 405-13 (2012).
9. Jiao, Y. et al. DAXX/ATRX, MEN1, and mTOR pathway genes are frequently altered in pancreatic neuroendocrine tumors. Science 331, 1199-203 (2011).
10. Sausen, M. et al. Integrated genomic analyses identify ARID1A and ARID1B alterations in the childhood cancer neuroblastoma. Nat Genet 45, 12-7 (2013).
11. Bettegowda, C. et al. Detection of circulating tumor DNA in early- and late-stage human malignancies. Sci Transl Med 6, 224ra24 (2014).
12. Newman, A. M. et al. An ultrasensitive method for quantitating circulating tumor DNA with broad patient coverage. Nat Med 20, 548-54 (2014).
13. Iacobuzio-Donahue, C. A., Velculescu, V. E., Wolfgang, C. L. & Hruban, R. H. Genetic basis of pancreas cancer development and progression: insights from whole-exome and whole-genome sequencing. Clin Cancer Res 18, 4257-65 (2012).
14. Forbes, S. A. et al. COSMIC: mining complete cancer genomes in the Catalogue of Somatic Mutations in Cancer. Nucleic Acids Res 39, D945-50 (2011).
15. Fogelman, D. R. et al. Evidence for the efficacy of Iniparib, a PARP-1 inhibitor, in BRCA2-associated pancreatic cancer. Anticancer Res 31, 1417-20 (2011).
16. Fong, P. C. et al. Inhibition of poly(ADP-ribose) polymerase in tumors from BRCA mutation carriers. N Engl J Med 361, 123-34 (2009).
17. van der Heijden, M. S. et al. In vivo therapeutic responses contingent on Fanconi anemia/BRCA2 status of the tumor. Clin Cancer Res 11, 7508-15 (2005).
18. Villarroel, M. C. et al. Personalizing cancer treatment in the age of global genomic analyses: PALB2 gene mutations and the response to DNA damaging agents in pancreatic cancer. Mol Cancer Ther 10, 3-8 (2011).
19. Sorscher, S. M. Marked response to single agent trastuzumab in a patient with metastatic HER-2 gene amplified rectal cancer. Cancer Invest 29, 456-9 (2011).
20. Ramanathan, R. K. et al. Low overexpression of HER-2/neu in advanced colorectal cancer limits the usefulness of trastuzumab (Herceptin) and irinotecan as therapy. A phase II trial. Cancer Invest 22, 858-65 (2004).
20. Jiao, Y. et al. DAXX/ATRX, MEN1, and mTOR pathway genes are frequently altered in pancreatic neuroendocrine tumors. Science 331, 1199-203 (2011).
21. Jones, S. et al. Core signaling pathways in human pancreatic cancers revealed by global genomic analyses. Science 321, 1801-6 (2008).
22. Sausen, M. et al. Integrated genomic analyses identify ARID1A and ARID1B alterations in the childhood cancer neuroblastoma. Nat Genet 45, 12-7 (2013).
23. Cooper, G. M. et al. Distribution and intensity of constraint in mammalian genomic sequence. Genome Res 15, 901-13 (2005).

We claim:

1. A method for characterizing a pancreatic adenocarcinoma, comprising:
testing a sample of nucleic acids from a pancreatic adenocarcinoma of a human for a somatic mutation in a chromatin regulating gene selected from the group consisting of MLL, MLL2, MLL3, and ARID1A.

2. The method of claim 1 wherein the somatic mutation is detected by testing nucleic acids from a tissue of the human which is not pancreatic adenocarcinoma.

3. The method of claim 1 wherein the sample is from a tumor.

4. The method of claim 1 wherein the sample is from plasma.

5. The method of claim 1 wherein the somatic mutation is a missense mutation or a truncating mutation.

6. The method of claim 1 wherein the sample is tested for a mutation in ARID1A selected from the group consisting of:

| | | | |
|---|---|---|---|
| chr1_26928798- | 403Q > X | Substitution | Nonsense; |
| 26928798_C_T | | | |
| chr1_26979465- | NA | Insertion | Frameshift; |
| 26979465_C | | | |
| chr1_26978312- | 1779E > G | Substitution | Nonsynonymous |
| 26978312_A_G | | | coding; |
| chr1_26978589- | 1871H > Q | Substitution | Nonsynonymous |
| 26978589_C_G | | | coding; |
| chr1_26895595- | 38E > EA | Insertion | In-frame |
| 26895595_GGC | | | insertion; |
| chr1_26960135- | 708Q > X | Substitution | Nonsense; |
| 26960135_C_T | | | |
| chr1_26973560- | 1419A > T | Substitution | Nonsynonymous |
| 26973560_G_A | | | coding; |
| chr1_26974706- | 1682A > E | Substitution | Nonsynonymous |
| 26974706_C_A | | | coding; |
| chr1_26972534- | 1276R > X | Substitution | Nonsense; |
| 26972534_C_T | | | |
| chr1_26978518- | NA | Deletion | Frameshift; |
| 26978518_G_ | | | and |
| chr1_26979254- | NA | Insertion | Frameshift; |
| 26979254_TT | | | |

7. The method of claim 1 wherein the sample is tested for a mutation in an MLL (chromosome 11), MLL2 (chromosome 12), or MLL 3 (chromosome 7) gene selected from the group consisting of:

| | | | |
|---|---|---|---|
| chr7_151490485- | 3704V > L | Substitution | Nonsynonymous |
| 151490485_C_G | | | coding; |
| chr11_117854039- | 1161C > S | Substitution | Nonsynonymous |
| 117854039_G_C | | | coding; |
| chr11_117847769- | 229P > T | Substitution | Nonsynonymous |
| 117847769_C_A | | | coding; |
| chr12_47718147- | 3087R > W | Substitution | Nonsynonymous |
| 47718147_G_A | | | coding; |
| chr7_151515732- | NA | Substitution | Splice |
| 151515732_C_T | | | site donor; |
| chr12_47729834- | NA | Insertion | Frameshift; |
| 47729834_A | | | |
| chr7_151576224- | 743P > L | Substitution | Nonsynonymous |
| 151576224_G_A | | | coding; |
| chr7_151476195- | 4584R > W | Substitution | Nonsynonymous |
| 151476195_G_A | | | coding; |
| chr7_l51686622- | NA | Insertion | Frameshift; |
| 151686622_TC | | | |
| chr12_47711653- | NA | Insertion | Frameshift; |
| 47711653_T | | | |
| chr12_47731362- | NA | Deletion | Frameshift; |
| 47731362_G_ | | | |
| chr7_151601824- | NA | Insertion | Frameshift; |
| 151601824_T | | | |
| chr12_47722327- | 1974T > M | Substitution | Nonsynonymous |
| 47722327_G_A | | | coding; and |
| chr7_151510210- | 1890R > X | Substitution | Nonsense. |
| 151510210_G_A | | | |

8. A method for testing a human comprising:
testing plasma of a human collected at the time of diagnosis of a pancreatic adenocarcinoma for the presence of cell-free tumor DNA, wherein the cell-free tumor DNA contains a somatic mutation present in the pancreatic adenocarcinoma but not present in tissues that are not pancreatic adenocarcinoma.

9. A method for testing a human comprising:
testing plasma of a human collected after surgical resection of a pancreatic adenocarcinoma for the presence of cell-free tumor DNA, wherein the cell-free tumor DNA contains a somatic mutation present in the pancreatic adenocarcinoma but not present in tissues that are not pancreatic adenocarcinoma.

10. The method of claim 8 wherein the somatic mutation is a missense mutation or a truncating mutation.

11. The method of claim 9 wherein the somatic mutation is a missense mutation or a truncating mutation.

12. A probe or primer specific for a mutant chromatin regulating MLL (chromosome 11), MLL2 (chromosome 12), MLL 3 (chromosome 7) or ARID1A gene, said probe or primer comprising a mutation selected from the group consisting of: ARID1A mutations:

| | | | |
|---|---|---|---|
| chr1_26928798- | 403Q > X | Substitution | Nonsense; |
| 26928798_C_T | | | |
| chr1_26979465- | NA | Insertion | Frameshift; |
| 26979465_C | | | |
| chr1_26978312- | 1779E > G | Substitution | Nonsynonymous |
| 26978312_A_G | | | coding; |
| chr1_26978589- | 1871H > Q | Substitution | Nonsynonymous |
| 26978589_C_G | | | coding; |
| chr1_26895595- | 38E > EA | Insertion | In-frame |
| 26895595_GGC | | | insertion; |
| chr1_26960135- | 708Q > X | Substitution | Nonsense; |
| 26960135_C_T | | | |
| chr1_26973560- | 1419A > T | Substitution | Nonsynonymous |
| 26973560_G_A | | | coding; |
| chr1_26974706- | 1682A > E | Substitution | Nonsynonymous |
| 26974706_C_A | | | coding; |
| chr1_26972534- | 1276R > X | Substitution | Nonsense; |
| 26972534_C_T | | | |
| chr1_26978518- | NA | Deletion | Frameshift; |
| 26978518_G_ | | | and |
| chr1_26979254- | NA | Insertion | Frameshift; |
| 26979254_TT | | | |

and MLL (chromosome 11), MLL2 (chromosome 12), or MLL 3 (chromosome 7) mutations:

| | | | |
|---|---|---|---|
| chr7_151490485- | 3704V > L | Substitution | Nonsynonymous |
| 151490485_C_G | | | coding; |
| chr11_117854039- | 1161C > S | Substitution | Nonsynonymous |
| 117854039_G_C | | | coding; |
| chr11_117847769- | 229P > T | Substitution | Nonsynonymous |
| 117847769_C_A | | | coding; |
| chr12_47718147- | 3087R > W | Substitution | Nonsynonymous |
| 47718147_G_A | | | coding; |
| chr7_151515732- | NA | Substitution | Splice |
| 151515732_C_T | | | site donor; |
| chr12_47729834- | NA | Insertion | Frameshift; |
| 47729834_A | | | |
| chr7_151576224- | 743P > L | Substitution | Nonsynonymous |
| 151576224_G_A | | | coding; |
| chr7_151476195- | 4584R > W | Substitution | Nonsynonymous |
| 151476195_G_A | | | coding; |
| chr7_l51686622- | NA | Insertion | Frameshift; |
| 151686622_TC | | | |
| chr12_47711653- | NA | Insertion | Frameshift; |
| 47711653_T | | | |
| chr12_47731362- | NA | Deletion | Frameshift; |
| 47731362_G_ | | | |
| chr7_151601824- | NA | Insertion | Frameshift; |
| 151601824_T | | | |
| chr12_47722327- | 1974T > M | Substitution | Nonsynonymous |
| 47722327_G_A | | | coding; and |
| chr7_151510210- | 1890R > X | Substitution | Nonsense. |
| 151510210_G_A | | | |

13. The probe or primer of claim 12 which is labeled with a radionuclide.

14. The probe or primer of claim 12 which is labeled with a fluorescent label.

15. The probe or primer of claim 12 which is labeled with a chromophore.

16. The probe or primer of claim 12 wherein the probe or primer is complementary to the sense strand.

17. The method of claim 1 wherein upon identifying the somatic mutation, providing to the human a prediction of increased overall survival and increased progression free survival, relative to humans without the somatic mutation.

18. The method of claim 1 wherein upon identifying the somatic mutation, decreasing the intensity of therapy for the human.

19. The method of claim 1 wherein upon identifying no somatic mutation in the chromatin regulating genes providing to the human a prediction of decreased overall survival and decreased progression free survival, relative to humans with the somatic mutation.

20. The method of claim 1 wherein upon identifying no somatic mutation in the chromatin regulating genes, increasing the intensity of therapy for the human.

21. The method of claim 1 wherein the sample is from a body fluid that drains from pancreata.

22. The method of claim 18 wherein the sample is obtained and tested prior to any surgical resection, and surgical resection is averted.

23. The method of claim 8 wherein upon detection of cell-free tumor DNA, providing to the human a prediction of increased risk of disease recurrence after surgical resection, relative to humans with pancreatic adenocarcinomas that do not have detectable cell-free tumor DNA.

24. The method of claim 9 wherein upon detection of cell-free tumor DNA, providing to the human a prediction of increased risk of disease recurrence after surgical resection, relative to humans with pancreatic adenocarcinomas that do not have detectable cell-free tumor DNA.

25. The method of claim 8 wherein the cell-free tumor DNA is a mutant gene selected from the group consisting of KRAS, BRAF, and PIK3CA.

26. The method of claim 9 further comprising the step of monitoring for disease recurrence using computed tomography.

27. The method of claim 8 wherein when cell-free tumor DNA is present, increasing intensity of therapy for the human.

28. The method of claim 9 wherein when cell-free tumor DNA is present, increasing intensity of therapy for the human.

29. The method of claim 8 wherein when cell-free tumor DNA is not detected, decreasing intensity of therapy for the human.

30. The method of claim 9 wherein when cell-free tumor DNA is not detected, decreasing intensity of therapy for the human.

31. The method of claim 9 further comprising the step of testing DNA from the pancreatic adenocarcinoma and identifying a somatic mutation prior to testing the plasma.

32. The method of claim 1 wherein a somatic mutation is identified.

33. The method of claim 8 wherein a somatic mutation is identified.

34. The method of claim 9 wherein a somatic mutation is identified.
